(19) United States
(12) Patent Application Publication
(10) Pub. No.: US 2024/0327323 A1
(43) Pub. Date: Oct. 3, 2024

(54) Methods of Treating of Halogen- Containing Waste Plastic to Produce Halogenated Products

(71) Applicant: REGENTS OF THE UNIVERSITY MICHIGAN, Ann Arbor, MI (US)

(72) Inventors: Anne J. McNeil, Ann Arbor, MI (US); Danielle Fagnani, Ann Arbor, MI (US); Dukhan Kim, Ann Arbor, MI (US); Rahul Jha, Ann Arbor, MI (US)

(21) Appl. No.: 18/584,756

(22) Filed: Feb. 22, 2024

Related U.S. Application Data
(60) Provisional application No. 63/486,955, filed on Feb. 24, 2023.

(51) Int. Cl. C07C 41/22; C25B 3/11; C25B 3/25; C25B 3/27

(52) U.S. Cl. CPC C07C 41/22; C25B 3/11; C25B 3/25; C25B 3/27

(57) ABSTRACT

A method for halogenation of an organic compound with a halogen from a waste source comprising a halogen containing polymer includes admixing waste source with the organic compound, a solvent, and an electrolyte, wherein the solvent swells and/or dissolves the halogen containing polymer in the waste source; and exposing the admixture to a negative voltage in an electrochemical cell comprising a cathode and an oppositely disposed anode. The halogen containing polymer is dehalogenated by the cathode to generate a halogen anion. The halogen anode is formed into a reactive halogen species by the anode. The halogen species reacts with the organic compound to halogenate the organic compound

### CROSS-REFERENCE TO RELATED APPLICATION

The benefit of priority to U.S. Provisional Patent Application No. 63/486,955 filed Feb. 24, 2023, is hereby claimed and the disclosure is incorporated herein by reference in its entirety.

### FIELD

The disclosure generally relates to method of de-halogenating waste polymers containing halogens to generate reactive halogen species for halogenation of organic compounds.

### BACKGROUND

Plastics recycling has until now been predominately focused on mechanical recycling, which downgrades material quality and therefore limits its utility. Recycling rate (<10%) are unlikely to improve with mechanical recycling alone. Closed-loop chemical recycling, in which polymers are first depolymerized and then repolymerized to generate pristine material, has only been realized for hydrolyzable polymers such as poly(ethylene terephthalate (PET). Polymers with all-carbon backbones are not thermodynamically amenable to such systems. Such polymer can more practically be recycled in an open-loop process, wherein polymers are broken down into smaller fragments and/or transformed into different materials that serve as feedstocks in other chemical processes. The leading open-loop process is high-temperature anaerobic pyrolysis. It is only useful for hydrocarbon polymers such as polyethylene, polypropylene, and polystyrene.

PVC has the highest production rate, but has the lowest recycling rate in most countries, with 0.6% in the U.S. PVC poses known health risk in the environment. PVC has been detected in wastewater sludge and in aquatic life commonly consumed by humans. It is ranked one of the most hazardous polymers, in part due to the plasticizing additive such as phthalates, which are carcinogenic and mutagenic, as well as due to the corrosive chlorine-containing by-products that are formed upon degradation. These additives and by-products make it difficult to recycle PVC via mechanical approaches, requiring separation of the PVC from other plastics to avoid contamination. Because complete separation is challenging, common PVC plasticizers such as, di-(2-ethylhexyl) phthalate (DEHP) and other phthalates such as dioctyl terephthalate (DOTP) are often found in other recycled plastics.

Even without additives, PVC remains difficult to recycle by melt-processing or pyrolysis because hydrochloric acid (HCl) and other volatiles are rapidly eliminated upon heat treatment. HCl poses safety hazards and corrodes equipment.

Conventional processes for controlled PVC dechlorination rely on either thermal activation or stoichiometric strong bases. For example, hydrothermal treatment degrades DEHP and dechlorinates PVC at temperature of 250° C. in pressurized systems with catalysts or other additives. While these treatments are useful for recovering the hydrocarbon content of PVC, the chlorine content (57% by mass) is waste.

### SUMMARY

HCl is a useful reagent in many chemical reactions. Methods of the disclosure advantageously allow for the use of waste PVC as a solid reagent that release HCl on demand. In situ generated HCl (or Cl₂) could find a range of synthetic applications. For example, more than 85% of pharmaceuticals employ chlorine chemistry during production. In 2020, over 9,000 kt of chlorine was manufactured in Europe with 33% used in PVC and 54% used in other organochlorine applications. The methods of the disclosure advantageously utilize the fraction of chlorine stored in PVC for later use in synthetic reactions. Additionally, intermediate polymer compositions formed in the processes of the disclosure, for example, through partial dechlorination, can be useful industrial products, as well.

In accordance with the disclosure, a method for halogenation of an organic compound with a reactive halogen species generated from a waste source comprising a halogen-containing polymer, can include admixing the waste source with the organic compound, a solvent, and an electrolyte, wherein the solvent swells and/or dissolves the halogen-containing polymer in the waste source; exposing the admixture to a negative voltage in an electrochemical cell comprising a cathode and an oppositely disposed anode. As a result, the halogen containing polymer is dehalogenated by the cathode to generate a halogen anion. The halogen anion is formed into a reactive halogen species by the anode. The halogen species reacts with the organic compound to halogenate the organic compound.

In accordance with the disclosure, a method for halogenation of an organic compound with a reactive halogen species from a waste source comprising a halogen-containing polymer, can include admixing the waste source with, a solvent, and an electrolyte, wherein the solvent swells and/or dissolves the halogen-containing polymer in the waste source; exposing the admixture to a negative voltage in an electrochemical cell comprising a cathode by which the halogen containing polymer is dehalogenated and halogen anions are generated, and an oppositely disposed anode by which the halogen anions are transformed into a reactive halogen species; recovering the reactive halogen species from the electrochemical cell; and admixing the reactive halogen species with the organic compound, wherein the reactive halogen species reacts with the organic compound to halogenate the organic compound.

### BRIEF DESCRIPTION OF THE DRAWINGS

FIG. 1A shows a general scheme for reductive dechlorination of PVC (cathode) and oxidative chlorination of an arene (anode).

FIG. 1B shows a scheme of the model reaction and conditions evaluated. Ethoxybenzyene (1) is chlorinated using PBV and DEHP.

FIG. 1C is a summary of the results showing that the chlorination reaction proceeded to higher yields with the lower molar mass PVC and that reactivity can be turned on/off with the plasticizer present/absent under constant-voltage conditions. The reported yields are an average of two runs ±s.d., and were measured by GC-MS, n.r., no reaction.

FIGS. 2A to 2C show representative cyclic voltammetry data: cyclic voltammograms of the first reduction of DEHP with and without PVC47k (30 equiv.) collected at 100 mV s⁻¹ (A), redox reversibility as a function of excess factor measured in triplicate (see Table 17 for data with s.d.) at several scan rates (25, 50, 100, 250 and 500 mV s⁻¹) (B), and a scheme depicting the standard electrochemical pathway for the single reduction of DEHP (left) and the alternative pathway when PVC is added (right), which is suggested by the cyclic voltammetry data (C).

FIG. 2D to 2F are schematics of the bulk electrolysis set-up and reactions occurring at each electrode (D, voltage readout from −5 mA constant-current electrolysis (E) and current readout from −2.5 V constant-voltage electrolysis (F). These data show that PVC can be reduced at smaller voltages with DEHP present, indicating that DEHP functions as a redox mediator.

FIG. 3A for ¹H NMR spectra (500 MHz, THF-d⁸). PVC₄₆ₖ and dPVC_{DMP}. A large excess of phthalate was used to increase the NMR signal intensity of substituted products in the resultant polymer (dPVC_{DMP}). The peaks associated with the solvent (THF), water and silicone grease are marked by asterisks.

FIG. 3B is the chemical structures of probable repeat units in dPVC_{DMP}—including a complex mixture of substituents as well as unreacted PVC units—compared with unreacted _{PVC47k}.

FIG. 4 is a schematic of a redox-mediated paired-electrolysis mechanism. The following steps are numbered on the schematic: (1) reduction of plasticizer; (2) electron transfer to polymer; (3) dechlorination of polymer; (4) chloride oxidation; and (5) oxidative chlorination of the arene. Major products observed from the reaction are highlighted in the boxes.

FIG. 5A shows images of Poly(vinyl chloride) tubing used to simulate PVC waste.

FIG. 5B is a chart showing yields of galvanostatic reaction using arene 1 as the chlorination substrate.

FIG. 5C shows images of plastic items used to simulate a mixed-plastics-waste stream. The mixed-plastics reaction contained by mass: 10% PET, 13% HDPE, 15% LDPE, 9% LLDPE, 13% polypropylene (PP), 9% polystyrene (not shown) and 31% PVC. The higher yield for PVC_{tubing} compared with PVC_{solid} demonstrates that plasticized PVC waste can be used directly without adding plasticizer.

FIG. 6 is a schematic of a divided cell reaction set-up for the paired electrolysis reaction and substrates utilized, with yields reported.

FIG. 7 is a graph showing normalized SEC traces of PVC samples.

FIG. 8 is stacked ¹H NMR spectra of DEHP standard (Sigma Aldrich) and liquid extracted from Tygon tubing sample. DEHP standard ¹H NMR (500 MHz, CDCl3) δ 7.74-7.67 (m, 2H), 7.55-7.49 (m, 2H), 4.28-4.16 (m, 4H), 1.74-1.61 (m, 2H), 1.49-1.24 (m, 16H), 0.97-0.84 (m, 12H). ¹H NMR (500 MHz, CDCl3) δ 7.74-7.68 (m, 2H), 7.56-7.49 (m, 2H), 4.27-4.16 (m, 4H), 1.76-1.63 (m, 2H), 1.51-1.24 (m, 16H), 0.96-0.85 (m, 12H).

FIG. 9 Stacked ¹³C NMR spectra of DEHP standard (Sigma Aldrich) and liquid extracted from Tygon tubing sample. DEHP standard ¹³C NMR (126 MHz, CDCl3) δ 167.85, 132.58, 130.98, 128.91, 68.26, 38.86, 30.49, 29.05, 23.88, 23.11, 14.17, 11.08. Tygon tubing extract ¹³C NMR (126 MHz, CDCl3) δ 167.88, 132.60, 131.01, 128.93, 68.28, 38.87, 30.50, 29.06, 23.88, 23.12, 14.18, 11.09.

FIG. 10 Stacked GC-MS chromatograms of DEHP standard (Sigma Aldrich) and liquid extracted from Tygon tubing sample.

FIGS. 11A and 11B are graphs showing monitoring conversion of 1 (●), consumption of DEHP (▪), and yield of 2 (▴) over time under 3 mA (A) and 7 mA (B) constant current. Reactions were performed on a 3 mL scale under N₂.

FIG. 12 is a graph showing monitoring conversion of 1 (●), consumption of DEHP (▪), and yield of 2 (▴) over time under −1.3 V constant voltage. Reaction was performed on a 3 mL scale under N₂.

FIGS. 13A and 13B are graphs showing monitoring conversion of (●), consumption of DEHP (▪), and yield of 2 (▴) over time under air (A) and a N₂ atmosphere (B). Reactions were performed on an 8 mL scale.

FIG. 14 is overlaid GC-MS chromatograms of chemical standards used in the examples.

FIG. 15 is representative GC-MS collected before and after the electrochemical reaction performed according to General Procedure C in the examples, using PVC₄ₖ and 1 equiv. of DEHP under 10 mA constant current. GC-MS peak at 3.53 min matches with 2-ethylhexanol.

FIG. 16 is a representative GC-MS collected before and after the electrochemical reaction performed according to General Procedure D, using PVC₄₇ₖ and 0 equiv. of DEHP under 10 mA constant current. No material eluted after 21 min.

FIG. 17 is a representative GC-MS collected before and after the electrochemical reaction performed according to General Procedure C, using PVC₄₇ₖ and 1 equiv. of DEHP under −1.3 V constant voltage. No material eluted after 21 min.

FIG. 18 is a graph showing the CVs of blank solution, 10 mM PVC₃₅ₖ calculated per repeat unit, and 10 mM DEHP. CVs were collected in 0.1 M NBu₄BF₄ in DMF using a 100 mV/s scan rate.

FIGS. 19A and 19B. CVs of DEHP at 10 mM (A) and 1 mM (B) and DEHP mixed with 30 equiv. PVC₃₅ₖ, PVC₁₀₀ₖ, or PS. CVs were collected in 0.1 M NBu₄BF₄ in DMF using 100 mV/s scan rate.

FIGS. 20A to 20G are cyclic voltammograms of 1 mM DEHP in the presence of (A) 0 equiv. (B) 1 equiv. (C) 10 equiv. (D) 30 equiv. of PVC35k with scan rates of 25, 50, 100, 250, 500 mV/s (darkest blue=25 and lightest blue=500 mV/s) (E) oxidation current (F) reduction current (G) oxidation/reduction. The averages and standard deviations (represented by the error bars) can be found in Table 16.

FIGS. 21A to 21G are cyclic voltammograms of 1 mM DEHP in the presence of (A) 0 equiv. (B) 1 equiv. (C) 10 equiv. (D) 30 equiv. of PVC47k with scan rates of 25, 50, 100, 250, 500 mV/s (darkest blue=25 and lightest blue=500 mV/s) (E) oxidation current (F) reduction current (G) oxidation/reduction. The averages and standard deviations (represented by the error bars) can be found in Table 16.

FIG. 22A to 22G are cyclic voltammograms of 1 mM DEHP in the presence of (A) 0 equiv. (B) 1 equiv. (C) 10 equiv. (D) 30 equiv. of PVC100k with scan rates of 25, 50, 100, 250, 500 mV/s (darkest blue=25 and lightest blue=500 mV/s (E) oxidation current (F) reduction current (G) oxidation/reduction. The averages and standard deviations (represented by the error bars) can be found in Table 16.

FIG. 22H is Table 16 containing raw data extracted from CV measurements, measured in triplicate using PVC₃₅ₖ as the PVC source.

FIG. 22I is Table 17 containing raw data extracted from CV measurements, measured in triplicate using PVC₄₇ₖ as the PVC source.

FIG. 22J is Table 18 containing raw data extracted from CV measurements, measured in triplicate using PVC₁₀₀ₖ as the PVC source.

FIGS. 23A to 23C are GC-MS traces of each half-reaction after constant current bulk electrolysis.

FIG. 24 is an image PVC_{tubing} and PVC_{tubing} cut up into smaller pieces.

FIG. 25 is a photograph of a reaction mixture containing PVC_{tubing} and pieces of mixed plastic items.

FIG. 26 is a photograph of dPVC recovered from reactions performed using PVC₄₇ₖ with 1 equiv. of DEHP (dPVC_{DEHP}, left) and with no DEHP (dPVCₐₗₒₙₑ, right).

FIG. 27 is a graph of normalized SEC traces of PVC and dPVC samples from standard conditions (RI detection). All samples were run in the same batch.

FIG. 28 is a graph showing TGA mass loss profiles collected from PVC47k (black) and dPVC recovered from reactions performed using PVC₄₇ₖ with 1 equiv. of DEHP (red) and with no DEHP (blue). The % mass remaining after the first stage is indicated on the plot. The first derivative was used to identify the completion of the first mass loss stage, designated when the first derivative reached zero.

FIG. 29 is a graph showing stacked ¹H NMR spectra (500 MHz, THF-d8) of dPVC recovered from reactions performed with DEHP and without DEHP and PVC47k.

FIG. 30A is a full IR spectra of PVC₄ₖ, dPVC recovered from reactions performed with DEHP and without DEHP,

FIG. 30B to 30E are the same IR spectra of FIG. 30A zoomed into smaller wavenumber regions.

FIG. 31 is DSC thermograms of PVC_{47K}, dPVC recovered from reactions performed with DEHP and without DEHP. The second heating/cooling cycle is shown.

FIG. 32 is a graph showing normalized SEC traces of PVC and dPVC sample from extended conditions (RI detection). All samples were run in the same batch. Molar mass data for PVC47k: Mn=57 kg/mol, Mw=123 kg/mol, ÐM=2.14; dPVCDMP: Mn=14 kg/mol, Mw=35 kg/mol, ÐM=2.14. Note, these samples were run several months apart those listed in Table 1, after repairs were made to the instrument.

FIG. 33 are images of pH strips color scale and pH strips immediately after being wetted with the reaction mixture.

FIG. 34 is a schematic of proposed reductive dichlorination mechanism and macroradical reaction pathways.

FIG. 35 is a schematic of a proposed oxidative arene chlorination mechanism.

FIG. 36 includes images showing a process flow for performing the method in an undivided cell.

FIG. 37 includes images showing a process flow for performing the method in a divided cell.

FIG. 38 is a process flow diagram of a method of the disclosure and life cycle assessment.

FIGS. 39 to 57 are ¹H and ¹³C NMR spectra of the synthesized compounds as identified in the examples.

FIG. 58A shows the structures of p-Tolunitrile, trans-stilbene, and 2,2′-Bipyrdine.

FIG. 58B are cyclic voltammograms of p-Tolunitrile, trans-stilbene, and 2,2′-Bipyridine (10 mM each) with and without PVC₂₂ₖ (30 mM) at 0.1 M TBABF₄/DMF at 100 mV/s.

FIG. 58C is a graph showing constant-current bulk electrolysis of p-Tolunitrile, trans-stilbene, and 2,2′-Bipyridine (50 mM each) with PVC₂₂ₖ (400 mM) in 0.3 M TBABF₄/DMF at −5 mA.

FIG. 59A is a reaction scheme of phenetole chlorination using PVC in a mediator-free process in an undivided cell and associated table showing % Cl-phenetole as a function of time.

FIG. 59B is an image showing electrode fouling during electrolysis in an undivided cell processing the reaction scheme of FIG. 59A.

FIG. 59C is a reaction scheme for the divided cell reaction and associated graph showing the yield of % Cl-phenetole with lower PVC loading.

FIG. 60 is a graph showing a reaction scheme in a divided cell and associated graph showing the effect of water on a mediator-free process in accordance with the disclosure.

FIG. 61A show images of the two types of dPVC obtained after electrolysis in a mediator free process—dPVC_{film}, and insoluble polymer deposited on cathode and dPVC_{precip}, a precipitate that precipitated out during workup.

FIG. 61B is a chart showing the elemental analysis data of the two dPVC types, confirming >90% dechlorination of dPVC_{film} and 60% dechlorination of dPVC_{precip},

FIG. 61C is a graph showing the thermogravimetric analysis of the two dPVC types, showing >30% mass residue of dPVC_{film} and dPVC_{precip} at 800° C.

### DETAILED DESCRIPTION

Methods of the disclosure can provide a paired electrolysis process for halogenation of an organic compound with halogen from a waste source comprising a halogen-containing polymer. The method can be done in a single-pot reaction in which the organic compound to be halogenated with the recovered halogen is present with the waste source during the reaction or in a separated process in which the halogenation reaction is performed separate from the dehalogenation of the waste source.

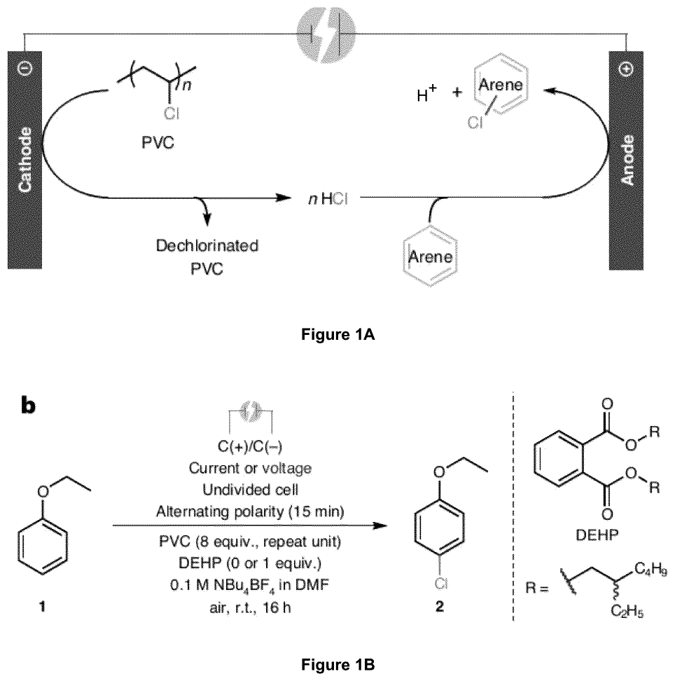

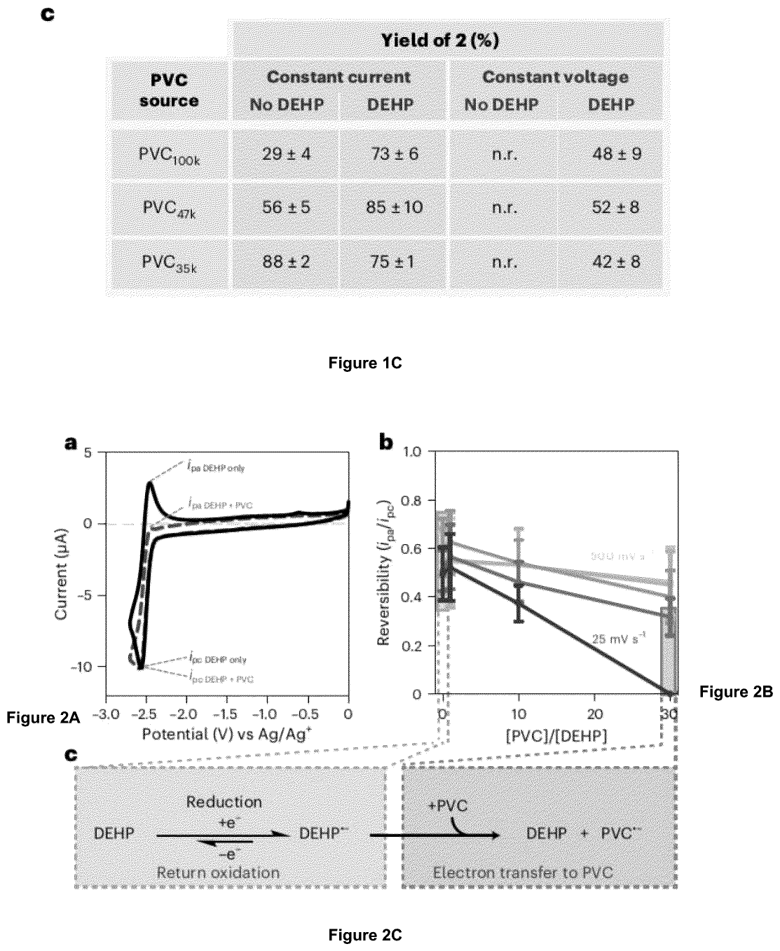

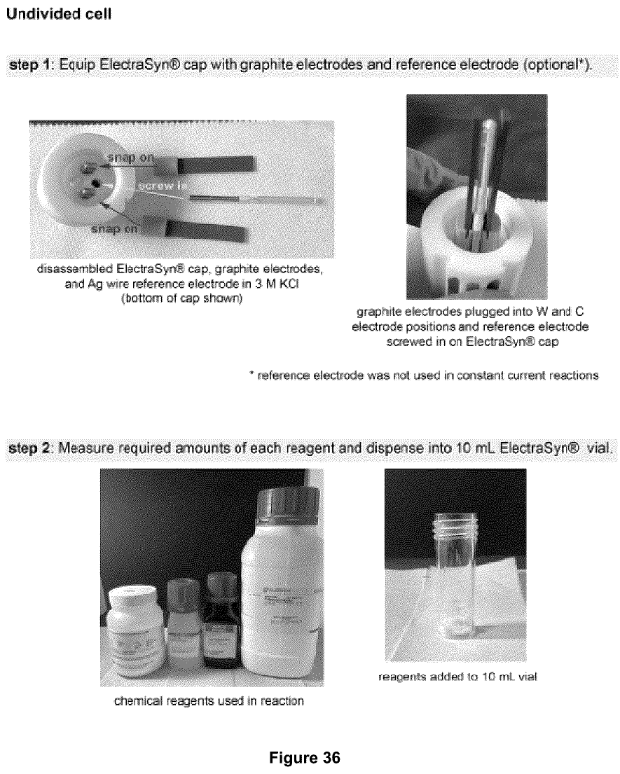

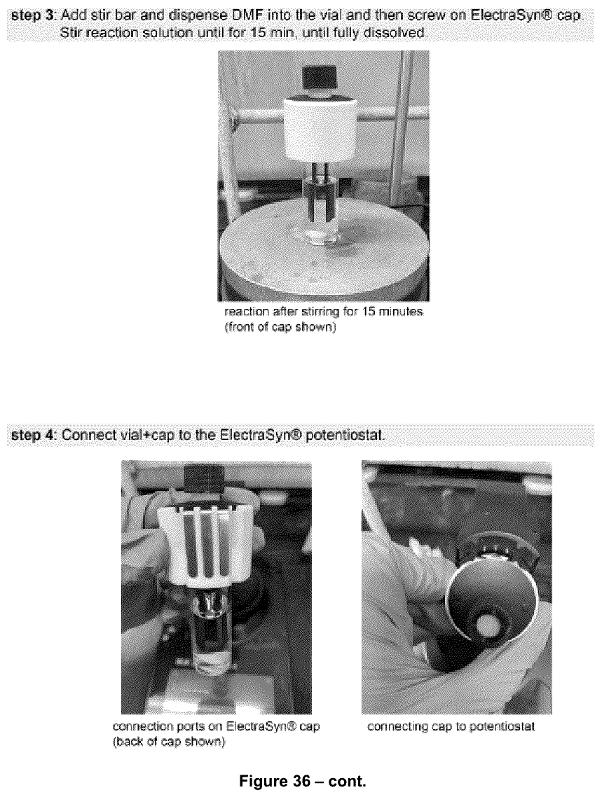

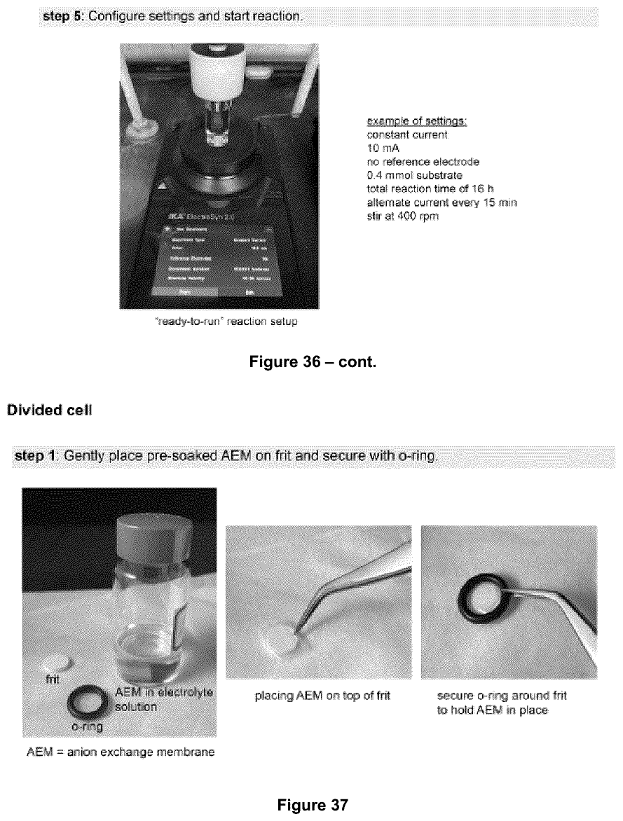

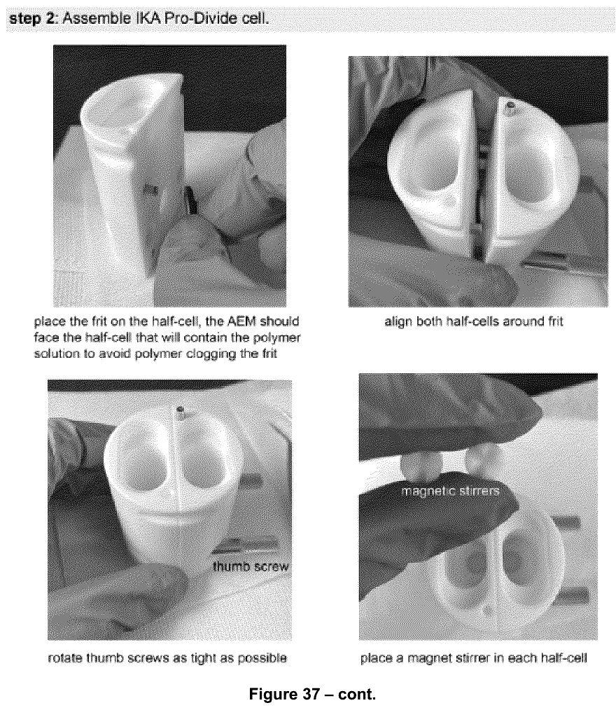

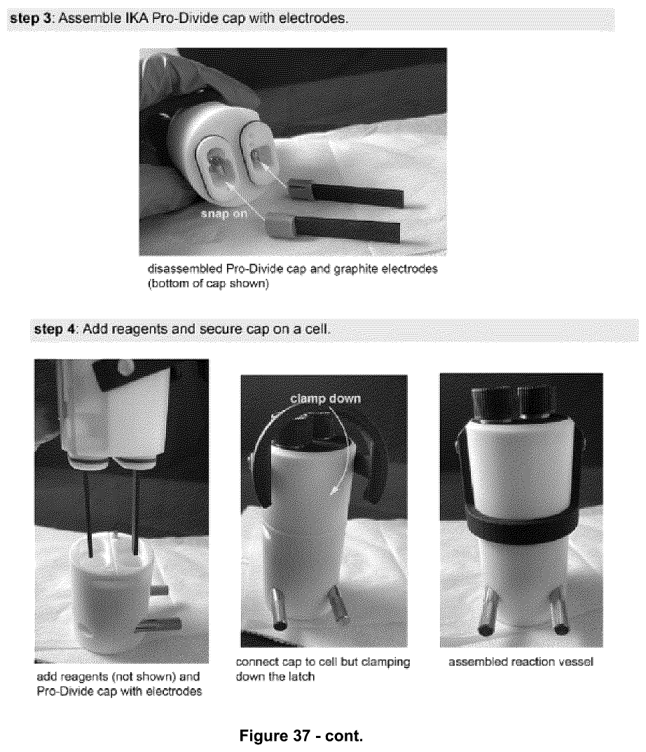

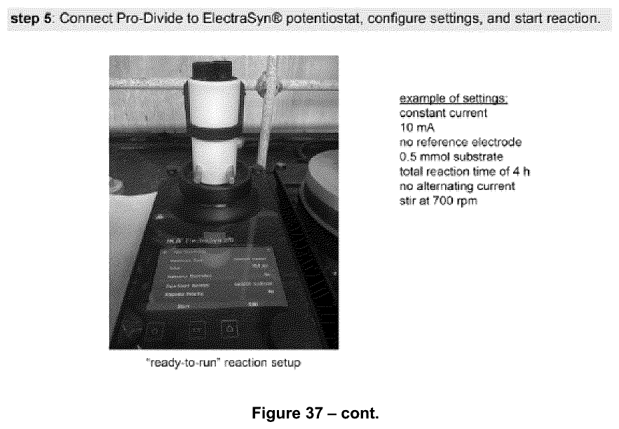

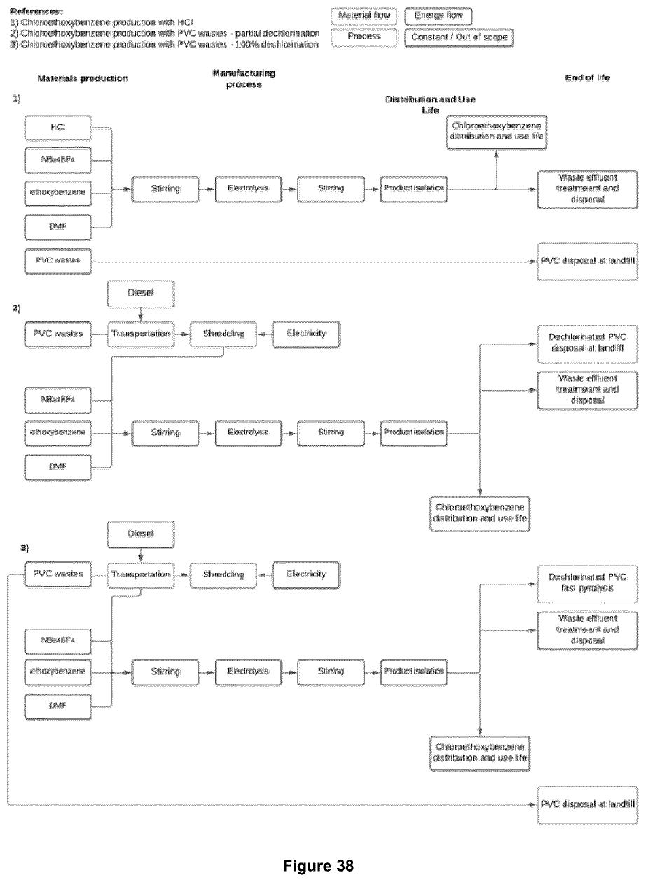

FIG. 1 provides a schematic illustration of a method in accordance with the disclosure. While FIG. 1A specifically references a PVC waste source, it should be understood that the schematic is generalizable to other halogen containing polymers. By products shown in FIG. 1A can vary depending on the waste source used and/or reaction conditions, for example. The electrolysis process can be performed in a divided or undivided cell. FIG. 36 illustrates a process flow for performing the method of the disclosure in an undivided cell. FIG. 37 illustrates a process flow for performing the method of the disclosure in a divided cell. Any suitable electrochemical cell arrangement can be used. The electrodes can be any known electrodes. For example, the electrodes can be formed of a material independently selected from graphite, gold, non-graphite carbon-based electrodes, and platinum. The electrodes can be the same or different materials. For example, the electrodes can both be graphite electrodes. Other forms of carbon-based electrodes can also be used. Platinum and/or gold electrodes can also be used.

A method for halogenation of an organic compound with a reactive halogen species generated from a waste source comprising a halogen-containing polymer can include admixing the waste source with the organic compound, a solvent, and an electrolyte, wherein the solvent swells and/or dissolves the halogen containing polymer in the waste source; and exposing the admixture to a negative voltage in an electrochemical cell comprising a cathode and an oppositely disposed anode. The halogen-containing polymer is dehalogenated by the cathode to generate a halogen anion. The halogen anion is formed into the reactive halogen species by the anode. The halogen species reacts with the organic compound to halogenate the organic compound.

A method for halogenation of an organic compound with a reactive halogen species from a waste source comprising a halogen containing polymer can include admixing the waste source with a solvent, and an electrolyte, wherein the solvent swells and/or dissolves the halogen containing polymer in the waste source. exposing the admixture to a negative voltage in an electrochemical cell comprising a cathode by which the halogen containing polymer is dehalogenated and halogen anions are generated, and an oppositely disposed anode by which the halogen anions are transformed into the reactive halogen species. The method can further include recovering the reactive halogen species from the electrochemical cell and admixing the reactive halogen species with the organic compound, wherein the reactive halogen species reacts with the organic compound to halogenate the organic compound.

In any of the methods of the disclosure, the waste source can be any source with a halogen-containing polymer. For example, the waste source can be poly(vinyl chloride). Mixed plastic waste sources can also be treated by the methods of the disclosure, where the method acts upon the halogen-containing polymer.

In a single-pot process, the waste source is admixed with the organic compound, an electrolyte, and a solvent. In a separated process, the waste source is admixed with the solvent and the solvent-treated waste source is then added to the electrochemical cell that has the organic compound and electrolyte. The admixture (whether including the organic compound or not) can further include a redox mediator in methods utilizing a redox mediator.

Methods in accordance with the disclosure can be mediator-free electrolysis processes in which the admixture is free of a redox mediator and no redox mediator is added during the process. Such methods can give high yields of chlorinated arenes and a black, insoluble char as the polymer product. The char has been observed to be substantially chlorine-free. The method can further include pyrolyzing or incinerating the char, which can be done safely as it is substantially chlorine free.

Methods in accordance with the disclosure can be free of added mediator. Free of added redox mediator refers to methods in which no redox mediator as an individual and distinct component is added in the admixture. The waste source, however, may contain components that can act as a redox mediator during the electrolysis.

The admixture (whether including the organic compound or not) is exposed to a negative voltage in an electrochemical cell containing a cathode and an oppositely disposed anode. The cell can be a divided or undivided cell. For some aromatic compounds that have reactivity at both the electrodes, a divided cell can be useful to isolate the aromatic compounds to maintain reactivity with only a single electrode. In a divided cell system, the admixture containing the waste source is introduced on cathode side of the divided cell, while the organic compound is introduced on the anode side of the divided cell.

The negative voltage can be −1V vs Ag/Ag⁺ or more negative. For example, the negative voltage can be about −1 V vs Ag/Ag⁺ to about −3 V vs Ag/Ag⁺. Other suitable voltages include −1, −1.2, −1.4, −1.6, −1.8, −2, −2.2, −2.4, −2.6, −2.8, or −3 V vs Ag/Ag⁺, or any values therebetween or ranges defined by such values.

The halogen containing polymer is dehalogenated by a reaction at the cathode to generate one or more halogen anions. Without intending to be bound by theory, it is believed that halogen anions generated during dehalogenation are formed into a reactive halogen species by the anode. This reactive halogen species then reacts with the organic compound to produce the halogenated organic compound. For example, with dechlorination of PVC, it is believed that Cl⁻ anions are oxidized to form Cl₂, which react with the organic compound to chlorinate the organic compound.

Based on this mechanistic understanding, it was determined that the process of halogenation can be physically separated from the dehalogenation. The reactive halogen species can be recovered from the electrochemical cell and transported to a compartment containing the organic compound to be halogenated. The compartment can be part of the electrochemical cell or physically separated therefrom.

The halogen of the halogen containing polymer can be Cl, F, I, or Br. Chlorinated polymers can include polyvinyl chloride and polyvinylidene dichloride. PVC having more chlorination as compared to standard PVC (generally referred to as cPVC) can also be treated by methods of the disclosure. Fluorinated polymers can include PVDF, PVF, and Teflon. Dechlorinated PVC is generally referred to herein as dPVC without regard to the degree or level of dechlorination.

The solvent can be any solvent that is capable of at least swelling the waste source. For example, the solvent can be selected as one capable of dissolving the waste source. For waste sources containing mixed plastics, the solvent can be selected to swell or dissolve the polymer having the halogen of interest for recovery. Other polymers within the mixed waste source may or may not be susceptible to swelling and/or dissolving by the solvent. The solvent can be selected to be stable in the reducing conditions of the method of the disclosure. For example, dimethylformamide (DMF) can be used as the solvent. Other solvents include acetonitrile, propylene carbonate, and dimethoxyethane. Mixtures of solvents can also be used.

Any electrolyte that is soluble in an organic solvent can be used for the electrochemical process. For example, the electrolyte can be one or more of tetrabutylammonium tetrafluoroborate, tetrabutylammonium hexafluorophosphate, tetrabutylammonium bis(trifluoromethanesulfonyl)imide.

The redox mediator, if present, can be an organic or organometallic compound that undergoes reduction at about −1 V versus Ag/Ag⁺ or more negative. For example, the redox mediator can under reduction at about −1.6V or more negative. Cyclic voltammetry can be used to screen redox active organic compounds to determine those suitable for the methods of the disclosure—i.e., those undergoing reduction at about −1V verses Ag/Ag⁺ or more negative. For example, the redox mediator can be one or more phthalates. For example, the redox mediator can be di-2-ethylhexyl phthalate (DEHP). For example, the redox mediator can be one or more of p-tolunitrile, terephthalonitrile, benzonitrile, pyridazine, 1, 10-phenanthroline, neocuproine, bathophenanthroline, phenanthridine, 2,2′-bipyridine, trans-stilbene, and p-terphenyl. For example, the redox mediator can be included in an amount of about 0 equiv to about 10 equiv, about 1 equiv to about 5 equiv, about 2 equiv to about 8 equiv, or about 0.5 equiv to about 3 equiv, relative to an amount of halogen containing polymer repeat unit present in the organic waste source. Other suitable amounts include about 0, 0.5, 1, 1.5, 2, 2.5, 3, 3.5, 4, 4.5, 5, 5.5, 6, 6.5, 7, 7.5, 8, 8.5, 9, 9.5 and 10 equiv, or any values therebetween or ranges defined by such values.

It has been observed that plasticizer present in the waste source can serve a redox mediator in some instances, and thereby eliminate the need for further addition of a redox mediator. For example, it was observed that the DEHP present in waste PVC served as a redox mediator in the processing of waste PVC. With this understanding, plastics additives can be engineered to not only have use in the primary manufacture of the plastic component itself, but also for use in downstream recycling processes, such as the methods of the disclosure.

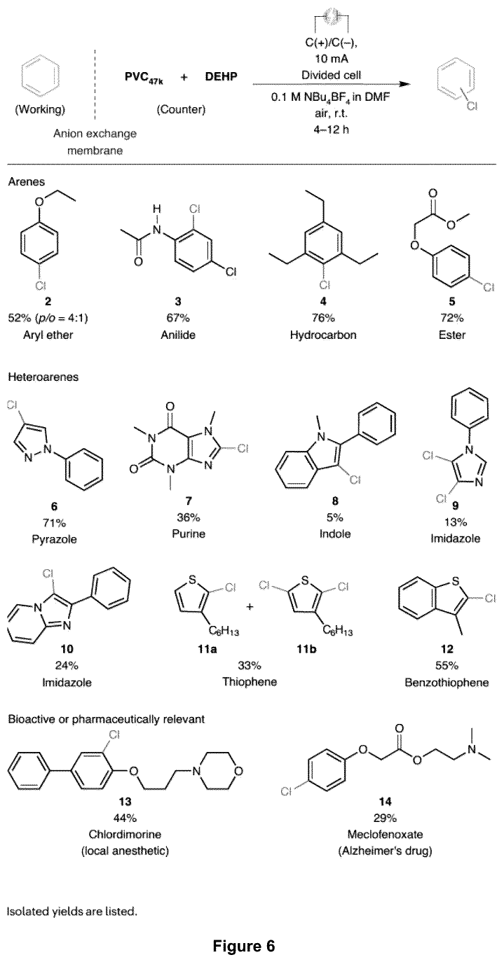

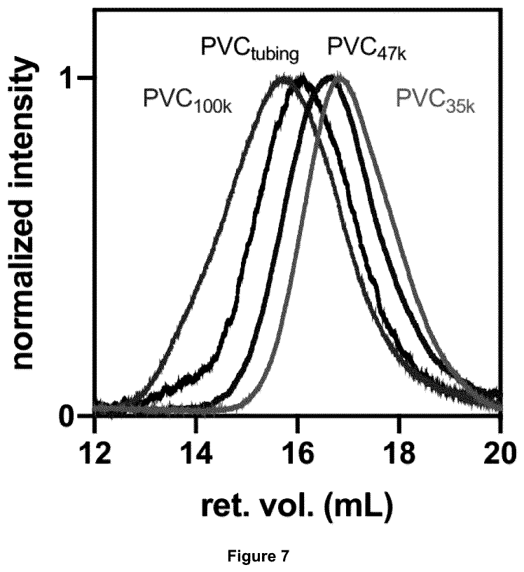

The organic compound can be any organic compound with unsaturation. For example, the organic compound can be an aromatic compound, alkenes, and alkynes. For example, the organic compound can be arenes. A divided-cell reaction set-up for various organic compounds to be halogenated is shown in FIG. 6. The reaction set-up is shown with PVC by way of example only. The methods of, the disclosure are not limited to this reaction step-up or the listed organic compounds.

The dehalogenated product resulting from the method of the disclosure can also have industrial or other applicability. For example, the decomposition of plasticizer in the waste source can leave the dehalogenated polymer as a clean polymer, which can be useful in other applications. In embodiments, the waste polymer itself can be transformed into a suitable compound for downstream use. For example, the method can be performed such that the halogen containing waste polymer is not full dehalogenated and/or otherwise reacted with other component to generate at least partially dehalogenated products having secondary use. For example, PVC can be partially dechlorinated and then resulting product can be converted to cPE via one or more additional reaction steps. For example, the additional reaction step can include dehydrogenation. Thus, the method can not only provide a source of halogen for halogenation of an organic compound but can also provide a useful product resulting from the dehalogenated or partially dehalogenated polymer, giving further use to the waste source.

### EXAMPLES

### Materials

Poly(vinyl chloride) (PVC) was obtained from Sigma Aldrich and Tygon Tubing®. Sigma Aldrich: PVC listed: Mw: ˜233,000, Mn: ˜99,000 (product #: 346764, lot #MKBW2191V), PVC listed: Mw: ˜80,000, Mn: ˜47,000 (product #: 389323, lot #MKCC7597), PVC listed: Mw: ˜43,000, Mn: ˜22,000 (product #: 389293, lot #MKCH4545). Tygon Tubing®: PVC tubing (Formulation: B-44-3, product #: 389293, lot #MKCH4545, inner diameter: ⅛-inch, outer diameter: ¼ inch, wall thickness: 1/16 inch.

The following reagents were used as received. From Sigma Aldrich: di(2-ethylhexyl) phthalate (lot #MKCK4506), ethoxybenzene, tridecane, 4′-chloroacetanilide, 1,3,5-triethylbenzene, caffeine, 2-chlorophenol, 4-chlorophenol, 4-phenylphenol, diisopropyl azodicarboxylate, triphenylphosphine, hydrochloric acid. From Oakwood Chemical: 1-phenylimidazole, 1-methyl-1H-indole, methyl phenoxyacetate, N,N-Dimethyl aminoethanol, (2,2,6,6-tetramethylpiperidin-1-yl)oxyl. From TCI: 3-methylbenzo[b]thiophene, 3-hexylthiophene, 2-phenyl[1,2-a]pyridine, 1-phenylpyrazole. From Fisher: triethylamine.

### Experimental Techniques

### Nuclear Magnetic Resonance (NMR) Spectroscopy

Unless otherwise noted, ¹H and ¹³C NMR spectra were acquired at rt. Chemical shift data are reported in units of δ (ppm) relative to tetramethylsilane (TMS). Spectra are referenced to residual solvent. Multiplicities are reported as follows: singlet (s), doublet (d), doublet of doublets (dd), triplet (t), quartet (q), multiplet (m).

### High-Resolution Mass Spectrometry (HRMS)

High-resolution mass spectrometry data were obtained on a Micromass AutoSpec Ultima Magnetic Sector mass spectrometer.

### Gas Chromatography-Mass Spectrometry (GC-MS)

GC-MS data was collected on Shimadzu GC-2010 gas chromatograph containing a Restek™ Rtx™-5 capillary columns with 15 m length, 0.25 mm inner diameter, 0.25 μm df (film thickness) with 5% diphenyl, 95% dimethsiloxane as the stationary phase, equipped with a Shimadzu GC-MS-QP2010S mass spectrometer. GC-MS data were analyzed using Shimadzu Corporation LabSolutions GC-MS solution Version 2.70 software. GC method: start and hold at 55° C. for 1 min, ramp 10° C./min to 270° C., hold at 270° C. for 10 min, total time=32.5 min.

### Sample Work-Up for GC-MS

A ˜0.05 mL aliquot of crude reaction mixture was diluted with ˜2 mL diethyl ether (Et2O) and filtered through a silica gel plug and a PTFE filter (0.2 μm) into a GC vial.

### Size-Exclusion Chromatography (SEC)

For SEC analysis, all polymers were dried under vacuum overnight, dissolved (˜0.5 mg polymer/mL) in THF spiked with trace toluene as a flow marker (<1 vol %), and filtered through a PTFE filter (0.2 μm). Polymer molar masses were determined by comparison with polystyrene standards (Varian, EasiCal PS-2 MW 580-377, 400) at 40° C. in THF on a Malvern Viscotek GPCMax VE2001 equipped with two Viscotek LT-5000L 8 mm (ID)×300 mm (L) columns and analyzed with Viscotek TDA 305, or on a Shimadzu LC-20AD equipped with two Styragel HT 7.8 mm (ID)×300 mm (L) columns and a PSS Gram column 8 mm (ID)×300 mm (L) and analyzed with Shimadzu SPD-M20A. Data presented corresponds to the refractive index (RI) response normalized to the highest peak, and data were obtained at a flow rate of 1 mL/min.

### Fourier-Transform Infrared (FTIR) Spectroscopy

FTIR spectroscopy data were obtained on neat samples using a Thermo-Nicolet IS-50 using the attenuated total reflectance (ATR) accessory.

### Elemental Analysis (EA)

EA was performed by Atlantic Microlab, Inc.

### Automatic Column Chromatography

Column chromatography was performed using a Biotage® Isolera™ One system and Biotage® Sfär Duo columns.

### Thermogravimetric Analysis (TGA)

Thermogravimetric analysis (TGA) thermograms were recorded on a TA Instruments Q50 TGA. All experiments were conducted on platinum TGA sample pans under a nitrogen purge of 50 mL/min with a heating rate of 10° C./min, covering a temperature range of 27° C. to 550° C. The instrument was calibrated using the Curie points of alumel and nickel standards.

### Differential Scanning Calorimetry (DSC)

DSC was performed under N₂ on a TA Instruments DSC Q2000 equipped with a TA RCS cooling accessory. Neat solid samples (˜3-5 mg) were dried under vacuum overnight and then placed in aluminum Tzero Low-Mass Hermetic pans and sealed with Tzero Hermetic lids using a TA Instruments crimper. Samples were cycled between −50° C. and 200° C. at a ramp rate of 5° C./min.

### Cyclic Voltammetry

Dimethylformamide, DMF (99.8%, anhydrous) was obtained from Sigma-Aldrich and used as received. Tetrabutylammonium tetrafluoroborate, NBu₄BF₄ (electrochemical grade) was obtained from Sigma-Aldrich and was dried under high vacuum for 48 h before being transferred to a nitrogen-filled glovebox. A 0.1 M stock solution of the supporting electrolyte in DMF was prepared in a nitrogen-filled glovebox (Mbraun Labmaster, water <4 ppm and oxygen <0.5 ppm) and was dried over 3 Å molecular sieves for at least two days before use.

Cyclic voltammetry was performed in a nitrogen-filled glovebox using a Biologic VSP multichannel potentiostat/galvanostat using a three-electrode electrochemical cell, consisting of a glassy carbon disk working electrode (3 mm diameter, 0.07 cm² area, BASi), an Ag/Ag⁺ quasi-reference electrode (BASi) with 0.01 M AgBF₄ (Sigma-Aldrich) in dimethylformamide calibrated with Fc/Fc⁺, and a platinum wire counter electrode (BASi). All experiments were conducted in a 0.1 M NBu₄BF₄ stock electrolyte solution

### Molar Mass of PVC Samples

The number-average (Mn) and weight-average (Mw) molar masses of each PVC source were measured by SEC. The measured values were different from the manufacturer listed values, and used to describe each PVC source herein, as highlighted in Table 1.

TABLE 1
List of PVC sources used in this work and the molar masses measured by SEC.

| | | measured Mn | measured Mw | dispersity, |
|---|---|---|---|---|
| PVC | Source and Product Info | (kg/mol) | (kg/mol) | Ð_{M} |
| PVC100k | Sigma Aldrich | 100 | 318 | 2.57 |
| | listed: Mw: ~233,000; Mn: ~99,000 | | | |
| PVC47k | Sigma Aldrich | 47 | 100 | 2.08 |
| | listed: Mw: ~80,000; Mn: ~47,000 | | | |
| PVC35k | Sigma Aldrich | 35 | 67 | 2.08 |
| | listed: Mw: ~43,000; Mn: ~22,000 | | | |
| PVCtubing, | Tygon Tubing | 83 | 191 | 2.42 |
| PVCsolid | | | | |

### Electrosynthesis Procedures

General Procedure A (undivided cell paired electrolysis, constant current, 3 mL scale) PVC (65 mg, 1.0 mmol (repeat unit), 8.0 equiv. (repeat unit)), NBu₄BF₄ (314 mg, 0.95 mmol), DEHP (0.050 mL, 0.13 mmol, 1.0 equiv.), ethoxybenzene (0.015 mL, 0.13 mmol, 1.0 equiv.), tridecane internal standard (0.010 mL), and DMF (3 mL) were added to a 5 mL ElectraSyn® reaction vessel equipped with Teflon coated magnetic stir bar (cylindrical, 12.7 mm length) and two graphite electrodes (0.8×0.2×5.20 cm). All reagents were dissolved by stirring for at least 15 min, then the reaction mixture was subjected to constant current electrolysis (3 or 7 mA, as specified in Table 2. Survey of current strength on a 3 mL scale paired-electrolysis reaction) with alternating polarity (15 min) at rt. The reaction was stirred at 400 rpm for 5-10 h.

General Procedure B (undivided cell paired electrolysis, constant voltage, 3 mL scale) PVC (65 mg, 1.0 mmol (repeat unit), 8.0 equiv. (repeat unit)), NBu₄BF₄ (314 mg, 0.95 mmol), DEHP (0.050 mL, 0.13 mmol, 1.0 equiv.), ethoxybenzene (0.015 mL, 0.13 mmol, 1 equiv.), tridecane internal standard (0.010 mL), and DMF (3 mL) were added to a 5 mL ElectraSyn® reaction vessel equipped with Teflon coated magnetic stir bar (cylindrical, 12.7 mm length), two graphite electrodes (0.8×0.2×5.20 cm), and a silver wire reference electrode stored in 3 M KCl in water. All reagents were dissolved by stirring for at least 15 min, then the reaction mixture was subjected to constant voltage electrolysis (−1.1, −1.2, −1.3, or −2.0 V, as specified in Table 3) with alternating polarity (15 min) at rt. The reaction was stirred at 400 rpm for 8 h.

General Procedure C (undivided cell paired electrolysis, constant current, 8 mL scale) PVC (200 mg, 3.2 mmol (repeat unit), 8.1 equiv. (repeat unit)), NBu₄BF₄ (264 mg, 0.80 mmol), DEHP (0.157 mL, 0.4 mmol, 1.0 equiv.), ethoxybenzene (0.051 mL, 0.4 mmol), tridecane internal standard (0.050 mL), and DMF (8 mL) were added to a 10 mL ElectraSyn® reaction vessel equipped with Teflon coated magnetic stir bar (egg-shaped, 16 mm length) and two graphite electrodes (0.8×0.2×5.20 cm). All reagents were dissolved by stirring for at least 15 min, then the reaction mixture was subjected to constant current electrolysis (10 mA) with alternating polarity (15 min) at rt. The reaction was stirred at 400 rpm for 16 h.

General Procedure D (undivided cell paired electrolysis, constant voltage, 8 mL scale) PVC (200 mg, 3.2 mmol (repeat unit), 8.1 equiv. (repeat unit)), NBu₄BF₄ (264 g, 0.80 mmol), DEHP (0.157 mL, 0.4 mmol, 1.0 equiv.), ethoxybenzene (0.4 mmol), tridecane internal standard (0.050 mL), and DMF (8 mL) were added to a 10 mL ElectraSyn® reaction vessel equipped with Teflon coated magnetic stir bar (egg-shaped, 16 mm length), two graphite electrodes (0.8×0.2×5.20 cm), and a silver wire reference electrode stored in 3 M KCl in water. All reagents were dissolved (or swelled) by stirring for at least 15 min, then the reaction mixture was subjected to constant voltage electrolysis (−1.3 V) with alternating polarity (15 min) at rt. The reaction was stirred at 400 rpm for 16 h.

### Bulk Electrolysis

Bulk electrolysis was performed using an ElectraSyn 2.0 and electrodes supplied by IKA. Reactions performed under N₂ were first degassed by bubbling N₂ for 30 min, and then electrolysis was performed under a N₂-filled balloon. All other reactions were performed opened to air. Anhydrous DMF (Sigma-Aldrich, lot no. SHBL1229) was stored in a sure/seal bottle and used in reactions performed under N₂. American Chemical Society reagent-grade DMF (Sigma-Aldrich, lot no. SHBD7622V) was used as received in reactions performed open to air. NBu₄BF₄ (Oakwood Chemical, lot no. 007316L07W) was used as electrolyte in all reactions. Undivided cell reactions were performed using a 10 ml IKA ElectraSyn vial. Divided-cell reactions were performed using an IKA Pro-Divide and a 5 μm frit. A FuelCell-Store PiperION anion-exchange membrane, 15 μm, mechanically reinforced, was pre-soaked in 0.1 M NBu4BF4 overnight and layered with the frit.

Bulk electrolysis (charging) was performed in a H-cell, with VitraPore frit (10 mm×2.8 mm×0.7 mm, porosity 5). The working and counter electrodes were reticulated vitreous carbon 5.1 cm long (McMaster 100 PPI 0.64×15.2×15.2 cm) and the reference electrode (Ag/Ag⁺) was used in the working side. The cycling current was set to −5 mA under a voltaic cutoff (−3.2 V or −4.0 V) and both chambers stirred at 700 r.p.m. during charging. The working chamber was loaded with active species _{(PVC47k} 0.125 g (8 equiv.) or DEHP 99 μl (1 equiv.) or PVC₄₇ₖ 0.125 g (8 equiv.)+DEHP 99 μl (1 equiv.)) in 0.1 M NBu₄BF₄ in DMF (5 ml). The counter chamber counter chamber was loaded with ethoxybenzene 29 μl (1 equiv.) in 0.1 M NBu₄BF₄ in DMF (5 ml).

### Example 1: Current Strength Analysis

The reactions were set up according to General Procedure A and monitored by GC-MS. Details on the GC-MS analysis are described at the end of this section. As shown in FIG. 11, once DEHP is consumed, the paired oxidative chlorination reaction tapers off. This information suggests that the rates of each half reaction (reduction and oxidation) should be matched. Note, the effect of operating currents varies with reaction scale. For example, the influence of 7 mA on a 3 mL reaction is different than 7 mA would be a on a larger scale. In forthcoming reactions on a larger scale (8 mL), 10 mA current was used.

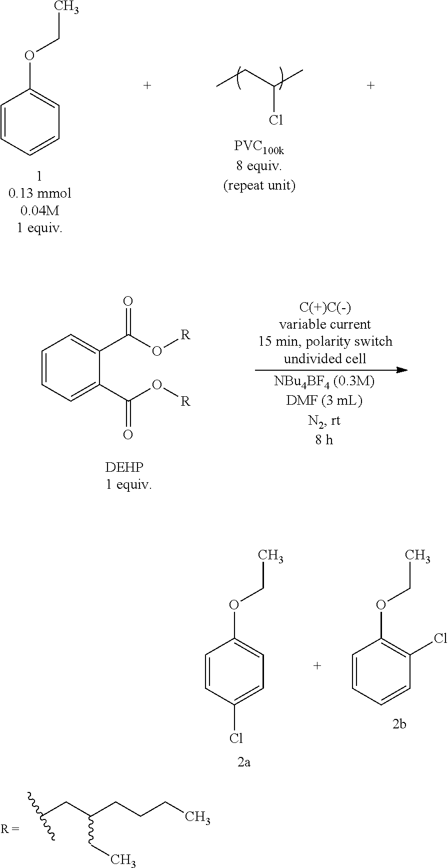

TABLE 2
Survey of current strength on a 3 mL
scale paired-electrolysis reaction.

| | | conversion of 1 | consumption of | yield of 2 |
|---|---|---|---|---|
| entry | conditions | (%) | DEHP (%) | (%) |
| 1 | 3 mA, 10 h | 67 | 89 | 53 |
| 2 | 7 mA, 5 h | 56 | 95 | 35 |

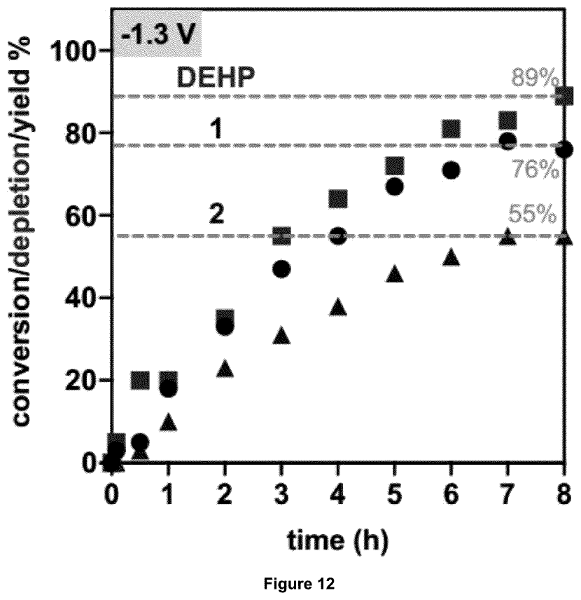

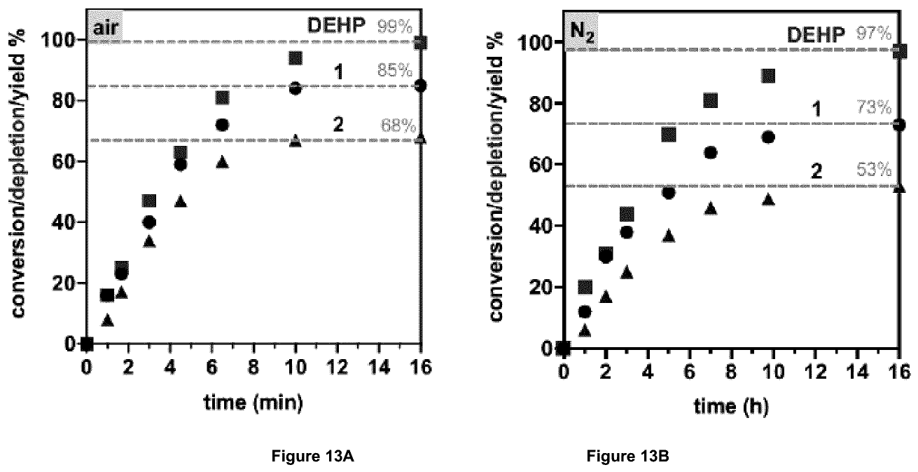

FIG. 12 shows the monitoring of the conversion of compound 1, consumption of DEHP, and the yield of compound 2 over time under 3 mA (left) and 7 mA (right) constant current. The reactions were performed on a 3 mL scale under N₂.

### Example 2: Voltage Analysis

The reactions were set up according to General Procedure B and monitored by GC-MS. −1.3 V is the smallest voltage at which the reaction proceeds and is used in forthcoming constant voltage experiments. Note, this voltage is achieved using the potentiostat built into the IKA ElectraSyn, which differs from the potentiostat used in the cyclic voltammetry and bulk electrolysis studies.

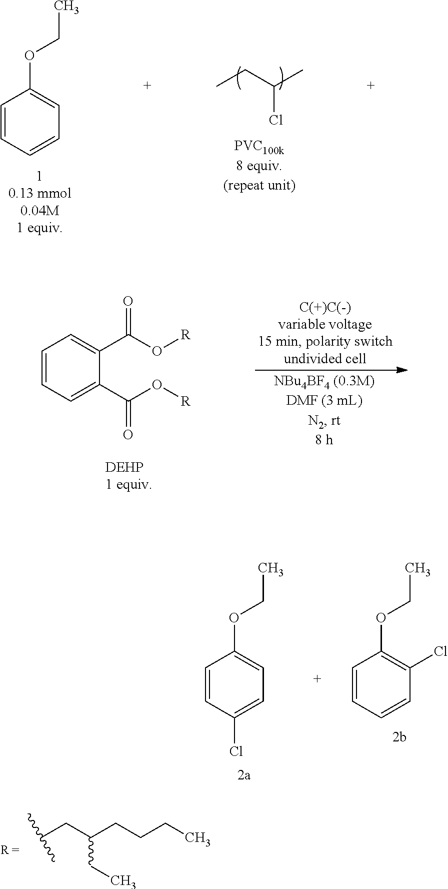

TABLE 3
Survey of voltage strength on a 3 mL
scale paired-electrolysis reaction.

| | | conversion of 1 | consumption of | yield of 2 |
|---|---|---|---|---|
| entry | conditions | (%) | DEHP (%) | (%) |
| 1 | −1.1 ᵥa | n.r. | n.r. | n.r. |
| 2 | −1.2 ᵥa | n.r. | n.r. | n.r. |
| 3 | −1.3 V, 8 h | 76 | 89 | 55 |
| 4 | −2.0 V^{b}, 2 h | 43 | 44 | 0 |

aReaction auto shutdown in less than 15 min because the resistance was too high.
^{b}Reaction was turned off after 2 h because no product was observed although starting material was being consumed.
n.r. = no reaction occurred

FIG. 12 shows the monitoring of the conversion of compound 1, the consumption of DEHP, and the yield of 2 over time under −1.3 V constant voltage. The reaction was performed on a 3 mL scale under N₂.

### Example 3: Comparison of Air vs. N₂

The reactions were set up according to General Procedure C and monitored by GC-MS. Each set of conditions were run in duplicate. This data shows that this reaction proceeds to higher yields under air.

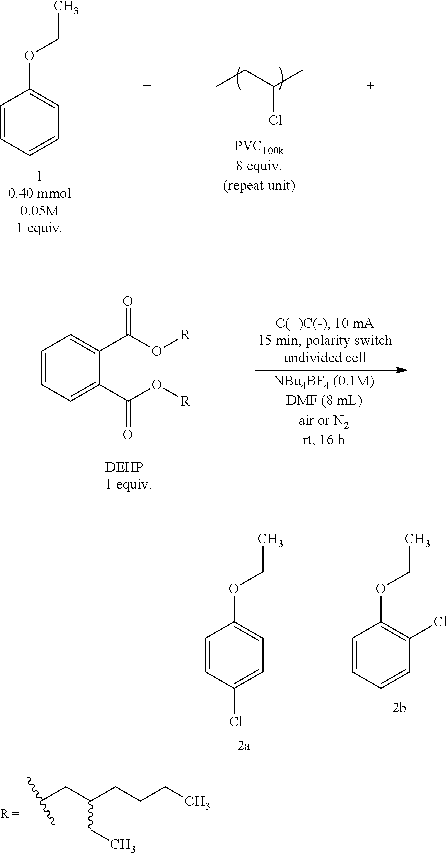

TABLE 4
Survey of air versus N₂ atmosphere on
the paired-electrolysis reaction.

| | | conversion of 1 | consumption of | yield of 2 |
|---|---|---|---|---|
| entry | conditions | (%) | DEHP (%) | (%) |
| 1 | wet^{a} DMF | 85, 93 | 99, 100 | 68, 77 |
| | air | | | |
| 2 | anhydrous^{b} DMF | 73, 79 | 97, 98 | 53, 54 |
| | N₂ | | | |

^{a}“Wet” DMF was stored in an opened bottle
^{b}“Anhydrous” DMF was stored in sure/seal bottle

FIG. 13 show the monitoring of the conversion, the consumption of DEHP and the yield of 2 over time under air (left) and a N₂ atmosphere (right). The reaction was performed on an 8 mL scale.

### Example 4: Analysis of PVC Molecular Weight

The reactions were set up according to General Procedure C and monitored by GC-MS. Each set of conditions were run in duplicate. Tributylamine was only observed in the GC-MS traces of reactions performed without DEHP.

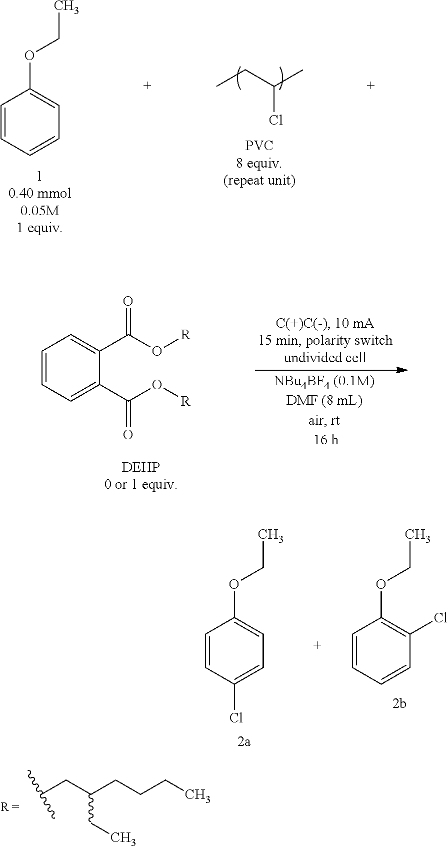

TABLE 5
Survey of effect of PVC molecular weight and
presence of DEHP under constant current.

| | | PVC | DEHP | Conversion | Consumption | Yield | |
|---|---|---|---|---|---|---|---|
| Entry | PVC Source | Equiv. | Equiv. | of 1 (%) | of DEHP (%) | of 2 (%) | NBu₃ |
| 1 | PVC100k | 8 | 0 | 53, 51 | n/a | 26, 31 | y, y |
| 2 | PVC100k | 8 | 1 | 85, 93 | 99, 100 | 68, 77 | n, n |
| 3 | PVC47k | 8 | 0 | 76, 71 | n/a | 52, 59 | y, y |
| 4 | PVC47k | 8 | 1 | 97, 99 | 96, 99 | 78, 92 | n, n |
| 5 | PVC35k | 8 | 0 | 95, 100 | n/a | 86, 89 | y, y |
| 6 | PVC35k | 8 | 1 | 99, 99 | 94, 97 | 74, 75 | n, n |

n/a = not applicable because reagent was not used in this reaction

### Example 5: Effect of PVC Molecular Weight and DEHP Equivalents Under Potentiostatic Conditions

The reactions were set up according to General Procedure D and monitored by GC-MS. Each set of conditions were run in duplicate. Reactions auto shutdown when there was no DEHP present in the reaction mixture because the resistance was too high. Tributyl amine was observed in the GC-MS traces of all reactions where conversion is observed.

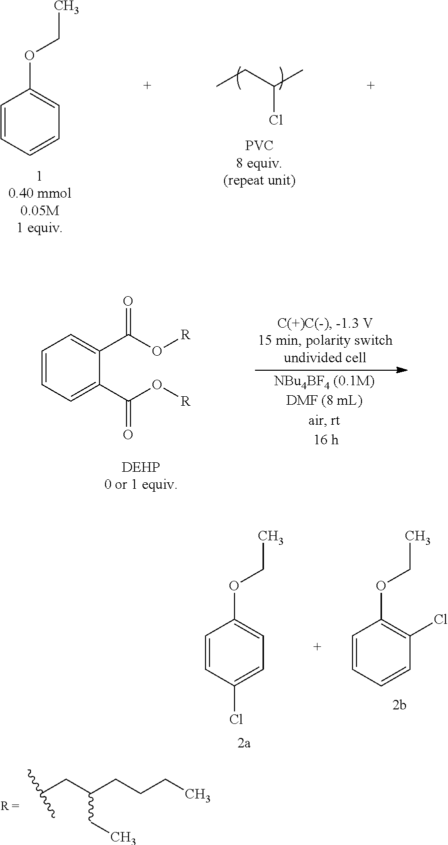

TABLE 6
Survey of effect of PVC molecular weight and
presence of DEHP under constant voltage.

| | | PVC | DEHP | conversion | consumption | yield | |
|---|---|---|---|---|---|---|---|
| Entry | PVC Source | equiv. | equiv. | of 1 (%) | of DEHP (%) | of 2 (%) | NBu3 |
| 1 | PVC100k | 8 | 0 | n.r. | n/a | n.r. | n.r. |
| 2 | PVC100k | 8 | 1 | 84, 73 | 83, 77 | 54, 41 | y, y |
| 3 | PVC47k | 8 | 0 | n.r. | n/a | n.r. | n.r. |
| 4 | PVC47k | 8 | 1 | 88, 81 | 98, 94 | 57, 46 | y, y |
| 5 | PVC35k | 8 | 0 | n.r. | n/a | n.r. | n.r. |
| 6 | PVC35k | 8 | 1 | 73, 82 | 94, 99 | 36, 48 | y, y |

n.r. = no reaction occurred
n/a = not applicable because reagent was not used in this reaction

### GC-MS Analysis

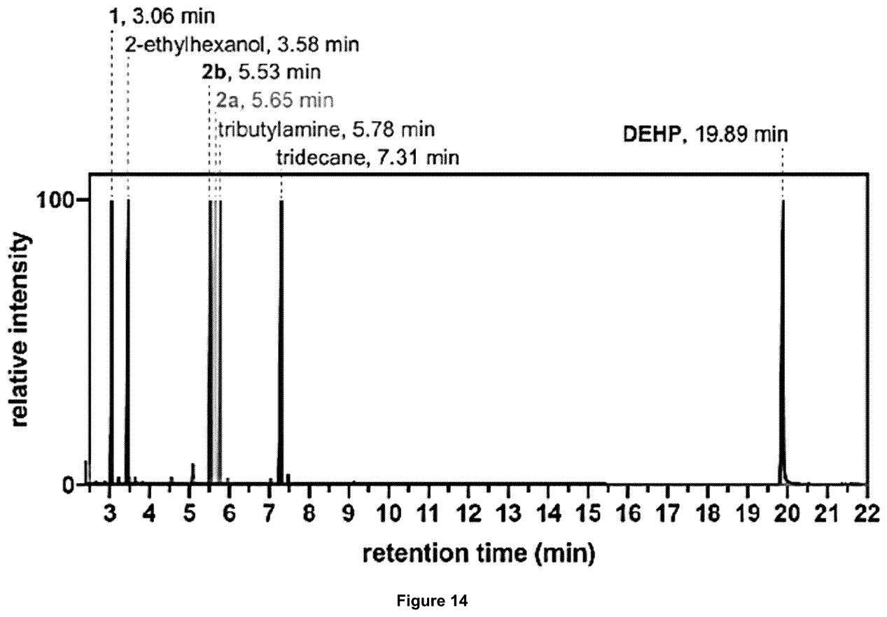

Referring to FIG. 14, GC-MS data was collected for several chemical standards and used as references for retention times. The mass spectrum of each sample matched >90% with spectra in Shimadzu mass-spec library.

GC-MS response factors of products 4-chloro-1-ethoxybenzene (2a) and 4-chloro-1-ethoxybenzene (2b) vs. starting material ethoxybenzene (1) were calculated using the molar response measured from the GC peak areas of known solution concentrations (eq. 1).

$$response factor = \frac{molar response of 2 a or 2 b}{molar response of 1} = \frac{area / concentration}{area / concentration} \qquad ( eq . 1 )$$

Six solutions of known concentrations were prepared according to volumes listed in the table below. For each, the indicated amount of analyte 1, 2a, or 2b was dispensed from a micropipette into a 25 mL volumetric flask and then diluted with acetone. Two GC-MS traces were taken for each solution, generating 12 total molar response data points for each analyte. These data points were used calculate average response factors for 2a vs. 1 and 2b vs. 1.

$$response factor of 2 a vs . 1 = 1.17 \pm 0 \text{.07}$$

$$response factor of 2 b vs . 1 = 1.16 \pm 0.05$$

TABLE 7
Data used to calculate GC-MS response factors, including sample
preparation measurements and data extracted from chromatograms.

| | | Vol. | | | | | |
|---|---|---|---|---|---|---|---|
| | | added | concentration | | GC peak | Molar | Response |
| Solution | Analyte | (μL) | (M)^{a} | Trial | area | response | factor vs. 1 |
| 1 | 1 | 10 | 0.00315 | 1 | 6753561 | 2146416616 | 1.00 |
| | | | | 2 | 7862605 | 2498892957 | 1.00 |
| | 2a | 10 | 0.00286 | 1 | 6660478 | 2327511290 | 1.08 |
| | | | | 2 | 7598539 | 2655317728 | 1.06 |
| | 2b | 10 | 0.00288 | 1 | 6923597 | 2401454036 | 1.12 |
| | | | | 2 | 7875972 | 2731785912 | 1.09 |
| 2 | 1 | 12 | 0.00378 | 1 | 8167544 | 2163173887 | 1.00 |
| | | | | 2 | 7462957 | 1976564032 | 1.00 |
| | 2a | 12 | 0.00343 | 1 | 9236690 | 2689809482 | 1.24 |
| | | | | 2 | 8002472 | 2330393795 | 1.18 |
| | 2b | 12 | 0.00346 | 1 | 8717753 | 2519798491 | 1.17 |
| | | | | 2 | 7684397 | 2221115001 | 1.12 |
| 3 | 1 | 14 | 0.00441 | 1 | 6785241 | 1540346538 | 1.00 |
| | | | | 2 | 7603729 | 1726154995 | 1.00 |
| | 2a | 14 | 0.00401 | 1 | 7755426 | 1935815758 | 1.26 |
| | | | | 2 | 8100810 | 2022026340 | 1.17 |
| | 2b | 14 | 0.00404 | 1 | 7443360 | 1844095831 | 1.20 |
| | | | | 2 | 8026028 | 1988452094 | 1.15 |
| 4 | 1 | 16 | 0.00503 | 1 | 10251061 | 2036244996 | 1.00 |
| | | | | 2 | 11508964 | 2286111687 | 1.00 |
| | 2a | 16 | 0.00458 | 1 | 10215492 | 2231134252 | 1.10 |
| | | | | 2 | 11783768 | 2573656599 | 1.13 |
| | 2b | 16 | 0.00461 | 1 | 10468225 | 2269319074 | 1.11 |
| | | | | 2 | 12033682 | 2608681423 | 1.14 |
| 5 | 1 | 18 | 0.00568 | 1 | 11447268 | 2021205821 | 1.00 |
| | | | | 2 | 10998116 | 1941900555 | 1.00 |
| | 2a | 18 | 0.00515 | 1 | 13374301 | 2596480026 | 1.29 |
| | | | | 2 | 12206113 | 2369688599 | 1.22 |
| | 2b | 18 | 0.00519 | 1 | 13184267 | 2540539677 | 1.26 |
| | | | | 2 | 11978904 | 2308272496 | 1.19 |
| 6 | 1 | 20 | 0.00629 | 1 | 10923164 | 1735799878 | 1.00 |
| | | | | 2 | 9146106 | 1453407609 | 1.00 |
| | 2a | 20 | 0.00572 | 1 | 11357821 | 1984501458 | 1.14 |
| | | | | 2 | 10118519 | 1767963741 | 1.22 |
| | 2b | 20 | 0.00577 | 1 | 11581444 | 2008515619 | 1.16 |
| | | | | 2 | 10135133 | 1757688673 | 1.21 |

^{a}Concentrations were calculated for a total volume of 25 mL. The densities of 1, 2a, and 2b (0.961, 1.1204, and 1.1288 g/mL, respectively) were taken from the literature and used to calculate concentration.¹ The molecular weights of 1, 2a, and 2b (122.17, 156.61, and 156.61 g/mol, respectively) were also used to calculate concentration

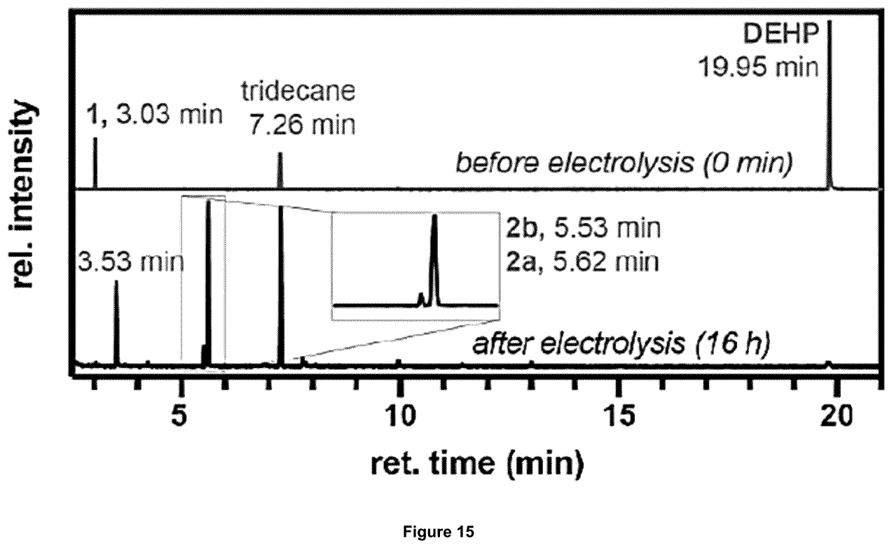

For each reaction, GC-MS was taken before electrolysis (0 min) and after electrolysis (typically 16 h). Representative chromatograms and peak integration area data is shown in FIG. 15. Tridecane was used as an internal standard.

TABLE 8
Raw data extracted from representative GC-MS
chromatograms from 10 mA constant current electrolysis
with PVC47k and 1 equiv. of DEHP.

GC-MS peak area

| compound | 0 min | 16 h |
|---|---|---|
| 1 | 2295449 | 25293 |
| 2b | 0 | 264078 |
| 2a | 0 | 2126567 |
| tributyl amine | 0 | 0 |
| tridecane | 2061868 | 2007309 |
| DEHP | 12497710 | 107637 |

The fraction of each analyte (1, 2a, 2b, and DEHP) relative to the internal standard (tridecane) was calculated for each using the following equation:

$$fraction relative to standard = \frac{{area}_{analyte}}{{area}_{internal standard}} \qquad ( eq . 2 )$$

TABLE 9
Representative GC-MS data normalized
to tridecane internal standard.

fraction relative to
standard

| compound | 0 min | 16 h |
|---|---|---|
| 1 | 1.113 | 0.013 |
| DEHP | 6.061 | 0.054 |
| 2b | 0.000 | 0.132 |
| 2a | 0.000 | 1.059 |

The percent of 1 converted during electrolysis was determined using the following equation:

$$\% of 1 converted = ( 1 - \frac{fraction of 1 at 16 h}{fraction of 1 at 0 min} ) \times 100 \% \qquad ( eq . 3 )$$

The percent of DEHP consumed during electrolysis was determined using the following equation:

$$\% of DEHP consumed = ( 1 - \frac{fraction of DEHP at 16 h}{fraction of DEHP at 0 min} ) \times 100 \% \qquad ( eq . 4 )$$

The relative percent of 2a generated during electrolysis (% yield) was determined using the following equation, in which 1.17 is used as the response factor for 2a vs. 1:

$$= \% yield of 2 a = ( \frac{fraction of 2 a at 16 h}{1.17 * fraction of 1 at 0 min} ) \times 100 \% \qquad ( eq . 5 )$$

The relative percent of 2b generated during electrolysis (% yield) was determined using the following equation, in which 1.16 is included as the response factor for 2b vs. 1:

$$\% yield of 2 b = ( \frac{fraction of 2 b at 16 h}{1.16 * fraction of 1 at 0 min} ) \times 100 \% \qquad ( eq . 6 )$$

The total yield of 2 was determined by adding the individual yields of 2a and 2b.

$$total \% yield of 2 = \% yield of 2 a + \% yield of 2 b \qquad ( eq . 7 )$$

TABLE 10
Final values determined from representative GC-MS data from 10 mA
constant current electrolysis with PVC47k and 1 equiv. of DEHP.

% converted, consumed, or
generated

| compound | 0 min | 16 h |
|---|---|---|
| 1 | 0 | 99 |
| DEHP | 0 | 99 |
| 2b | 0 | 10 |
| 2a | 0 | 81 |
| total yield of 2 | — | 92 |

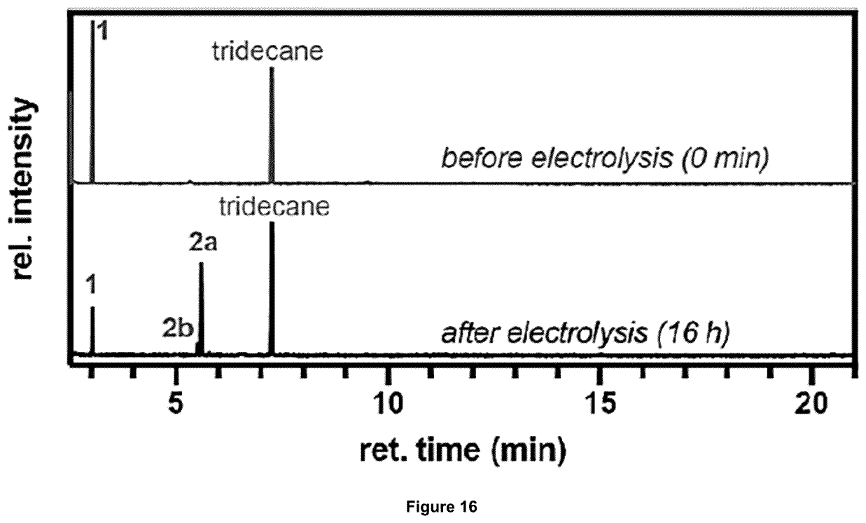

FIG. 16 shows representative GC-MS collected before and after the electrochemical reaction performed according to General Procedure C, using PVC47k and 0 equiv. of DEHP under 10 mA constant current. Note, no material eluted after 21 min.

TABLE 11
Raw data extracted from representative GC-MS chromatograms from
10 mA constant current electrolysis with PVC47k and no DEHP.

GC-MS peak area

| compound | 0 min | 16 h |
|---|---|---|
| 1 | 3332164 | 541219 |
| 2b | 0 | 168956 |
| 2a | 0 | 1178574 |
| tributyl amine | 0 | 40544 |
| tridecane | 2818302 | 1872548 |
| DEHP | 0 | 0 |

TABLE 12
Final values determined from representative GC-MS data from
10 mA constant current electrolysis with PVC47k and no DEHP.

% converted, consumed, or
generated

| compound | 0 min | 16 h |
|---|---|---|
| 1 | 0 | 76 |
| DEHP | — | — |
| 2b | 0 | 7 |
| 2a | 0 | 45 |
| total yield of 2 | — | 52 |

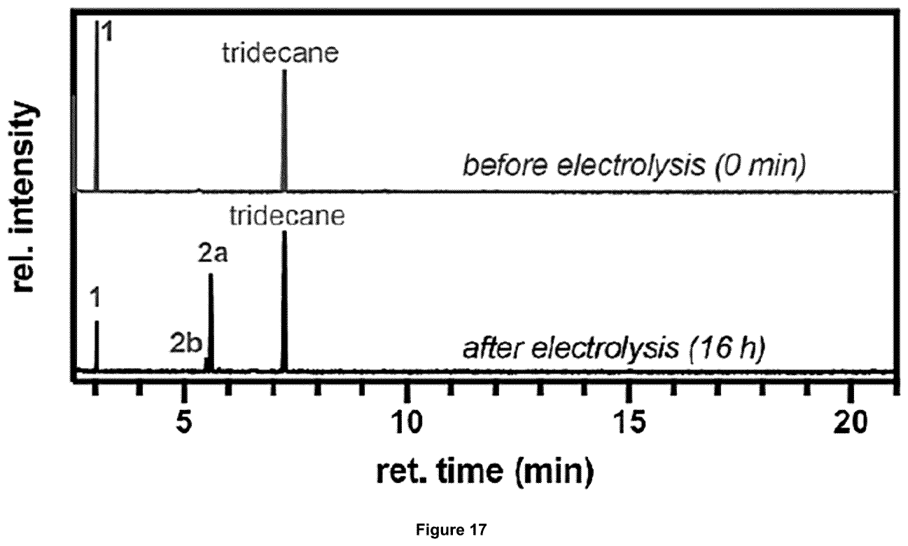

FIG. 17 shows representative GC-MS collected before and after the electrochemical reaction performed according to general procedure D, using PVC47k and 1 equiv. of DEHP under −1.3 V constant voltage. Note, no material eluted after 21 min.

TABLE 13
Raw data extracted from representative GC-MS
chromatograms from −1.3 V constant voltage
electrolysis with PVC47k and 1 equiv. DEHP.

GC-MS peak area

| compound | 0 min | 16 h |
|---|---|---|
| 1 | 2675738 | 418466 |
| 2b | 0 | 123465 |
| 2a | 0 | 1064898 |
| tributyl amine | 0 | 926672 |
| tridecane | 2178672 | 1793189 |
| DEHP | 10665027 | 521906 |

TABLE 14
Final values determined from representative GC-MS data from −1.3
V constant voltage electrolysis with PVC47k and 1 equiv. DEHP.

% converted, consumed, or
generated

| compound | 0 min | 16 h |
|---|---|---|
| 1 | 0 | 81 |
| DEHP | 0 | 94 |
| 2b | 0 | 5 |
| 2a | 0 | 41 |
| total yield of 2 | — | 46 |

### Example 7: Bulk Electrolysis

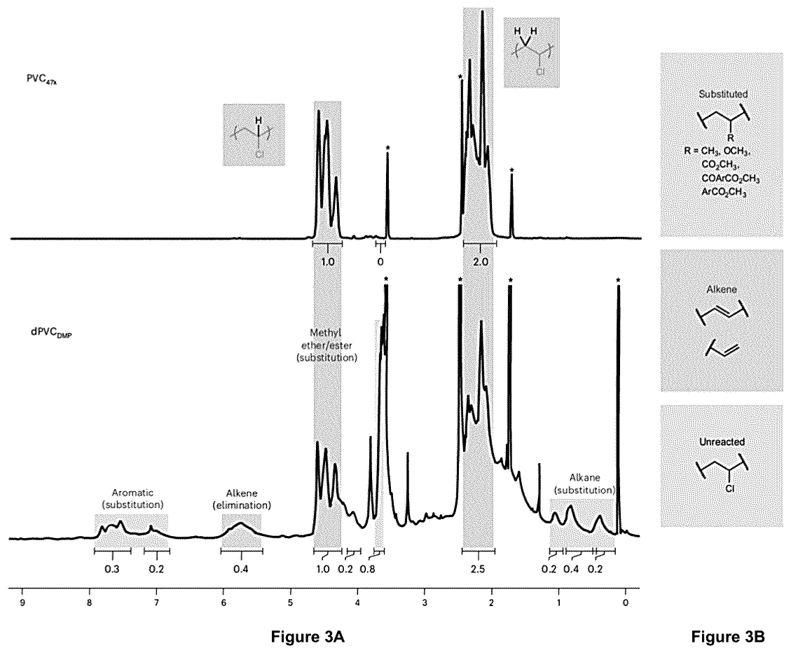

Bulk electrolysis under constant current was performed to monitor the voltage readout of the reaction. After constant current bulk electrolysis, each half-reaction was analyzed by GC-MS. The voltages (vs. Ag/Ag⁺) were read at capacity 0 mA*h, see FIG. 3B. The theoretical capacity of 5 mL of a 50 mM DEHP solution (0.25 mmol) was calculated to be 6.70 mA*h using the following conversion, in which 96485 C/mol is the Faraday constant.

$$0.25 mmol DEHP * \frac{1 mol}{1000 mmol} * \frac{96485 C}{1 mol} * \frac{1 A * s}{1 C} * {\frac{1000 mA}{1 A *}}_{3600 s = 6.7 mA * h}^{1 h}$$

### Example 6: Pair Electrolysis Using PVC as the Chloride Source

Referring to FIGS. 1A and 1B, the paired-electrolysis process of the disclosure was performed in an undivided cell using ethoxybenzene as the organic compound. Dimethylformamide (DMF) was selected as the solvent as it has a wide electrochemical window and dissolves (or swells) the reaction components. The electrodes were both graphite. Having identical electrodes enabled alternation of the polarity to minimize polymer build-up on the electrodes. Tetrabutylammonium tetrafluoroborate (NBu₄BF₄) was chosen as the electrolyte so that the non-nucleophilic anion would not interfere in the synthetic reactions. Three different number average molar masses (Mₙ) of PVC were used: PVC_{35K}, PVC₄₇ₖ, and PVC_{100K}.

The two lower-molar-mass polymers completely dissolved in DMF at room temperature, whereas PVC_{100K} only partially dissolved and swelled. Reaction screening was first performed without DEHP present.

The following conditions were used for the undivided cell paired-electrolysis, constant-current, 8 ml scale: PVC (200 mg, 3.2 mmol (repeat unit), 8.1 equiv. (repeat unit)), NBu4BF4 (264 mg, 0.80 mmol), DEHP (0.157 ml, 0.4 mmol, 1.0 equiv.), ethoxybenzene (0.051 ml, 0.4 mmol), tri-decane internal standard (0.050 ml) and DMF (8 ml) were added to a 10 ml ElectraSyn reaction vessel equipped with Teflon coated magnetic stir bar (egg-shaped, 16 mm length) and two graphite electrodes (0.8×0.2×5.20 cm). All reagents were dissolved by stirring for at least 15 min, then the reaction mixture was subjected to constant-current electrolysis (10 mA) with alternating polarity (15 min) at room temperature. The reaction was stirred at 400 r.p.m. for 16 h

Under constant-current electrolysis (galvanostatic conditions at 10 mA), the chlorination reaction proceeded to higher yields with the lower-molar-mass PVC (88% for PVC_{35K}), even though the total mass of the polymer added was consistent in each case (FIG. 1C). This molar mass dependency may be due to the increased diffusivity of smaller macromolecules compared with larger ones. Conformational differences may also play a role, wherein the larger macromolecules are more coiled and therefore have less accessible C—Cl bons. Furthermore, at the same mass loading, the lower-molar-mass polymers have more individual chains leading to more frequent collisions with the electrode. With all PVC sources, a build-up of insoluble black residue was observed on the electrodes after the reaction, suggesting that PVC was being reduced at the electrode surface.

### Example 7: Paired-Electrolysis Using PVC and Plasticizer

The same constant-current paired-electrolysis described in Example 6 were performed with DEHP present. The reactions with PVC_{47K} and PVC₁₀₀ₖ proceeded to higher yields (85% and 73%, respectively), whereas the already high yield using PVC_{35K} decreased slightly to 75%. Little-to-no insoluble polymer residue was observed on the electrodes, suggesting that PVC was being reduced indirectly rather than directly at the electrode surface.

The same reactions were performed under constant-voltage conditions (potentiostatic conditions at −1.3 V verse Ag/AgCl), which was the smallest voltage that showed reactivity using an IKA ElectraSyn. The following conditions were used for the undivided cell paired-electrolysis, constant-voltage, 8 ml scale: PVC (200 mg, 3.2 mmol (repeat unit), 8.1 equiv. (repeat unit)), NBu₄BF₄ (264 g, 0.80 mmol), DEHP (0.157 ml, 0.4 mmol, 1.0 equiv.), ethoxybenzene (0.4 mmol), tri-decane internal standard (0.050 ml) and DMF (8 ml) were added to a 10 ml ElectraSyn reaction vessel equipped with Teflon coated magnetic stir bar (egg-shaped, 16 mm length), two graphite electrodes (0.8×0.2×5.20 cm) and a silver wire reference electrode stored in 3 M KCl in water. All reagents were dissolved (or swelled) by stirring for at least 15 min, then the reaction mixture was subjected to constant-voltage electrolysis (−1.3 V) with alternating polarity (15 min) at room temperature. The reaction was stirred at 400 r.p.m. for 16 h.

When DEHP was included, the chlorination reaction proceeded but did not depend on Mₙ. Yield was about 42-52%. By contrast, no reaction was observed without DEHP (0% yield). The on/off switch in reactivity, with and without DEHP, indicates that PVC alone is unreactive at the set voltage, and that DEHP plays a role in the paired-electrolysis reaction. Without intending to be bound by theory, it is believed that the DEHP serves as a redox mediator.

The on/off reactivity was not observed under galvanostatic (constant-current) conditions because the working potentials of the electrode automatically adjusted to the large potentials needed to reduce PVC directly.

### Example 8: PVC Attenuates Reoxidation of DEHP Radical Anion

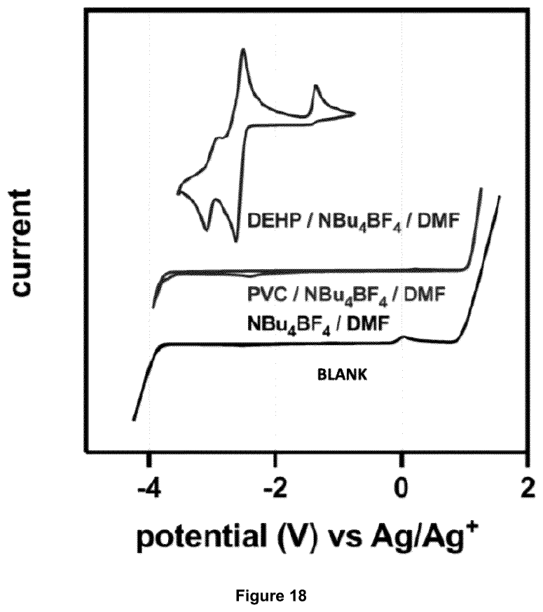

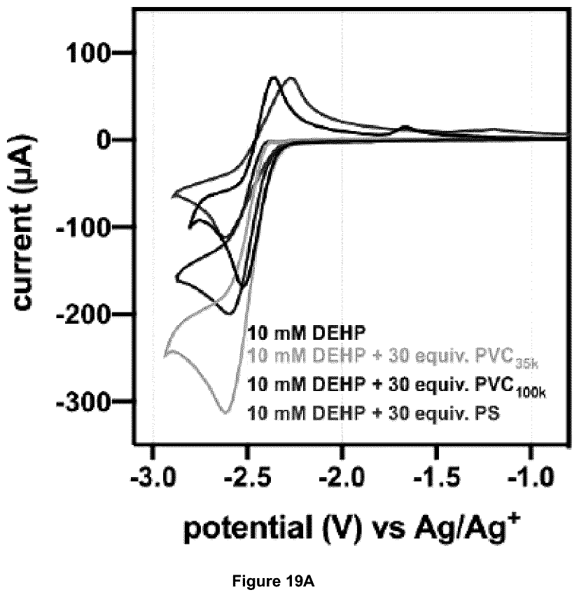

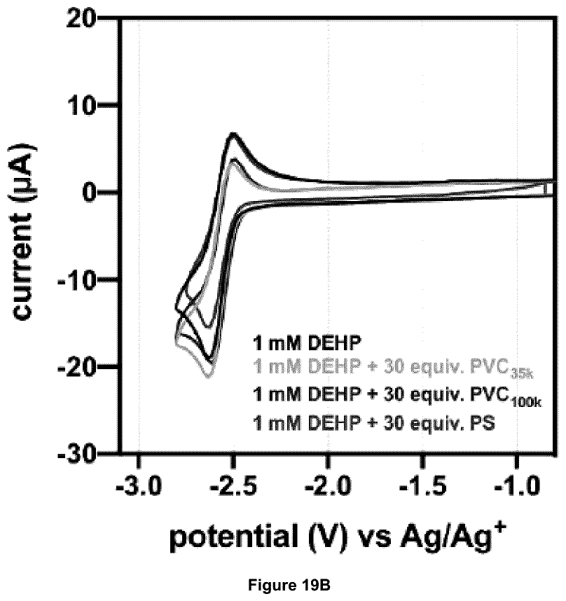

A cyclic voltammogram of PVC alone revealed that it does not exhibit any redox activity within the solvent window. By contrast, DEHP undergoes two successive reductions (E1/2=−2.56 V and −2.74 V versus Ag/Ag⁺) with a redox profile similar to one previously reported in acetonitrile (FIG. 18). FIG. 18 shows the CVs of a blank solution, 10 mM PVC35k calculated per repeat unit, and 10 mM DEHP. CVs were collected in 0.1 M NBu4BF4 in DMF using a 100 mV/s scan rate The first reduction is semi-reversible (ipa/ipc<1), whereas the second reduction is essentially irreversible (ipa/ipc=0), even at a fast scan rate (100 mV s⁻¹).

CVs of the first 1e⁻ redox couple of DEHP were collected in the presence of increasing equivalents of PVC. Equivalents were calculated by repeat unit mass and are effectively the same the number of C—Cl bonds. FIGS. 19A and 19B show CVs of DEHP at 10 mM (A) and 1 mM (B) and DEHP mixed with 30 equiv. PVC35k, PVC100k, or PS. CVs were collected in 0.1 M NBu4BF4 in DMF using 100 mV/s scan rate. CVs were initially collected using a 10 mM concentration of DEHP (FIG. 19A). The concentration was reduced by 10-fold (1 mM DEHP; FIG. 19B) to minimize viscosity/diffusivity effects upon addition of excess polymer. Although attenuated at lower net concentrations, PVC still affected the reversibility of the 1e⁻ redox couple observed for DEHP. Control studies using polystyrene (average MW: 35,000 g/mol, lot #MKCD6541) showed a negligible effect on the 1e⁻ redox couple observed for DEHP, suggesting changes in solution viscosity were not significant

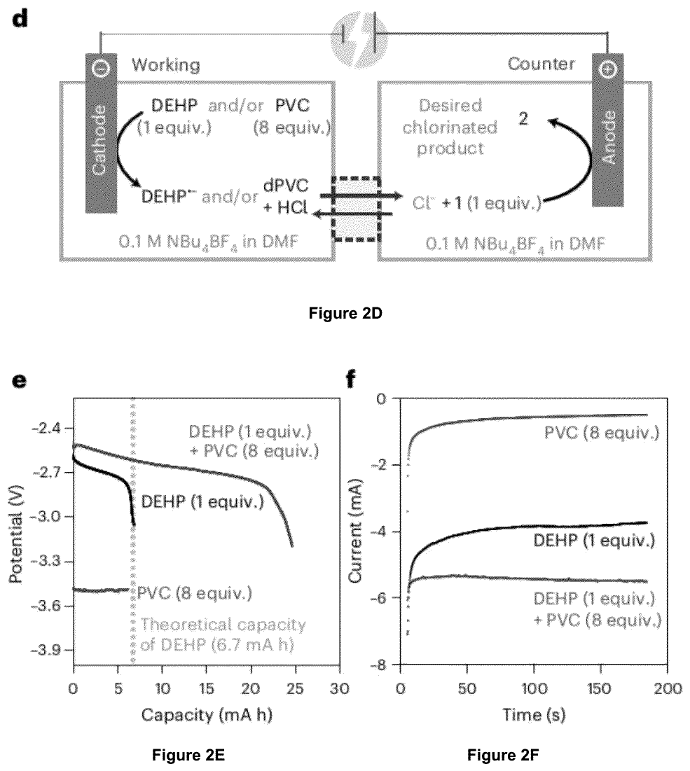

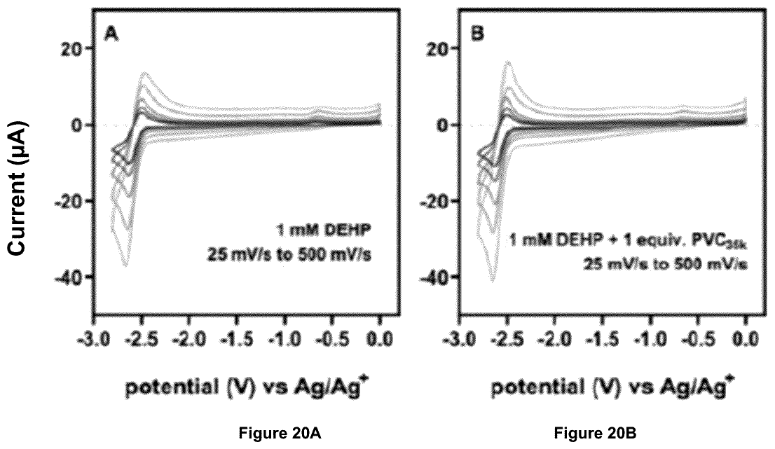

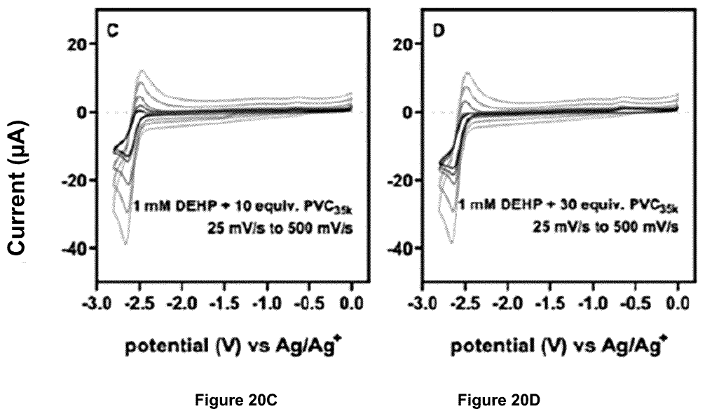

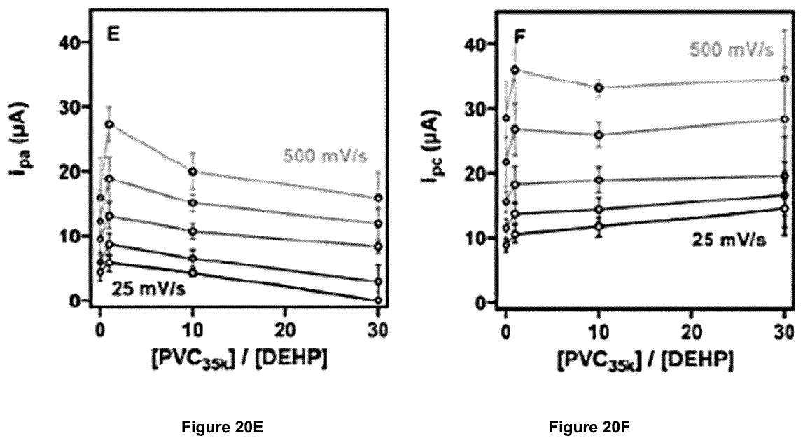

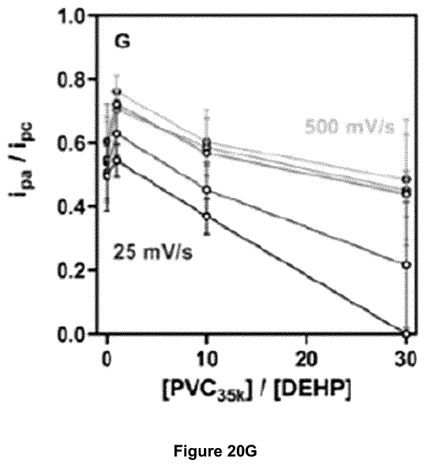

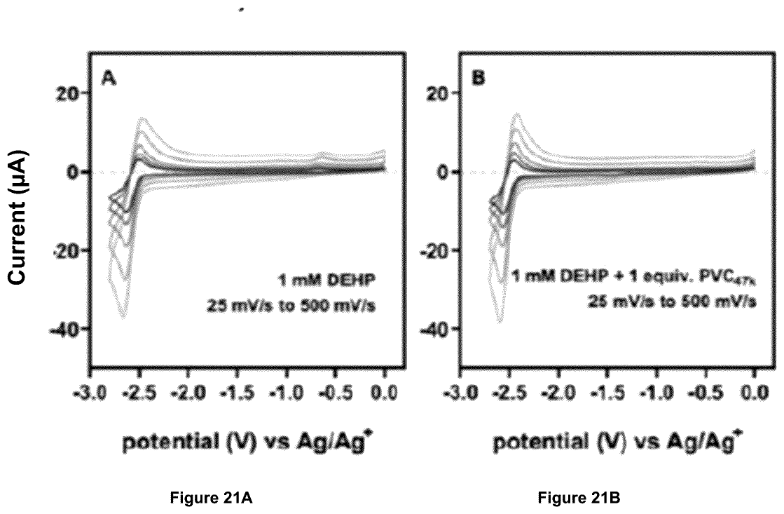

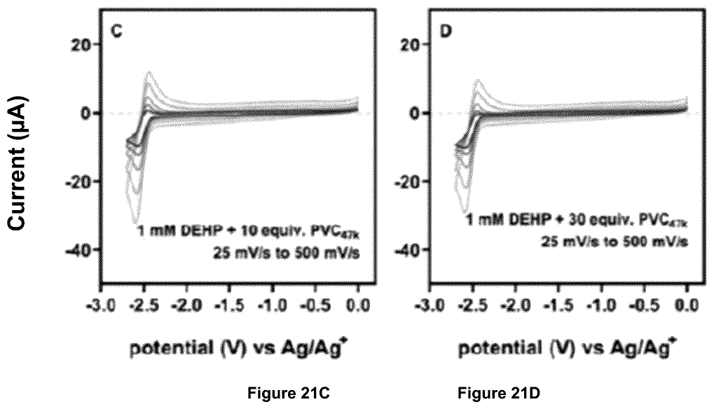

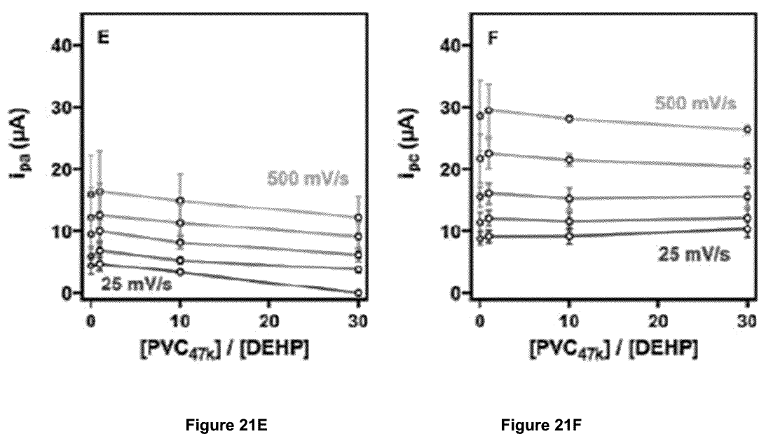

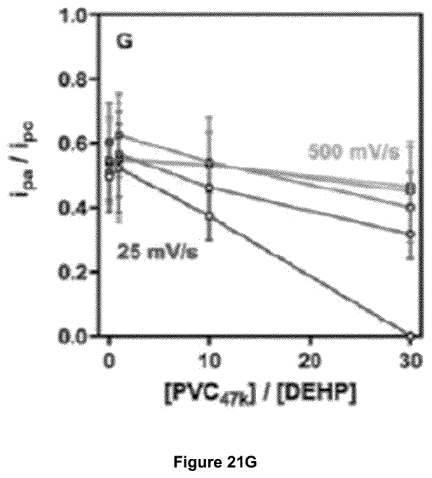

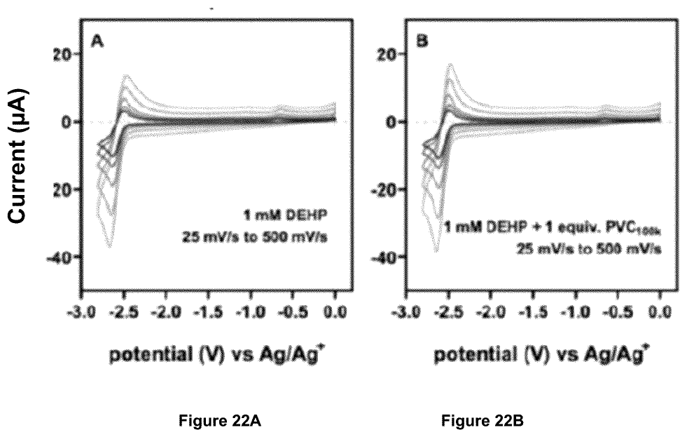

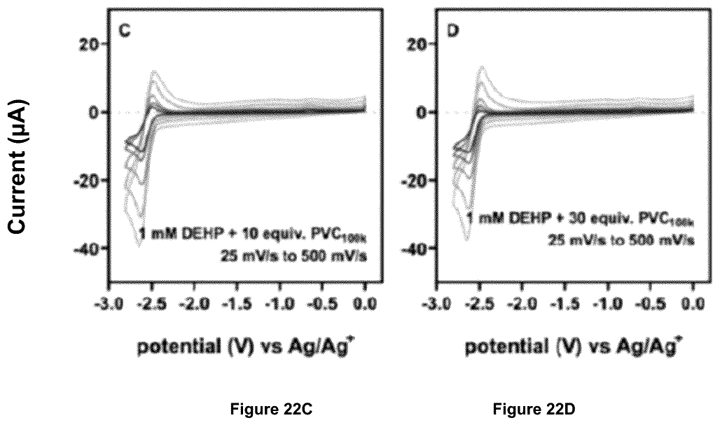

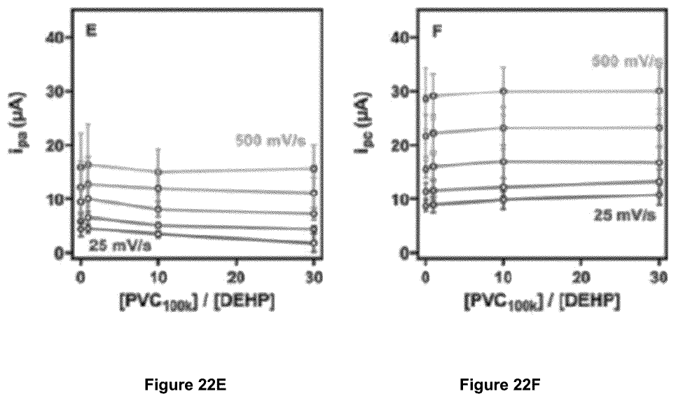

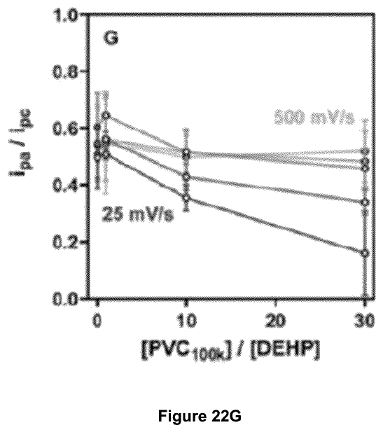

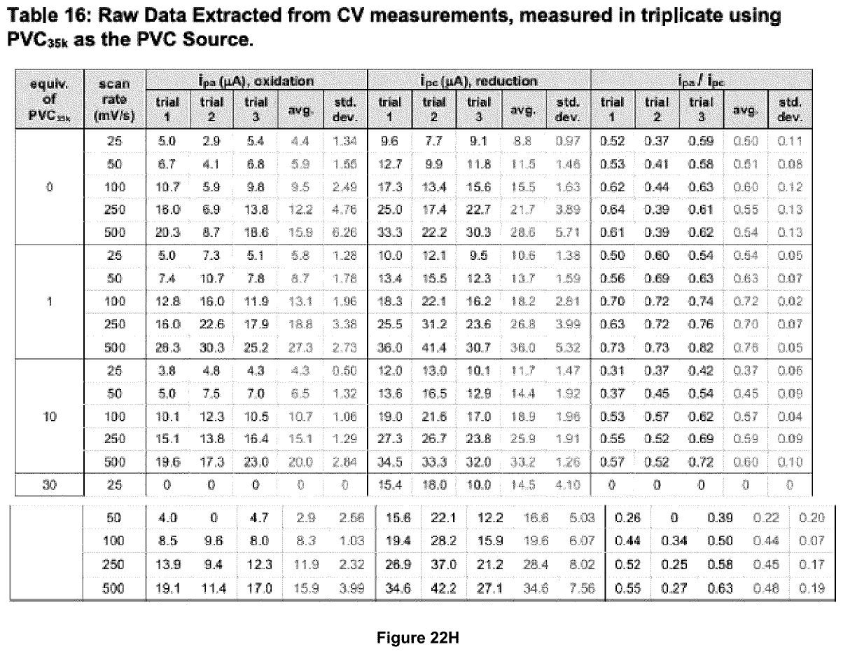

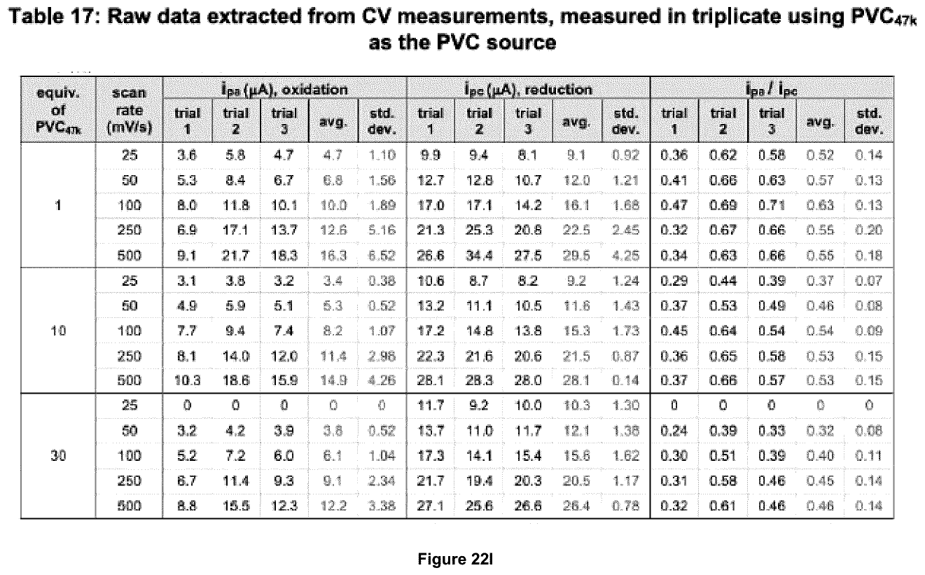

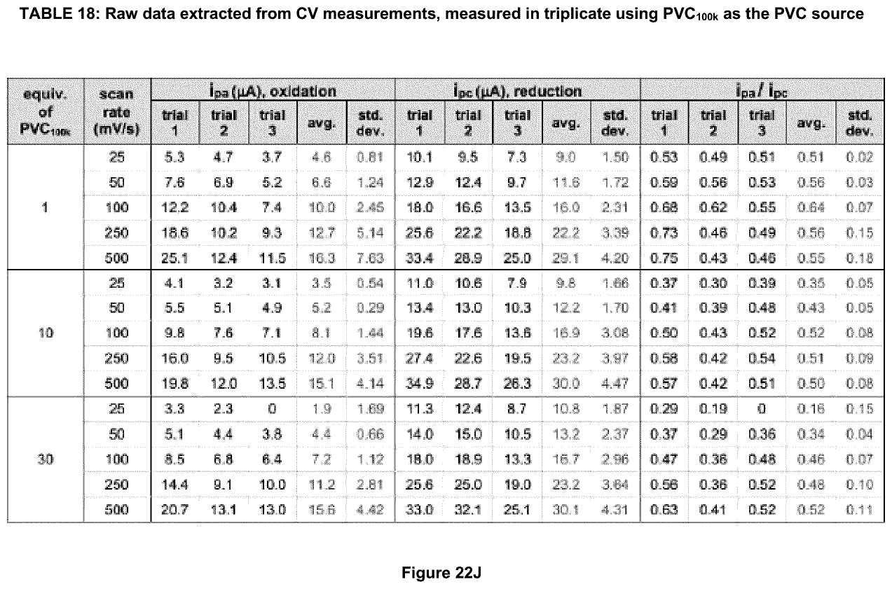

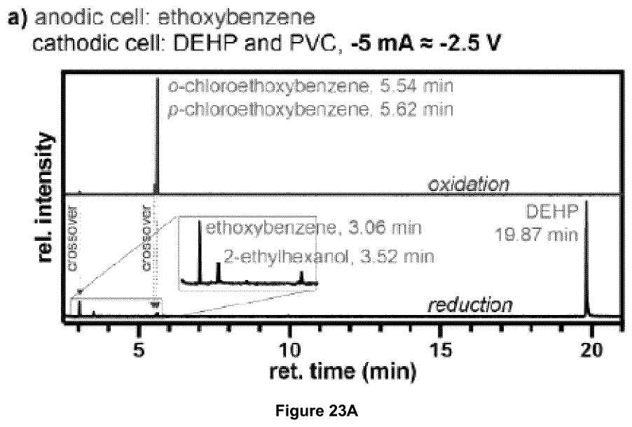

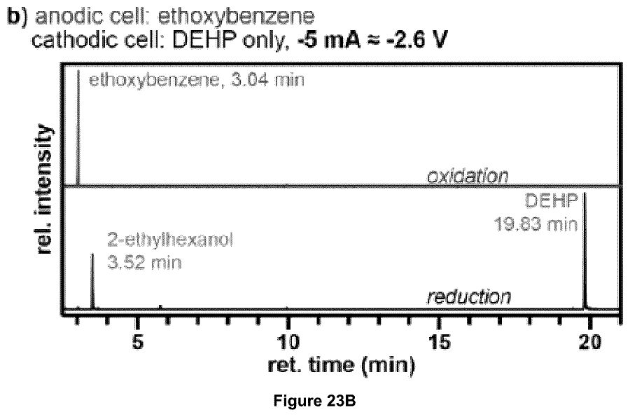

The reversibility of the first DEHP reduction decreases when PVC is added, which suggests electron transfer is occurring between the two species. Using a protocol similar to the mediated electrochemical reduction of butyl halides³⁷, increasingly higher concentrations of PVC₃₅ₖ, PVC₄₇ₖ or PVC₁₀₀ₖ ([PVC]=0-30 mM in repeat units) was added to a solution of DEHP (1 mM) and the reversibility of the first DEHP reduction (FIG. 2) was measured. The overall concentration of PVC was kept low to minimize the viscosity effects (see Table 15) and these data were measured in triplicate for multiple scan rates for each molar mass PVC (FIGS. 20-22, and Tables 16-18 in FIGS. 22E-22G).

TABLE 15
Raw data extracted from CV measurements of DEHP
mixed with 30 equiv. PVC35k, PVC100k, or PS.

| | 10 mM DEHP | | | 1 mM DEHP | | |
|---|---|---|---|---|---|---|
| polymer | ipa (μA) | ipc (μA) | | ipa (μA) | ipc (μA) | |
| added | oxidation | reduction | ipa/ipc | oxidation | reduction | ipa/ipc |
| none | 92.0 | 168.8 | 0.54 | 10.3 | 17.3 | 0.59 |
| PVC35k | — | 314.3 | — | 7.8 | 19.5 | 0.40 |
| (30 equiv.) | | | | | | |
| PVC100k | — | 199.1 | — | 7.9 | 18.0 | 0.44 |
| (30 equiv.) | | | | | | |
| PS | 71.4 | 112.5 | 0.63 | 9.2 | 13.8 | 0.67 |
| (30 equiv.) | | | | | | |

It was observed that the reversibility of the first DEHP reduction decreased with increasing concentration of PVC, which suggests that the singly reduced DEHP (DEHP·⁻) transfers an electron to the PVC (FIG. 2c) and, as a consequence, exhibits a lower return oxidation current. This trend was magnified at slower scan rates, wherein the DEHP·⁻ has more time to encounter PVC. Combined, these studies are consistent with DEHP serving as a redox mediator in the PVC reduction.

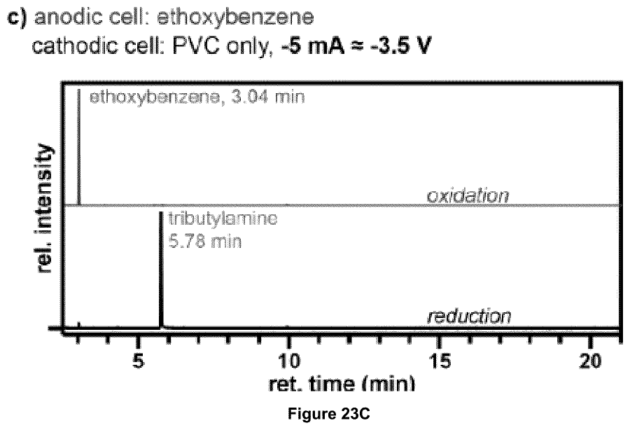

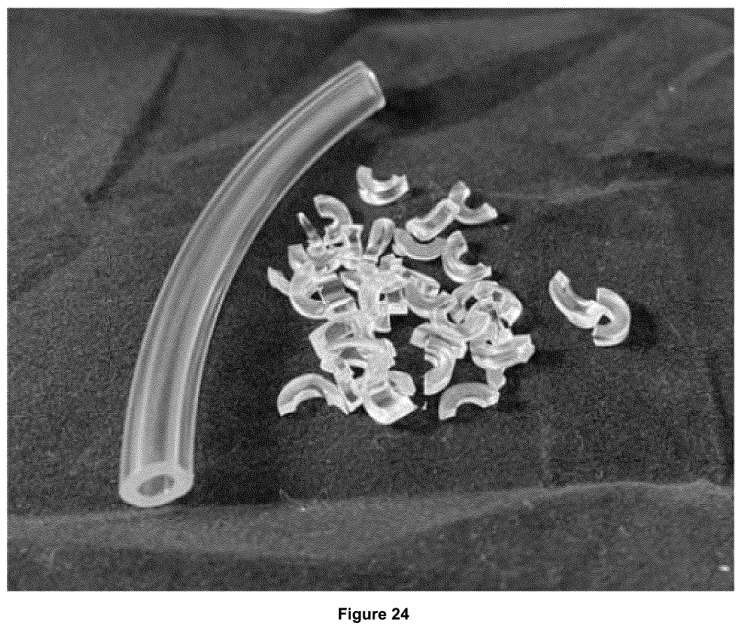

Bulk electrolysis shows plasticizer-mediated PVC reduction Further evidence of DEHP's role as a redox mediator was obtained using a preparative-scale set-up that mimicked the electrosynthesis conditions. More specifically, bulk electrolysis was performed in a divided cell that contained DEHP and/or PVC at the cathode/working electrode, and arene 1 at the anode/counter electrode (FIG. 2d). These experiments were used to determine the voltage required to generate a set current (galvanostatic conditions; FIG. 2e) and vice versa (potentiostatic conditions; FIG. 2f). Under constant-current conditions, DEHP alone generated −2.6 V of overpotential until reaching its theoretical capacity (6.7 mA h). Gas chromatography-mass spectrometry (GC-MS) analysis after electrolysis showed that DEHP was partially degraded into 2-ethylhexanol and presumably other undetectable fragments, and that arene 1 remained unreacted at the anode (FIG. 23). When PVC₄₇ₖ (8 equiv.) was added to DEHP (1 equiv.) and electrolyzed under the same current, a slightly smaller overpotential (−2.5 V) was observed and the capacity was substantially increased (24 mA h), indicating that the mixed system generated more electrons than DEHP alone. Post-reaction analysis revealed less DEHP degradation than without PVC, and quantitative conversion of arene 1 to chlorinated arene 2. By contrast, bulk electrolysis on PVC alone required a much larger voltage (−3.5 V) to reach the same current. This voltage matches the solvent potential window, where solvent and/or electrolyte degradation probably occurs. Indeed, GC-MS analysis showed that tributylamine was formed at the cathode (from electrolyte degradation) and arene 1 remained unreacted at the anode. The inability to chlorinate arene 1 with PVC alone in the bulk electrolysis set-up differs from the galvanostatic synthetic trials, which proceeded with or without DEHP, albeit to different yields. It is possible that the exclusion of O2 and H2O in the bulk electrolysis set-up, but not the synthetic trials, may have affected PVC degradation and arene chlorination. Bulk electrolysis experiments were also performed under constant-voltage to simulate potentiostatic reaction conditions (FIG. 2f). Each electrolysis was conducted for several minutes until a steady-state current was reached. When the cathode was set to −2.5 V, DEHP alone generated −3.8 mA of reduction current. Meanwhile, PVC alone produced a much smaller reduction current (−0.5 mA) at this voltage, indicating essentially no electrochemical activity. The largest reduction current (−5.5 mA) was generated when DEHP was mixed with PVC, indicating that more electrons were flowing through the system. These data again support that the PVC—when restricted by a voltage cutoff—can only be reduced when DEHP is present.

### Example 9: Mixed Plastic Waste

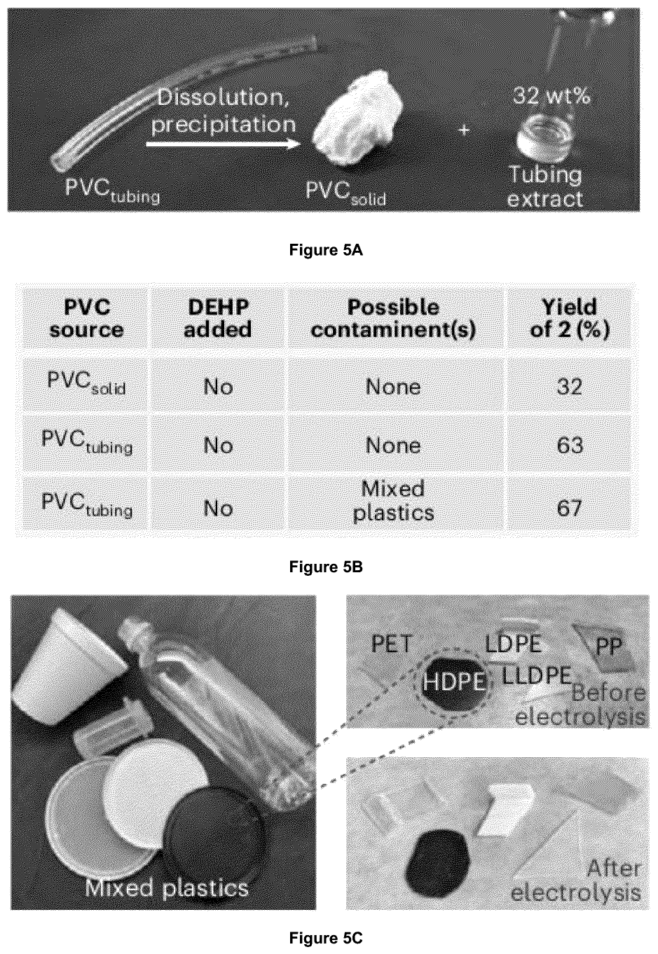

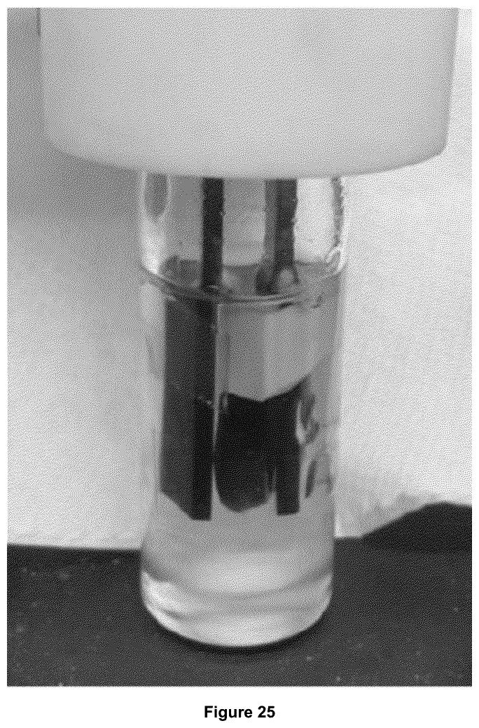

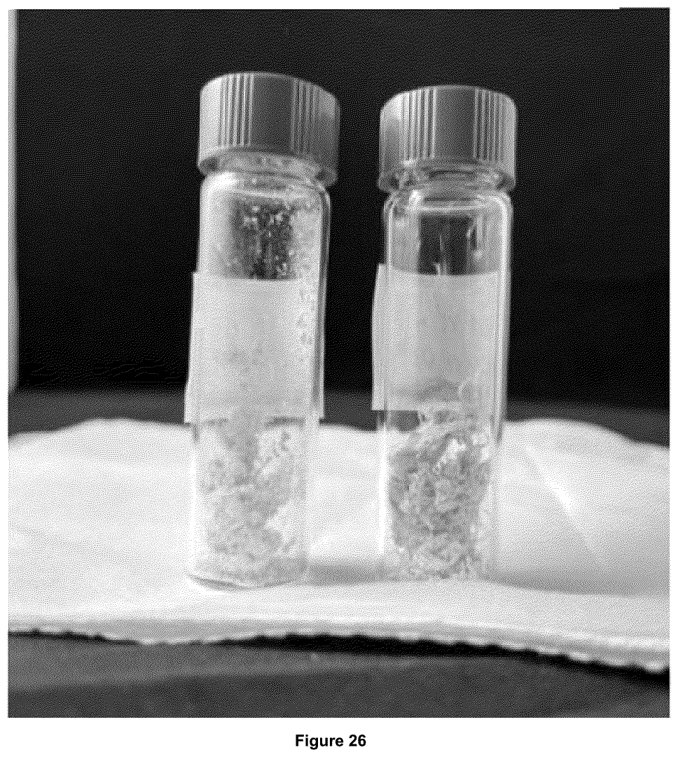

To simulate a mixed-plastic-waste stream, the reaction efficiency was evaluated with other plastic waste added (FIG. 5C). This reaction was performed according to General Procedure C, using 0.30 g of PVCtubing (˜67% by weight PVC=˜0.20 g PVCsolid). Other plastics were collected from household items (water bottle, food container lids, medicine container, Styrofoam cup) and the polymer type was identified by the recycling code information listed on the item. Each plastic item cleaned with water, wiped dry, and cut into a small piece that was added into the reaction mixture (FIG. 25). The reaction was performed once. The masses of each plastic piece were: 63 mg PET, 84 mg HDPE, 96 mg LDPE, 56 mg LLDPE, 80 mg PP, and 58 mg PS. The overall yield of Compound 2 was unchanged when similar masses of PET, HDPE, LDPE, LLDPE, PP and PS were added to the reaction mixture. Moreover, the plastic pieces made from PET, HDPE, LDPE, LLDPE and PP were insoluble in DMF and could be easily filtered out after the reaction was complete. These reactivity and solubility differences enable a facile separation of PVC from most other waste plastics. Table 19 provides a summary of the data.

TABLE 19
Summary of data collected from reactions using real plastic items.

| | | possible | conversion | consumption | yield | |
|---|---|---|---|---|---|---|
| Entry | PVC source | contaminant(s) | of 1 (%) | of [symbol] | of 2 (%) | NBu3 |
| 1 | PVCsolid | none | 66 | — | 30 | y |
| 2 | PVCsolid | none | 55 | — | 33 | y |
| 3 | PVCtubing | none | 85 | 95 | 62 | n |
| 4 | PVCtubing | none | 89 | 91 | 64 | n |
| 5 | PVCtubing | mixed plastics | 76 | 100 | 67 | n |

[symbol]

indicates data missing or illegible when filed

### Example 10: Polymer Characterization after Electrolysis

After electrolysis according to General Procedure C, dPVC was recovered. To recover the dPVC, the reaction mixture was poured into 200 mL methanol while stirring to precipitate the polymer. The polymer was collected by filtration and then redissolved in 5 mL THF. The solution was poured into 200 mL methanol while stirring to precipitate the polymer, again. The polymer was collected by filtration, and the dissolution and precipitation were repeated one more time. The polymer was collected by filtration and dried under vacuum overnight. FIG. 26 is a photograph of the dPVC recovered from the reactions performed using PVC₄₇ₖ with 1 equiv. of DEHP (left) and with no DEHP (right).

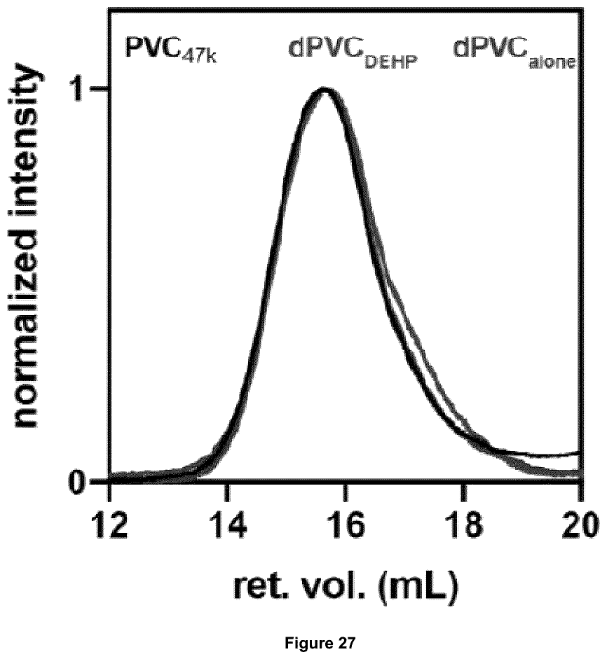

The resulting polymer (dechlorinated PVC, dPVC) was characterized to determine the extent of dechlorination and any new structural features. Briefly, the soluble polymer recovered from the standard galvanostatic reaction listed in FIG. 1—performed with DEHP (dPVC_{DEHP}) and without DEHP (dPVCₐₗₒₙₑ)—was isolated and characterized. Neither dPVC sample showed a substantial change in Mn relative to the unreacted PVC via size-exclusion chromatography (FIG. 27), which is unsurprising given the large excess of PVC used and expected ˜20% conversion of functional groups within each chain. Elemental analyses performed on the soluble residue indicated that both dPVC samples had reduced Cl content (dPVC_{DEHP}, 44% Cl; dPVCₐₗₒₙₑ, 53% Cl) relative to unreacted PVC (57% Cl). Elemental analysis indicated that both dPVC samples contained chlorinated repeat units, but dPVC recovered from reactions that include DEHP show a higher degree of dechlorination. An estimate of % dechlorination was calculated using the equation below.

$$\% dechlorination = ( 1 - \frac{\% Cl dPVC}{\% Cl PVC} ) * 100 \%$$

TABLE 20
Summary of elemental analysis data collected on PVC
and dPVC recovered from reactions performed using
PVC47k with 1 equiv. of DEHP and with no DEHP.

| | mass % | | | |
|---|---|---|---|---|
| | PVC | PVC | | dPVC, |
| element | (theoretical) | (experimental) | dPVC, DEHP | no DEHP |
| C | 38.44 | 38.7 | 46.0 | 39.0 |
| H | 4.84 | 5.0 | 5.8 | 4.9 |
| N | 0.00 | 0.0 | 0.0 | 0.0 |
| Cl | 56.72 | 56.5 | 43.5 | 53.4 |
| total | 100 | 100.2 | 95.3 | 97.3 |

| | | |
|---|---|---|
| approx. % dechlorination | 23.0% | 5.5% |

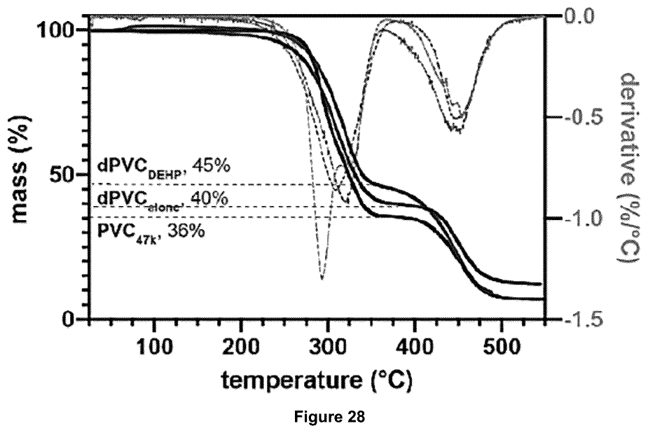

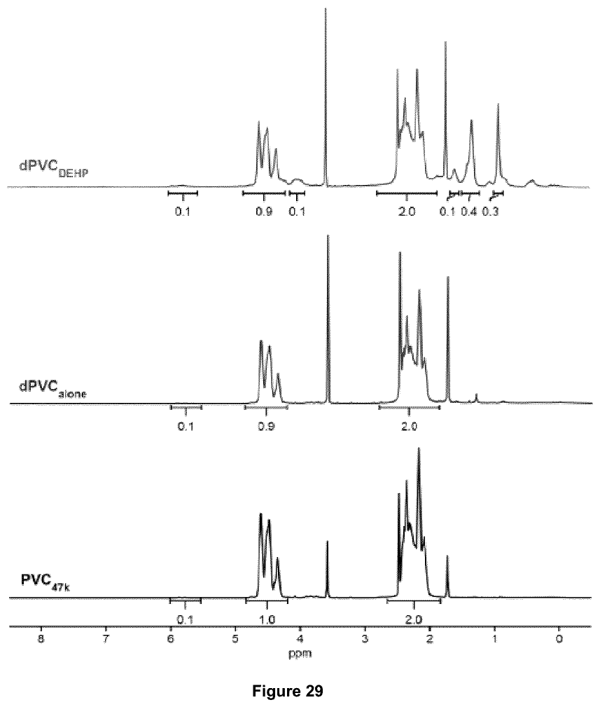

Similarly, less mass (HCl) was lost at lower temperatures in the thermal gravimetric analysis for both dPVC samples compared with unreacted PVC, consistent with dechlorination (FIG. 28). TGA was used to estimate the relative amount of chlorine-containing repeat units in each dPVC sample. The thermal mass loss profile of PVC shows two major steps. The first step occurs at ˜300° C. and generates mostly HCl along with some hydrocarbon degradation products.² The second step occurs at ˜450° C. and degraded the remaining hydrocarbon content of the material. PVC₄₇ₖ retained 36% of its mass after the first thermal step (64% mass loss). Samples of dPVC are expected to show less mass loss during the first stage because some HCl has already been removed from the polymer. This change in mass is observed. Recovered dPVC from reactions performed with DEHP retains 45% mass (55% mass loss), while dPVC recovered from reaction without DEHP retains 40% mass (60% mass loss). In both cases, less mass is lost from dPVC, further supporting that some HCl was emitted during electrolysis. This data suggests that more HCl was lost when DEHP was included in the reaction, which agrees with the higher chlorination yields observed under these conditions. FIG. 29 shows stacked ¹H NMR spectra of dPVC recovered from reactions with and without DEHP.

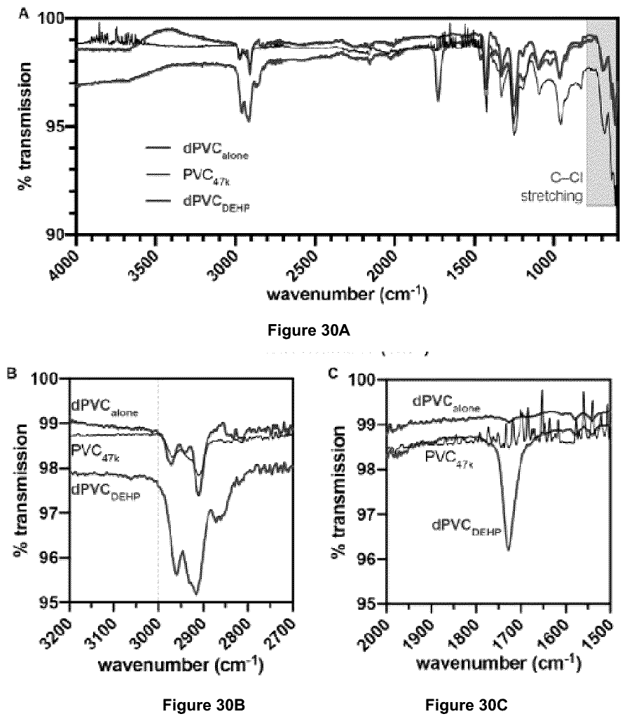

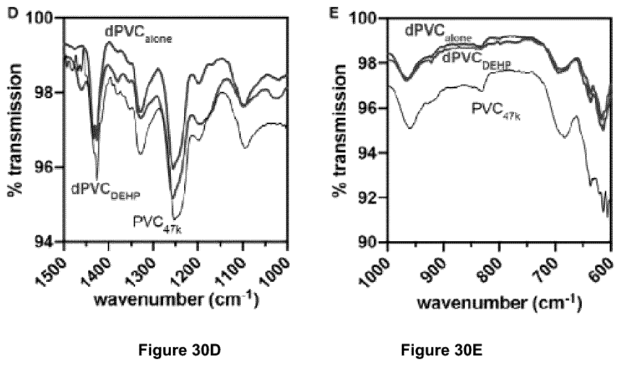

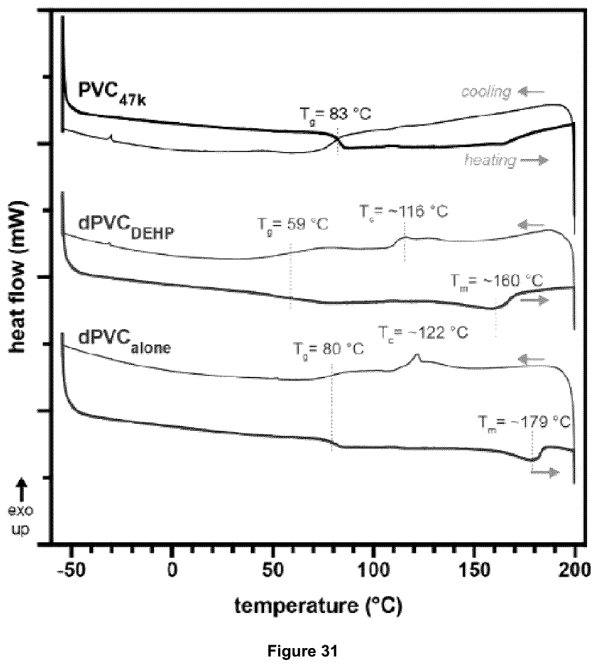

Differential scanning calorimetry (DSC) data showed a lower glass-transition temperature for dPVC_{DEHP} (Tg=59° C.) relative to unreacted PVC (Tg=83° C.), suggesting internal plasticization, potentially due to the 2-ethylhexyl side-chains (FIG. 31). Indeed, both 1H NMR and infrared spectra of dPVC_{DEHP} showed evidence of 2-ethylhexyl substitution (FIG. 30). Referring to FIG. 30, the IR spectrum of dPVC recovered from reactions performed with DEHP shows weak Csp3-H stretching from 2850-2900 cm⁻¹ and a strong carbonyl stretch at 1726 cm⁻¹, which may be from oxidation of the polymer backbone or from residual DEHP. Otherwise, the IR spectra of both dPVC samples do not vary qualitatively from PVC starting material, suggesting the polymer contains a significant portion of unreacted PVC repeat units. One consequence is that these substituents will increase the carbon content and lead to an overestimation of the dechlorination in the elemental analysis. Both dPVC samples showed a much higher degree of crystallinity relative to unreacted PVC (FIG. 31).

To further elucidate the polymer structure, a tenfold excess of dimethylphthalate (DMP) was used in the electrolysis reaction to generate dPVC_{DMP}.

The electrolysis reaction using the following conditions using an undivided cell. PVC (200 mg, 3.2 mmol (repeat unit), 8.1 equiv. (repeat unit)), NBu4BF4 (264 mg, 0.80 mmol), dimethylphthalate (0.653 mL, 4.0 mmol, 10.0 equiv.), ethoxybenzene (0.510 mL, 0.4 mmol), tridecane internal standard (0.500 mL), and DMF (8 mL) were added to a 10 mL ElectraSyn® reaction vessel equipped with Teflon coated magnetic stir bar (egg-shaped, 16 mm length) and two graphite electrodes (0.8×0.2×5.20 cm). All reagents were dissolved by stirring for at least 15 min, then the reaction mixture was subjected to constant current electrolysis (30 mA) with alternating polarity (15 min) at rt. The reaction was stirred at 400 rpm for 24 h.

After electrolysis, dPVC was recovered using the following protocol. The reaction mixture was poured into 200 mL methanol while stirring to precipitate the polymer. The polymer was collected by filtration and then redissolved in 5 mL THF. The solution was poured into 200 mL methanol while stirring to precipitate the polymer, again. The polymer was collected by filtration, and the dissolution and precipitation processes were repeated one more time. The polymer was collected by filtration and dried under vacuum overnight.

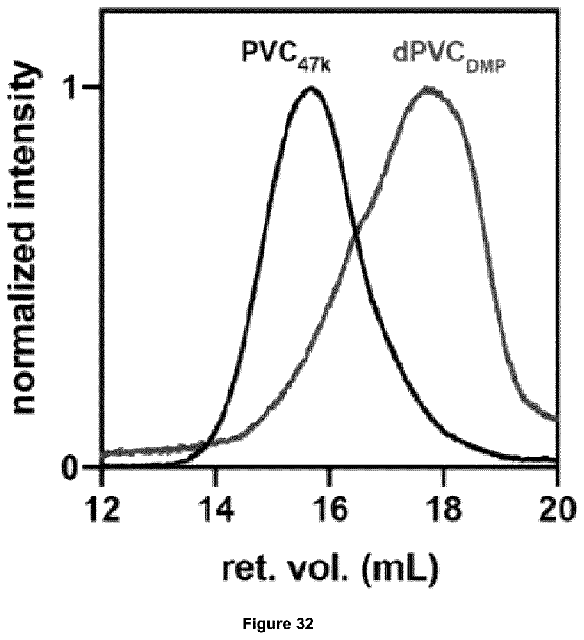

The increased phthalate loading was intended to increase the signal intensity of substituted products and the methyl ester side-chain was intended to simplify the aliphatic region of the 1H NMR spectrum. A substantial decrease in molar mass was observed via size-exclusion chromatography (Mn=14 kg mol-1; FIG. 32) compared with the unreacted PVC47k, suggesting some chain scission was occurring. The 1H NMR spectrum reveals a complex mixture of substituents, including alkenyl, aromatic, ester/ether and alkyl groups, alongside unreacted PVC units (FIG. 3).

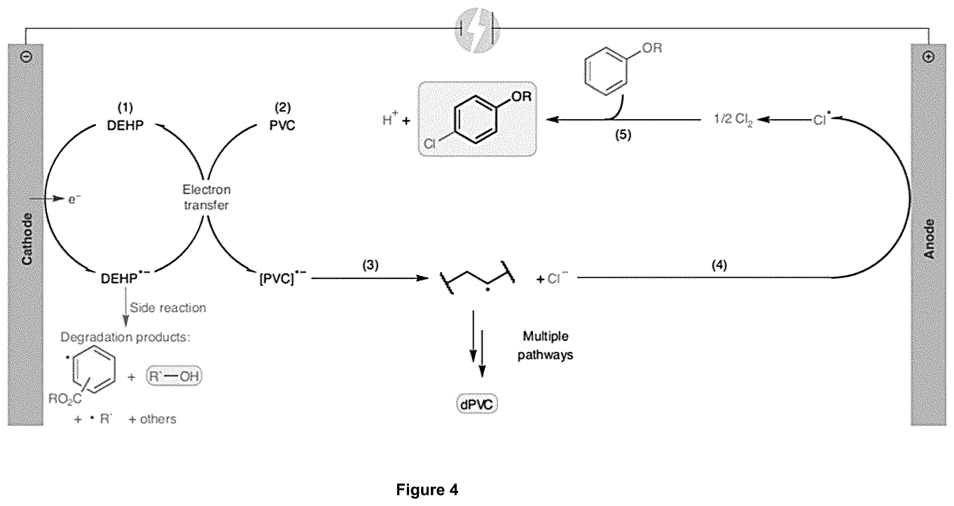

With DEHP present, an indirect reduction of PVC occurs wherein DEHP transfers the electron between the cathode and PVC (FIG. 4). Without DEHP present, PVC reduction requires a much larger voltage, and may react through direct interaction at the electrode, or with the solvent/electrolyte degradation products that were observed at these extreme voltages (FIG. 23). Ultimately, the milder and more controlled conditions offered by the galvanostatic DEHP-mediated pathway are preferred. Furthermore, this pathway effectively repurposes the plasticizer in plastic waste by using it as a redox mediator.

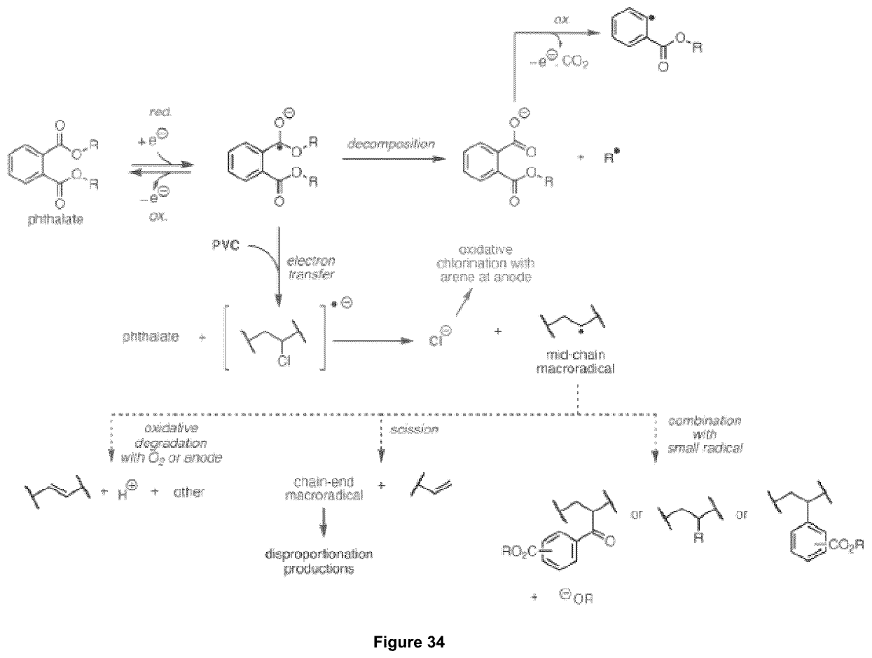

Regardless of how the electron is added to PVC, upon reduction, the chloride anion (Cl−) is believed to be cleaved from the PVC via a concerted electron-transfer-bond-breaking process, as has been suggested for small-molecule alkyl halides. The resulting carbon-based macroradical should be highly reactive and probably undergoes several reaction pathways (FIG. 34). Based on NMR spectral data, the PVC macroradical reacts with alkyl and aryl radicals generated from the concomitant and unavoidable DEHP degradation. The PVC macroradical may also undergo scission reactions to form lowered-molar-mass polymers, which in turn generate alkenes and end-group macroradicals that probably disproportionate to generate alkene and alkane end-groups. Poly(vinyl chloride)-based macroradicals may also react with O₂ or H₂O, and further facilitate dehydrochlorination, as has been observed in other systems. Depending on the location, the formed alkenes may generate allylic C—Cl bonds, which would be even more susceptible to reductive bond cleavage. The observed acidification of the reaction medium suggests that H+ is also released from the polymer upon reduction, perhaps through a concerted deprotonation oxidation (FIG. 32). Notably, the net release of HCl from PVC is a common reaction pathway with nucleophiles, heat, base or acid.

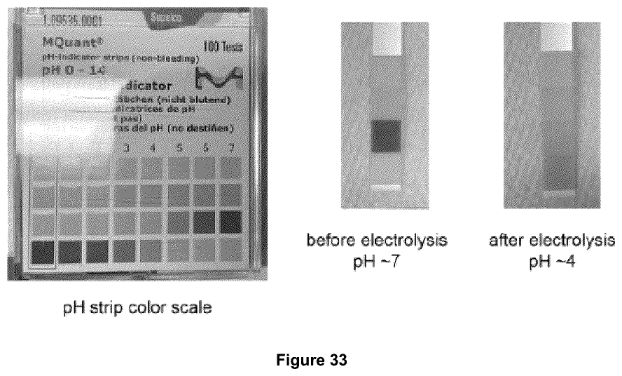

The pH of the electrolysis reaction was monitored using pH strips (FIG. 33). The electrolysis was performed following General Procedure C using PVC47k and DEHP. The arene substrate was not included in the reaction, to remove effects of the arene chlorination on pH. The pH strips indicated that the reaction became more acidic during electrolysis. The change in pH is attributed to the degradation of PVC during electrolysis, likely releasing HCl.

Control reactions were performed to identify the possible chlorine species in solution. The chlorination reaction did not proceed when PVC was not included in the reaction (entry 2), supporting the role of PVC as the chloride source. The reaction proceeded cleanly when HCl was used as the chloride source (entry 3), supporting the notion that

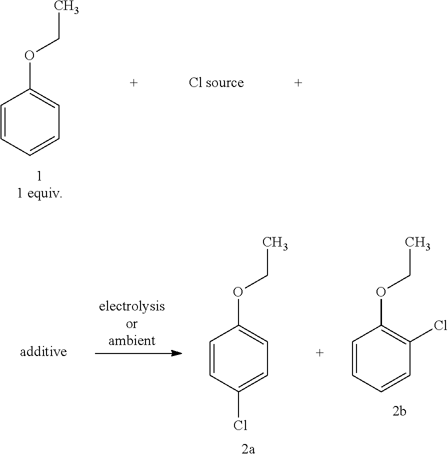

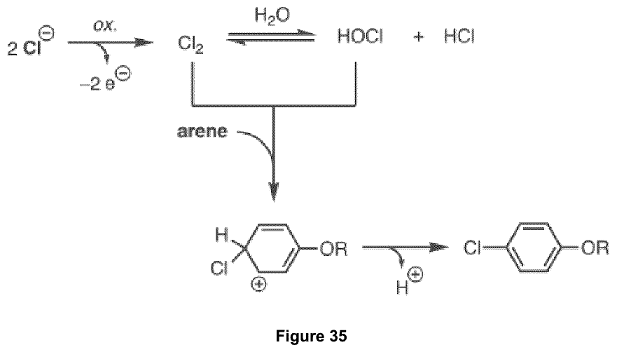

chloride ions are likely generated from PVC reduction. When TEMPO was added, no product was observed in the PVC/DEHP reaction (entry 4) nor the HCl control reaction (entry 5), suggesting that radical intermediates may be involved in the oxidative chlorination half-reaction, either to convert Cl⁻ into an active chloride species or in the arene chlorination step. The reaction proceeded with both NaOCl (entry 6) or Cl2 (entry 7) as the chloride source without any electrical potential, suggesting that either hypochlorite or chlorine could be the active chloride species in this reaction. The fact that the PVC/DEHP reaction proceeds better open to air suggests that water may be generating hypochlorite (OCl⁻) in situ. Taken together, this data supports the possible reaction pathway in which PVC is reductively degraded into HCl, and then Cl⁻ is converted into Cl2 (or OCl⁻), which reacts with arene 1 to generate product 2. FIG. 35 shows a proposed mechanism for oxidative arene chlorination.

TABLE 21
Reactions performed to vary the possible chloride source.

| | | | | yield of |
|---|---|---|---|---|
| entry | Cl source | additive | conditions | 2 (%) |
| 1 | PVC47k (8 equiv.) | DEHP (1 equiv.) | electrolysis^{a} | 85% |
| 2 | none | DEHP (1 equiv.) | electrolysis^{a} | 0% |
| 3 | HCl (3.2 equiv.)^{b} | none | electrolysis^{a} | quant. |
| 4 | PVC47k (8 equiv.) | DEHP (1 equiv.), | electrolysis^{a} | 0% |
| | | TEMPO | | |
| | | (3.5 equiv.)c | | |
| 5 | HCl (3.2 equiv.)^{b} | TEMPO | electrolysis^{a} | 0% |
| | | (3.5 equiv.)^{c} | | |
| 6 | NaOCl (3.2 equiv.) | none | ambient^{d} | 33% |
| 7 | Cl2 (excess) | none | ambient^{e} | quant. |

^{a}Electrolysis conditions: performed according to General Procedure C.
^{b}Concentrated HCl (37% w/w, 0.100 mL, 1.3 mmol) was used in place of PVC and DEHP and added to the reaction after dilution with DMF.
^{c}(2,2,6,6-Tetramethylpiperidin-1-yl)oxyl (TEMPO, 0.219 g, 1.4 mmol) was added to the to the reaction before electrolysis.
^{d}Ethoxybenzene (0.051 mL, 0.4 mmol) and tridecane (0.050 mL) were dispensed in a 20 mL vial and dissolved in DMF (6 mL). NaOCl solution (2.21 mL, 4.00-4.99% active chlorine) was added and the reaction stirred at rt for 16 h.
^{e}Ethoxybenzene (0.051 mL, 0.4 mmol) and tridecane (0.050 mL) were dispensed in a 25 mL round bottom flask and dissolved in DMF (8 mL). Cl2 was slowly bubbled into the solution and stirred at rt for 5 min. Cl2 was produced by slowly adding concentrated HCl (2 mL) to a 12.5% NaOCl solution (20 mL).

The released Cl⁻ ions ultimately chlorinate arene substrates at the anode. Oxidation of Cl⁻ to either chlorine (Cl₂) or hypochlorite (⁻OCl) intermediates have been proposed as the active chlorine species in electrochemical chlorination. Under the open-to-air reaction conditions, any Cl₂ formed may react with adventitious water to form ⁻OCl. Control reactions wherein Cl₂ or NaOCl was used as a chlorine source both proceeded without applied potential, further supporting that either could be the active chlorine species (Table 21). When TEMPO was added, no product was formed in the PVC/DEHP reaction nor the HCl control reaction, suggesting that radical intermediates may be involved in the oxidative chlorination, either to convert Cl− into a Cl radical or in the arene chlorination (Table 21)

### Example 11: Organic Compounds to be Halogenated

Chlorinated arenes are found in pharmaceuticals, agrochemicals, disinfectants, dyes, electrical goods and solvents. To determine the scope of this method and probe possible interference from either PVC and/or DEHP, various aromatic and heteroaromatic substrates were (FIG. 6). In an undivided cell, some substrates were consumed during electrolysis but did not yield any chlorinated products with PVC/DEHP, but did when HCl was used (for example, anilide 3). These studies suggest that some substrates may react directly with PVC and/or DEHP under the reductive conditions. To avoid this unintended reactivity, all subsequent reactions were performed in a divided cell in which the working and counter compartments were separated by an anion-exchange membrane. Compound classes that have been employed in other oxidative chlorination reactions (for example, substituted benzenes, thiophenes and several classes of N-heterocycles) were successfully employed herein using PVC as a chloride source. Some compounds, including indole derivatives, generated lower yields because other oxidative reaction pathways (dimerization/oligomerization) competed with chlorination. Chlorinated pharmaceuticals 13 and 14 could be directly formed under these conditions from their non-halogenated precursors.

### Example 12: Commercial PVC Tubing as Chloride Source

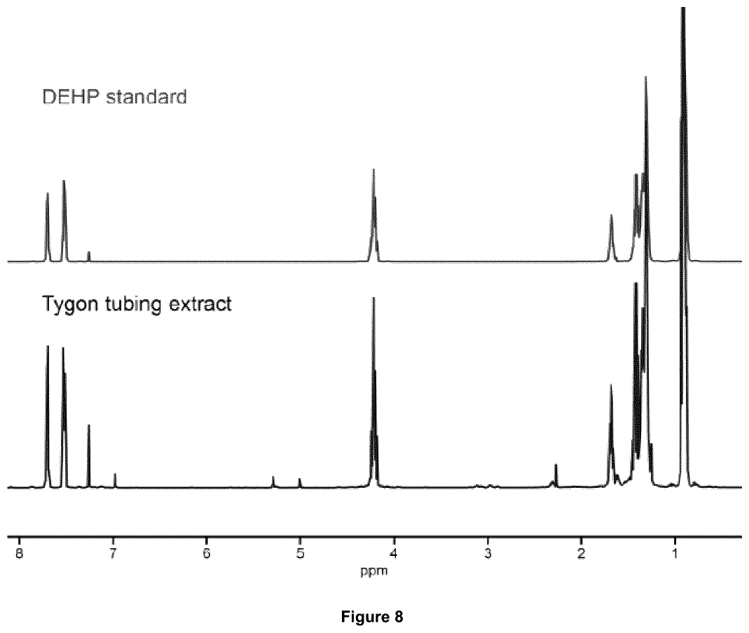

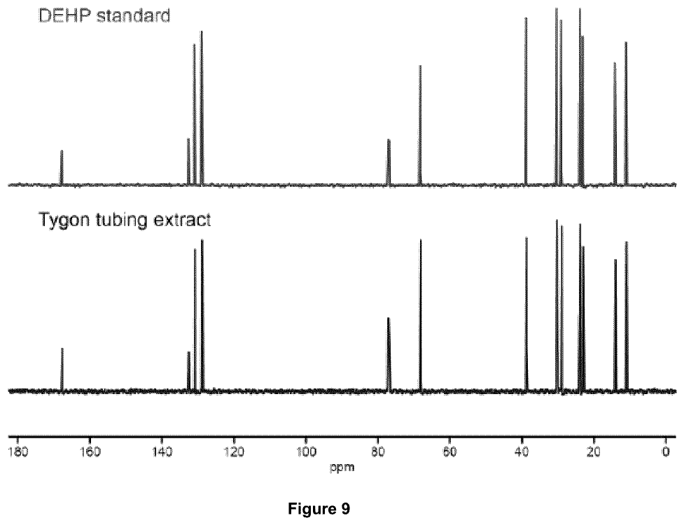

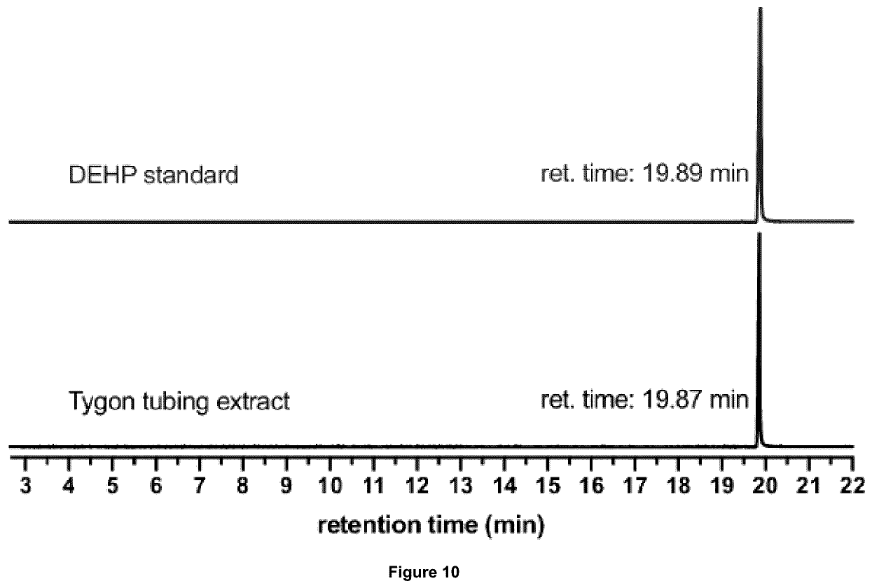

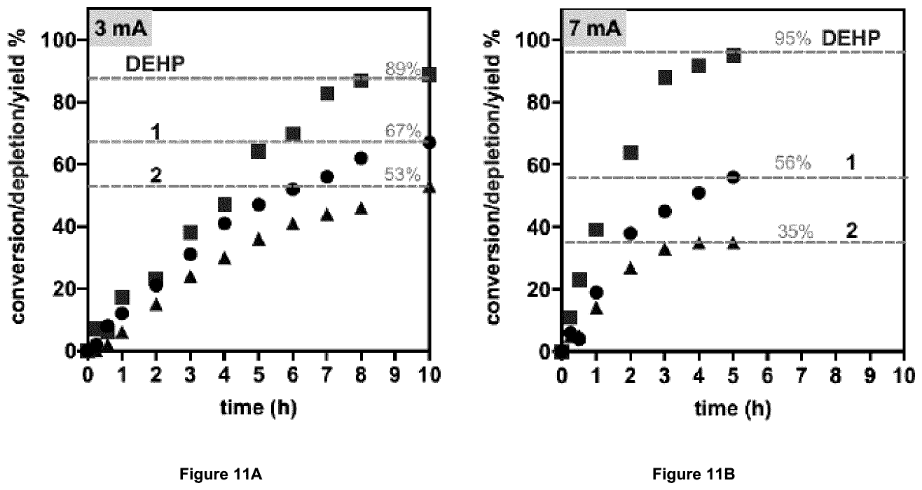

The method of the disclosure was performed using PVC tubing as the waste source. PVC tubing is commonly used for liquid and gas transport in the research, medical, and food and beverage industries (FIG. 5a). The PVC tubing contained 32% liquid additives by mass, determined to be DEHP by ¹H and ¹³C NMR spectroscopic analysis (FIGS. 8-10).

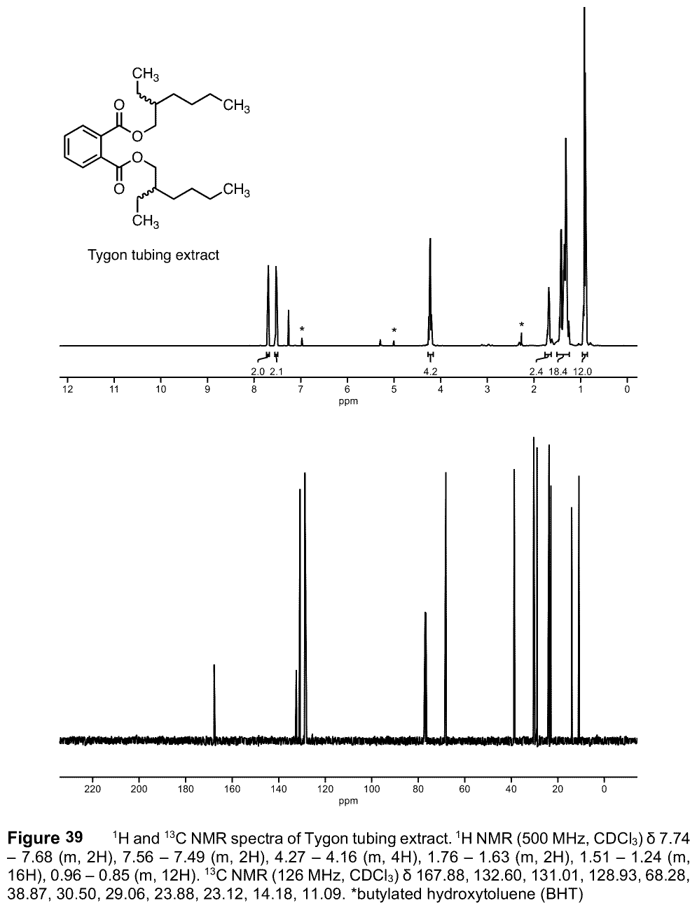

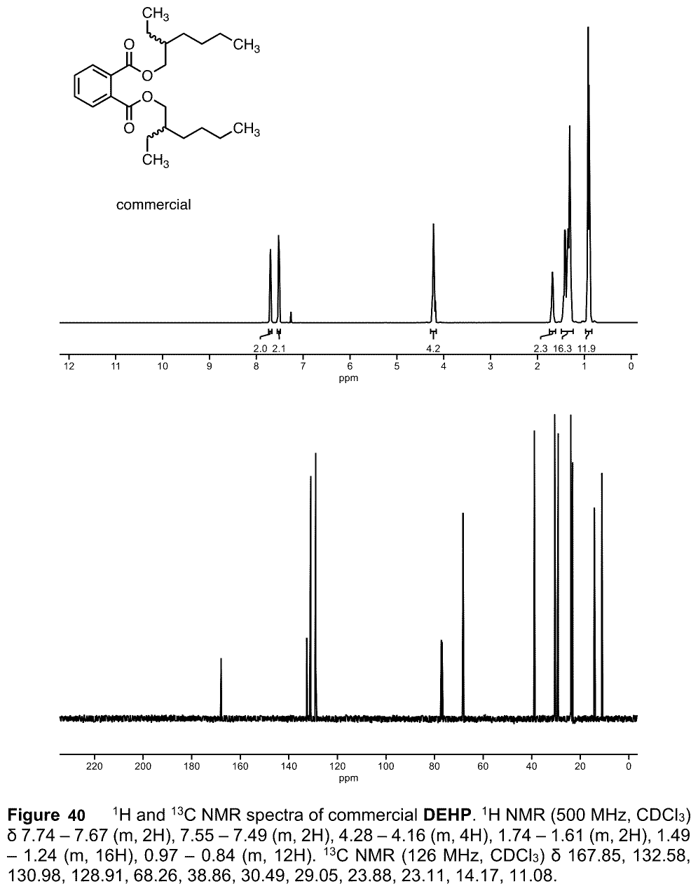

Extraction of plasticizer from Tygon tubing. A strip of Tygon tubing (4.145 g) was cut into small pieces (˜0.5 cm length) and stirred in THF (˜50 mL) at rt until fully dissolved (˜30 min). The polymer was precipitated by pouring into cold methanol (˜200 mL) while stirring. The polymer was collected by filtration, and the filtrate was concentrated under reduced pressure to give PVCsolid. This dissolution and precipitation process was repeated on the filtered polymer 2 more times. The combined filtrates were concentrated under reduced pressured and further dried under vacuum overnight to remove residual solvent. A colorless oil was obtained (1.308 g, 32% mass rel. to tubing input, 1:14 mol ratio DEHP:PVC repeat unit). Referring to FIGS. 39 and 40, NMR signals and GC retention times of extract matched commercial DEHP standard. Note, the commercial DEHP standard purchased from Sigma Aldrich is labeled “di(octyl) phthalate” by the supplier, but it is di(2-ethylhexyl) phthalate (FIGS. 8-10).

The reaction with the PVC_{solid} was performed according to General Procedure C, using 0.20 g of PVCsolid. The reaction was performed twice.

The reaction with the PVC tubing was performed according to General Procedure C, using 0.30 g of PVCtubing (˜67% by wt PVC=˜0.20 g PVCsolid). A ˜2 inch piece of tubing was cut up in half lengthwise and then cut into smaller pieces that were added to the reaction (photo below). The PVCtubing pieces did not fully dissolve in the reaction mixture. The reaction was performed twice.

Once the plasticizer was removed, the resulting solid material (PVC_{solid}) had a Mₙ of 83 kg mol⁻¹ and was partially soluble in DMF. A 32% yield of Compound 2 (FIG. 6) was observed when the galvanostatic reaction was performed using PVC_{solid} alone (FIG. 5B), similar to the 29% yield observed previously with PVC₁₀₀ₖ alone. When PVC tubing was directly used—with the same total mass of polymer—the yield of Compound 2 substantially increased (to 63%). These results suggest that plasticized PVC waste can be used directly without needing to add more plasticizer. The dual-role of DEHP as plasticizer and redox mediator demonstrates that additives can serve one function during the lifetime of a plastic, and then be activated to serve a second function at the plastic's end-of-life. Moving forward, multipurpose plastic additives can be imagined that facilitate end-of-life treatment of plastics, which could considerably improve the overall efficiency of plastics reuse and repurposing.

### Example 13: Synthetic Procedures

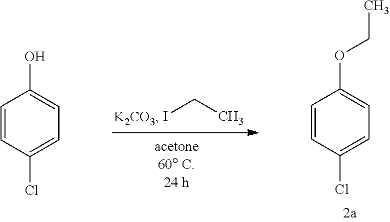

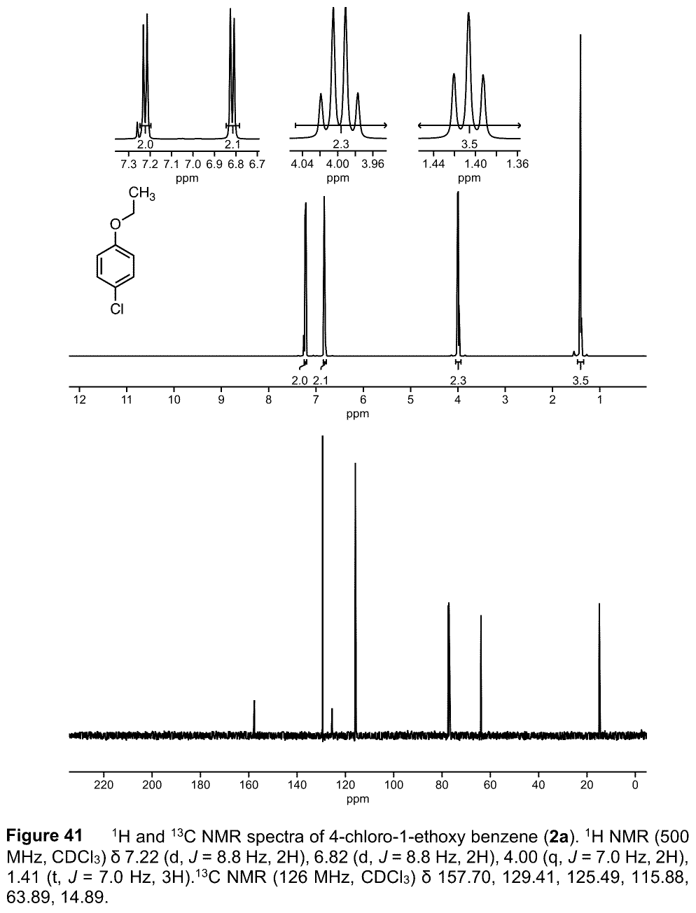

4-chloro-1-ethoxybenzene (2a). Potassium carbonate (2.57 g, 18.6 mmol, 1.2 equiv.) and 4-chlorophenol (1.99 g, 15.5 mmol, 1.0 equiv.) were added to a 50 mL round-bottom flask equipped with reflux condenser and dissolved in acetone (30 mL). Ethyl iodide (1.49 mL, 18.6 mmol, 1.2 equiv.) was added and the resulting solution was stirred at 60° C. After 24 h, the reaction was cooled to rt and concentrated under reduced pressure. The crude reaction mixture was directly purified by silica gel column chromatography (0-100% ethyl acetate in hexanes) to obtain a viscous, colorless oil (2.01 g, 83%). NMR data is reported in FIG. 41. HRMS (APCI+) calculated for C₉H₁₀ClO [M+H⁺]: 157.0415; found: 157.0423.

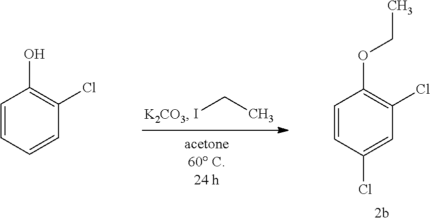

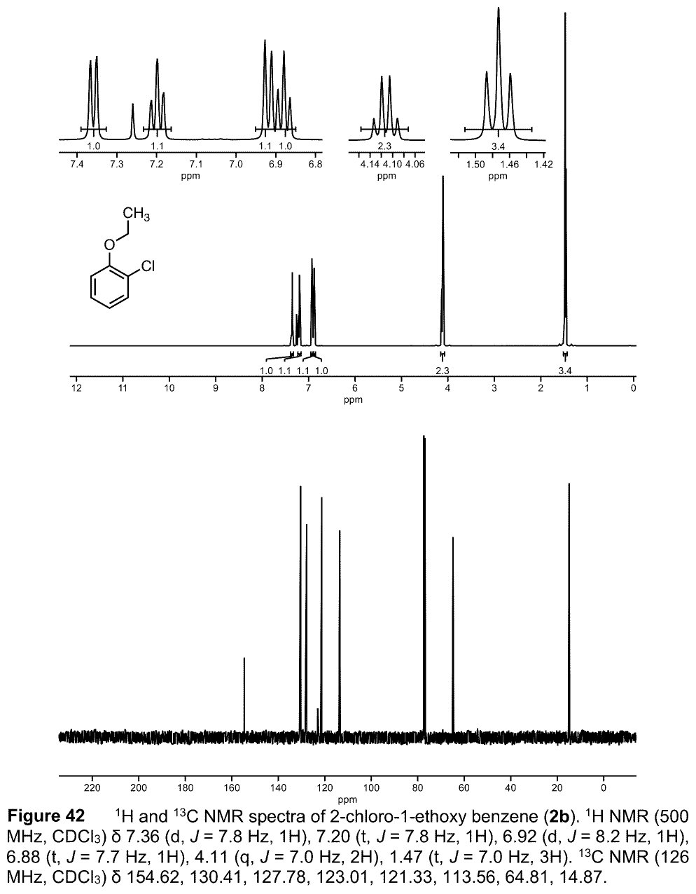

2-chloro-1-ethoxybenzene (2b). Potassium carbonate (2.57 g, 18.6 mmol, 1.2 equiv.) and 2-chlorophenol (1.99 g, 15.5 mmol, 1.0 equiv.) were added to a 50 mL round-bottom flask equipped with reflux condenser and dissolved in acetone (30 mL). Ethyl iodide (1.49 mL, 18.6 mmol, 1.2 equiv.) was added and the resulting solution was stirred at 60° C. After 24 h, the reaction was cooled to rt and concentrated under reduced pressure. The crude reaction mixture was directly purified by silica gel column chromatography (0-100% ethyl acetate in hexanes) to obtain and viscous, colorless oil (1.85 g, 76%). NMR data is reported in FIG. 42. HRMS (APCI+) calculated for C₉H₁₀ClO [M+H⁺]: 157.0415; found: 157.0424.

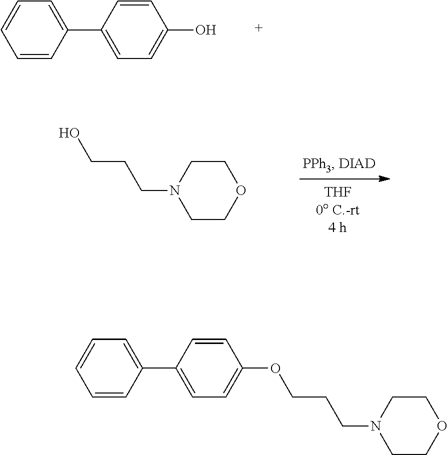 [SMILES: Oc1ccc(-c2ccccc2)cc1 ; OCCCN1CCOCC1 ; c1ccc(-c2ccc(OCCCN3CCOCC3)cc2)cc1]

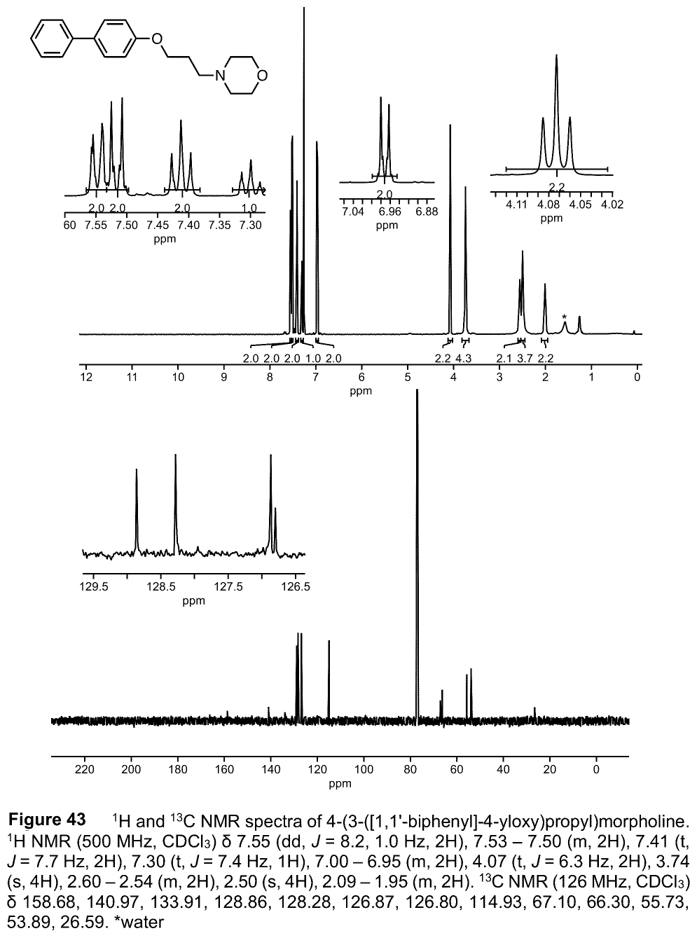

4-(3-([1,1′-biphenyl]-4-yloxy)propyl)morpholine. 4-Phenylphenol (1.00 g, 5.88 mmol), triphenylphosphine (1.85 g, 7.05 mmol), and 4-(3-hydroxypropyl)morpholine (0.975 mL, 7.05 mmol) were added to a round-bottom flask under N₂ and dissolved in anhydrous THF (17 mL). The reaction was cooled to 0° C. in an ice-bath and then diisopropyl azodicarboxylate (1.38 mL, 7.05 mmol) was added dropwise. The reaction was warmed for room temperature and stirred for a total of 4 h. After the reaction was complete, 50 mL of H₂O was added and then extracted 3×100 mL ethyl acetate. The combined organic layers were dried with MgSO₄, filtered, and concentrated under reduced pressure. The crude material was further purified by silica gel column chromatography (0-80% (ethyl acetate+5% triethylamine) in hexanes) to obtain a white solid (0.714 g, 2.40 mmol, 41%). NMR data is reported in FIG. 43. HRMS (EI+) calculated for C₁₉H₂₃NO₂ [M⁺]: 297.1729; found: 297.1726.

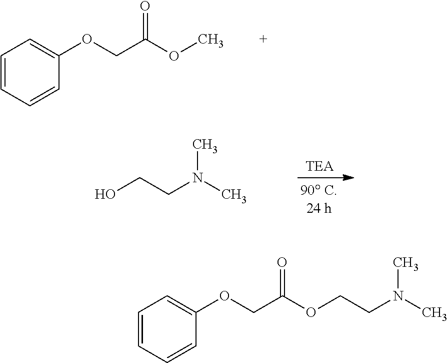

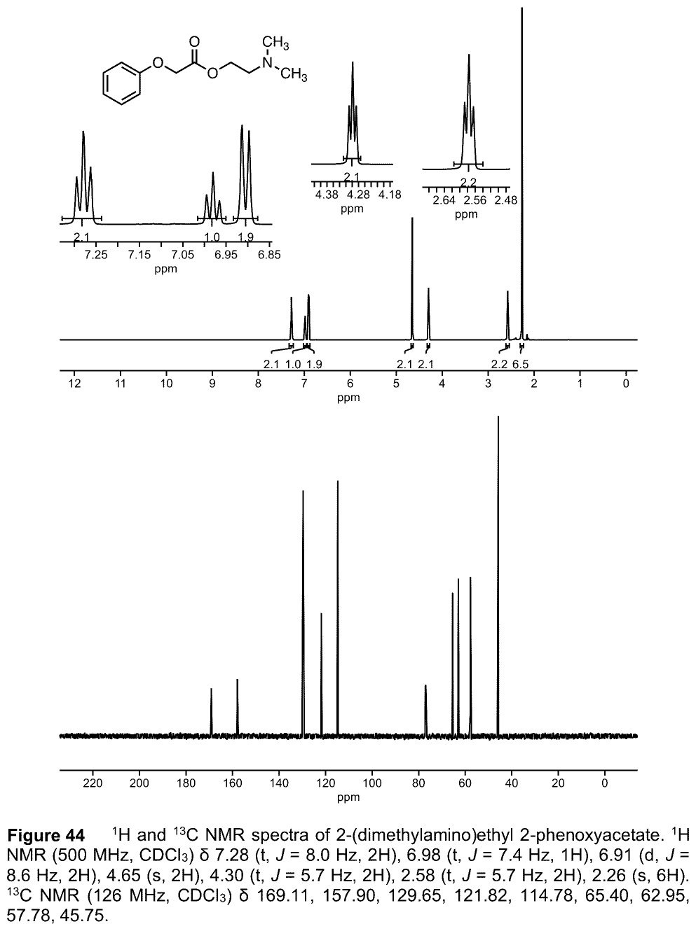

2-(dimethylamino)ethyl 2-phenoxyacetate. Methyl phenoxyacetate (1.00 mL, 1.15 g, 6.91 mmol), N,N-dimethylethanol amine (1.04 mL, 0.924 g, 10.4 mmol), and triethylamine (1 mL) were dispensed into an 8 mL glass vial. The reaction was stirred at 90° C. for 24 h. The crude reaction mixture was cooled to rt and directly purified by silica gel chromatography (0-50% (ethyl acetate+5% triethylamine) in hexanes) to obtain a colorless oil (1.23 g, 5.53 mmol, 80%). NMR data is reported in FIG. 44. HRMS (EI+) calculated for C₁₂H₁₇NO₃ [M⁺]: 223.1208; found: 223.1212.

### General Procedure E (Divided Cell Paired Electrolysis, Constant Current)

The ElectraSyn® Pro-Divide reaction vessel was assembled using graphite electrodes (0.8×0.2×5.20 cm) and a 5 micron frit separator layered with a PiperION anion-exchange membrane (pre-soaked in 0.1 M NBu₄BF₄ in DMF). The aromatic substrate (0.5 mmol), NBu₄BF₄ (231 mg, 0.70 mmol), and DMF (7 mL) were added to the working (anodic) chamber. PVC₄₇ₖ (200 mg, 3.2 mmol (repeat unit), 6.4 equiv. (repeat unit)), NBu₄BF₄ (231 g, 0.70 mmol), DEHP (0.20 mL, 0.50 mmol, 1.0 equiv.), and DMF (7 mL) were added to the counter (cathodic) chamber. All reagents were added under air and then dissolved by stirring for at least 15 min. Then the reaction mixture was subjected to constant current electrolysis (10 mA) at rt. The anodic reaction was monitored by GC and stirred until all the aromatic substrate was consumed. The working (anodic) chamber was poured into ˜250 mL of ethyl acetate and washed with H₂O (5×40 mL) and brine (1×50 mL) to remove DMF. The organic layer was dried with MgSO₄, filtered, and concentrated under reduced pressure. The crude reaction was further purified by column chromatography and the final product dried under reduced pressure.

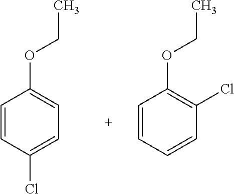

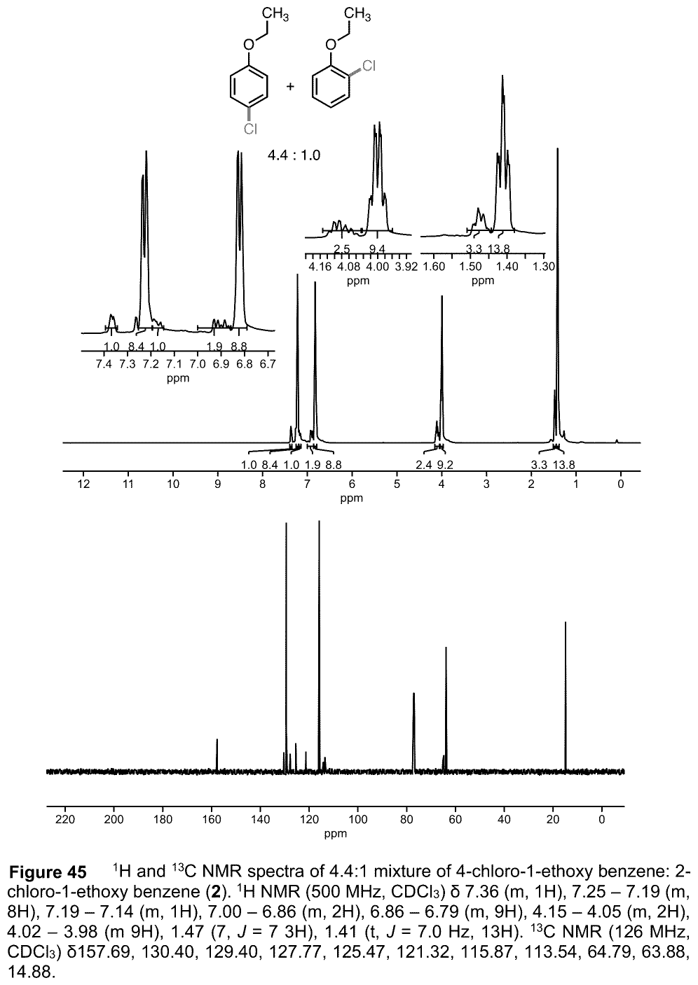

chloro-1-ethoxybenzene (2). 1-Ethoxybenzene (63 .iL, 61 mg, 0.50 mmol) was reacted according to General Procedure E for a total of 8 h. The crude product was purified by chromatography (mobile phase: 100% hexanes) to obtain a colorless oil (41 mg, 52% yield). NMR analysis indicated that the product was formed as a 4:1 mixture of regioisomers (4-chloro-1-ethoxybenzene: 2-chloro-1-ethoxybenzene). NMR data is reported in FIG. 45. HRMS (APCI+) calculated for C₉H₁₀ClO [M+H⁺]: 157.0415; found: 157.0422.

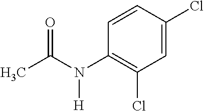

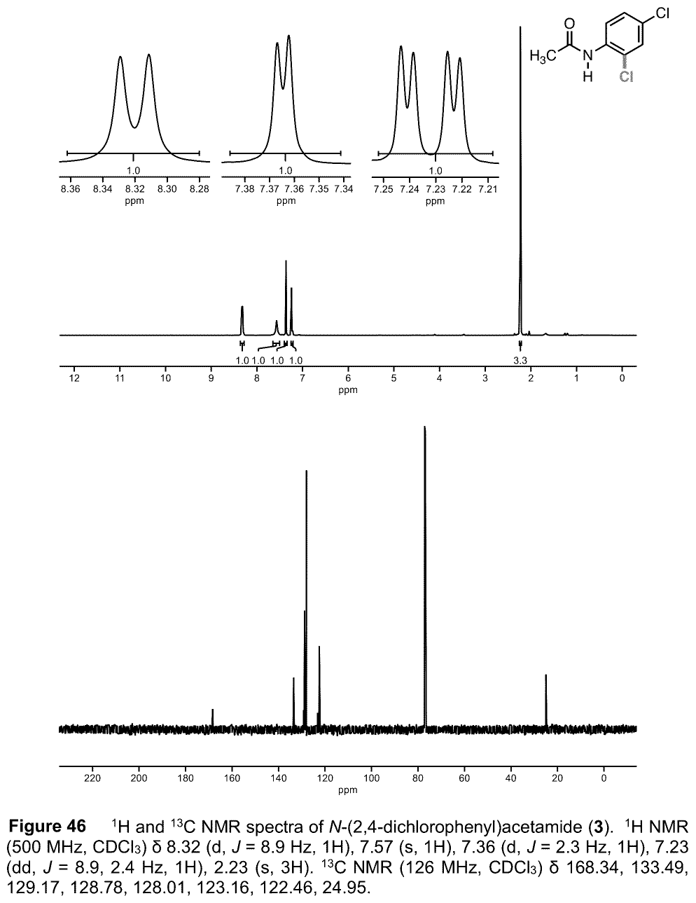

N-(2,4-dichlorophenyl)acetamide (3). N-(4-chlorophenyl)acetamide (85 mg, 0.50 mmol) was reacted according to General Procedure E for a total of 10 h. The crude product was purified by chromatography (mobile phase: 0-100% gradient ethyl acetate in hexanes) to obtain an off-white solid (69 mg, 67% yield). NMR data is reported in FIG. 46. HRMS (APCI+) calculated for C₈H₈Cl₂NO [M+H⁺]: 203.9977; found: 203.9984.

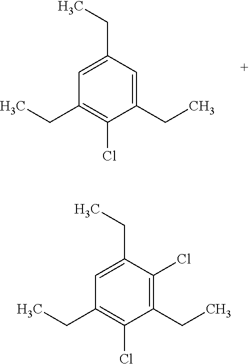

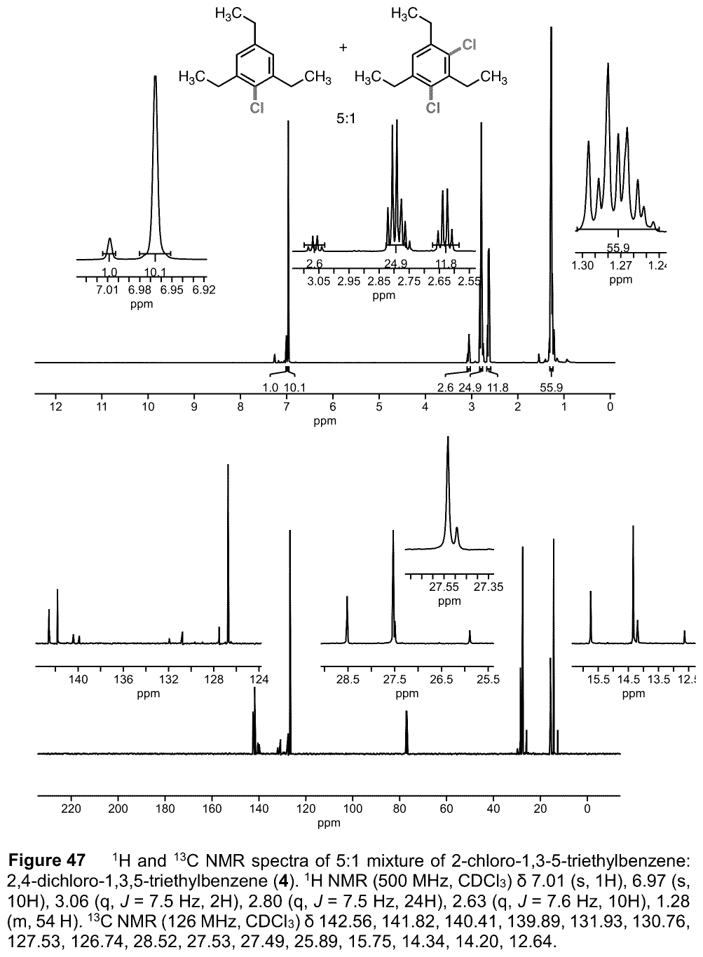

chloro-1,3,5-triethylbenzene (4). 1,3,5-Triethylbenzene (94 .iL, 81 mg, 0.50 mmol) was reacted according to General Procedure E for a total of 8 h. The crude product was purified by chromatography (mobile phase: 100% hexanes) to obtain a colorless liquid (77 mg, 76% yield). NMR analysis indicated that the product was formed as a 5:1 mixture of products (2-chloro-1,3-5-triethylbenzene: 2,4-dichloro-1,3,5-triethylbenzene). NMR data is reported in FIG. 47. HRMS (EI+) calculated for C₁₂H₁₇Cl: 196.1019 [M⁺]; found: 196.1022; calculated for C₁₂H₁₆Cl₂ [M⁺]: 230.0629; found: 230.0629.

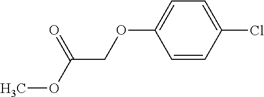

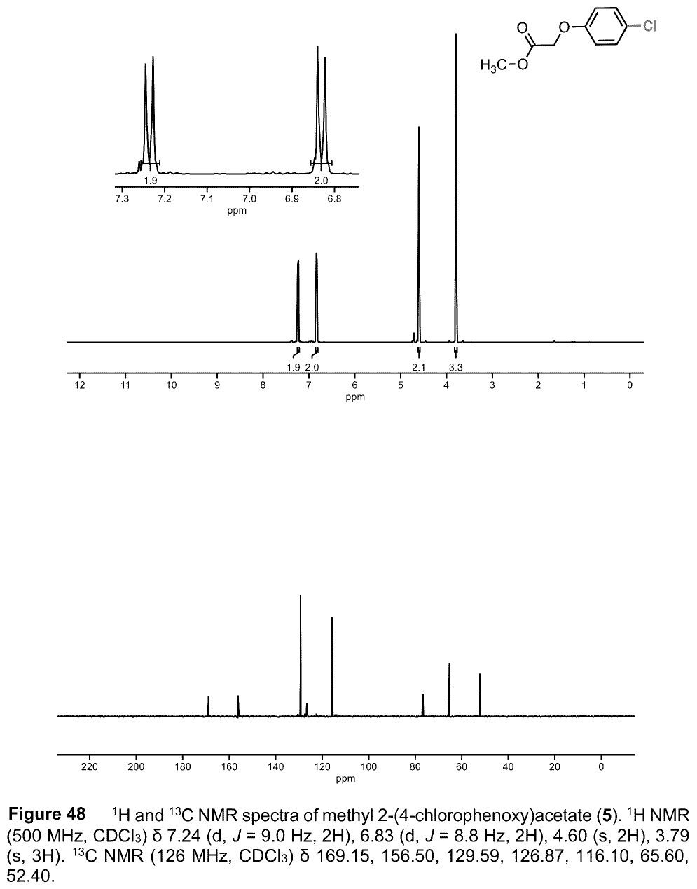

methyl 2-(4-chlorophenoxy)acetate (5). Methyl phenoxy acetate (72 .iL, 83 mg, 0.50 mmol) was reacted according to General Procedure E for a total of 7 h. The crude product was purified by chromatography (mobile phase: 15% ethyl acetate in hexanes) to obtain a yellow oil (72 mg, 72% yield). NMR data is reported in FIG. 48. HRMS (APCI+) calculated for C₉H₁₀ClO₃ [M+H⁺]: 201.0313; found: 201.0322.

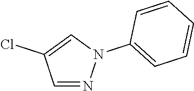 [SMILES: Clc1cnn(-c2ccccc2)c1]

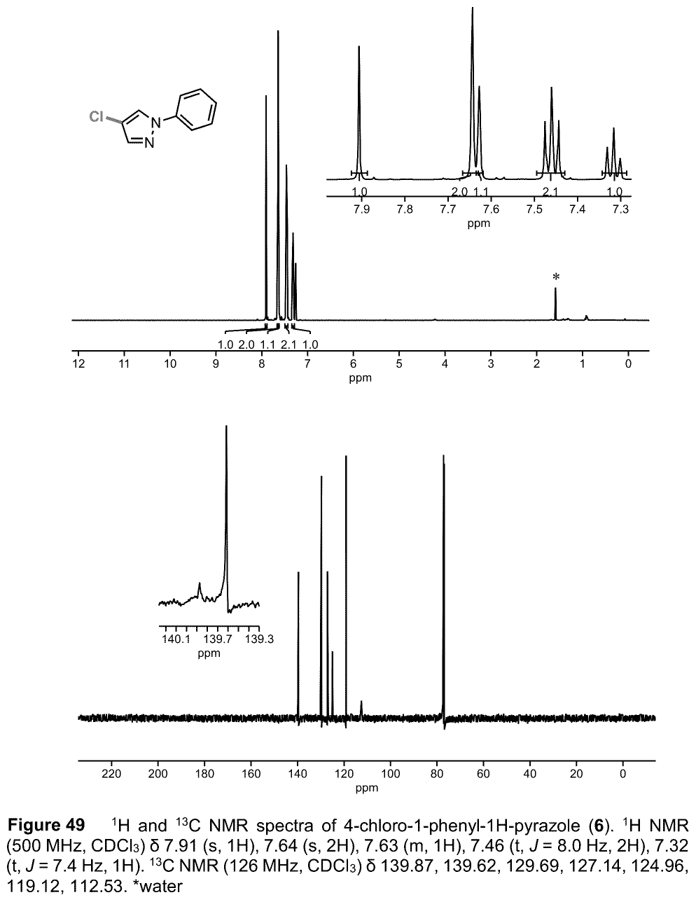

4-chloro-1-phenyl-1H-pyrazole (6). 1-Phenyl-1H-pyrazole (66 .iL, 72 mg, 0.50 mmol) was reacted according to General Procedure E for a total of 8 h. The crude product was purified by chromatography (mobile phase: 0-30% gradient ethyl acetate in hexanes) to obtain an off-white solid (64 mg, 71% yield). NMR data is reported in FIG. 49. HRMS (EI+) calculated for C₉H₇ClN₂ [M⁺]: 178.0298; found: 178.0300.

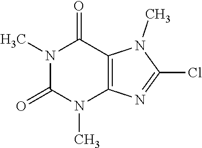

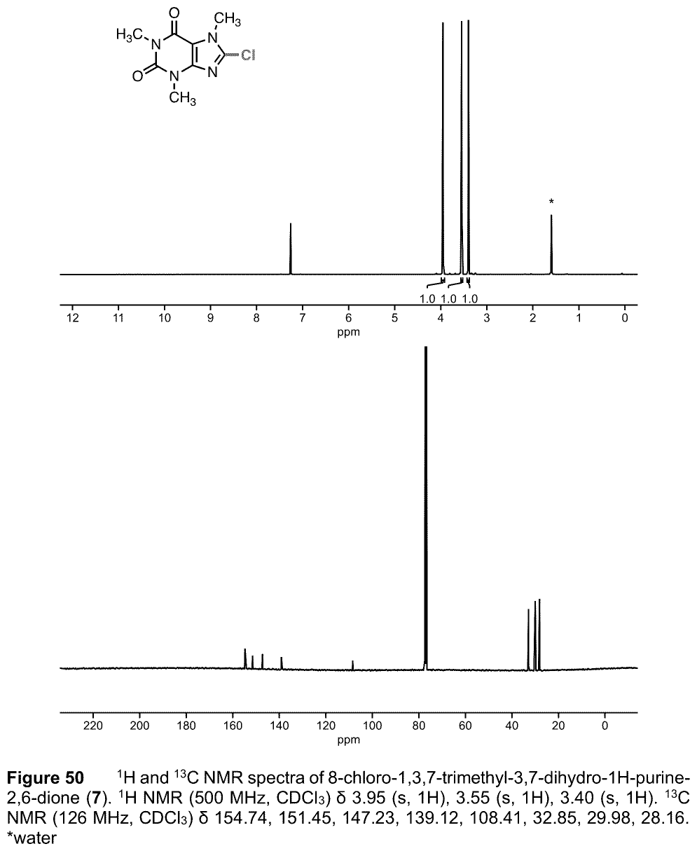

8-chloro-1,3,7-trimethyl-3,7-dihydro-1H-purine-2,6-dione (7). Caffeine (97 mg, 0.50 mmol) was reacted according to General Procedure E for a total of 8 h. The crude product was purified by chromatography (mobile phase: 0-50% ethyl acetate in hexanes) to obtain a white solid (41 mg, 36% yield). NMR data is reported in FIG. 50. HRMS (APCI+) calculated for C₈H₁₀ClN₄O₂ [M+H⁺]: 229.0487; found: 229.0482.

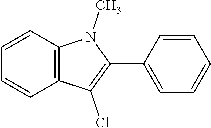 [SMILES: Cn1c(-c2ccccc2)c(Cl)c2ccccc21]

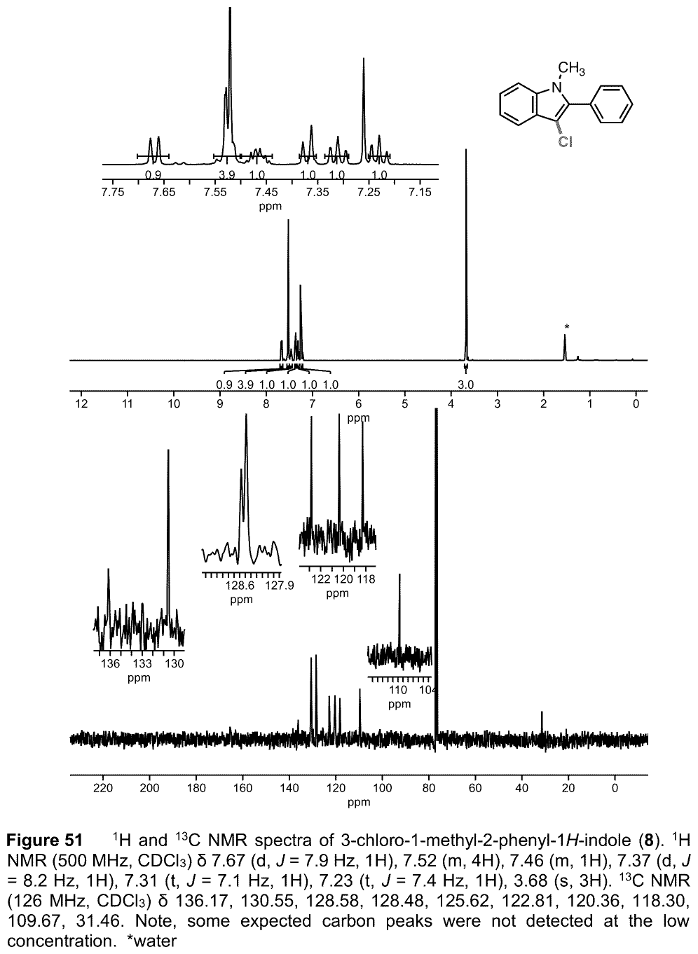

3-chloro-1-methyl-2-phenyl-1H-indole (8). 1-Methyl-2-phenyl-1H-indole (104 mg, 0.50 mmol) was reacted according to General Procedure E for a total of 4 h. The crude product was purified by chromatography (mobile phase: 100% hexanes) to obtain a white solid (6.1 mg, 5% yield). NMR data is reported in FIG. 51. HRMS (EI+) calculated for C₁₅H₁₂ClN [M⁺]: 241.0658; found: 241.0664.

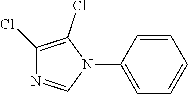 [SMILES: Clc1ncn(-c2ccccc2)c1Cl]

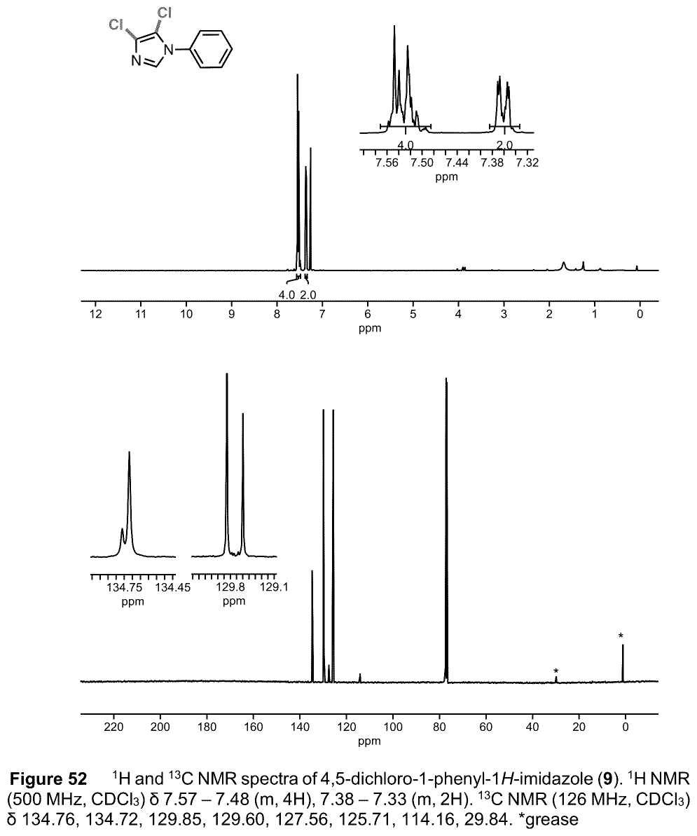

4,5-dichloro-1-phenyl-1H-imidazole (9). 1-Phenyl-1H-imidazole (63 .iL, 72 mg, 0.50 mmol) was reacted according to General Procedure E for a total of 4 hours. The crude product was purified by chromatography (mobile phase: 0-100% gradient ethyl acetate in hexanes) to obtain a white solid (14 mg, 13% yield). NMR data is reported in FIG. 52. HRMS (EI+) calculated for C₉H₆Cl₂N₂ [M⁺]: 211.9908; found: 211.9907.

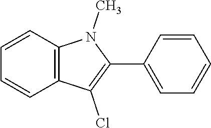 [SMILES: Cn1c(-c2ccccc2)c(Cl)c2ccccc21]

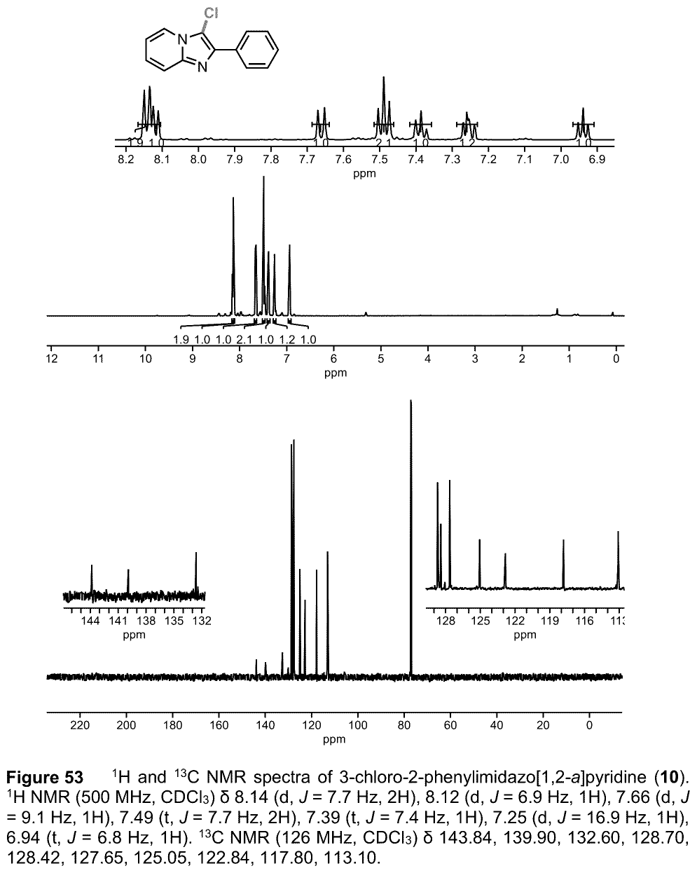

3-chloro-2-phenylimidazo[1,2-a]pyridine (10). 2-Phenylimidazo[1,2-a]pyridine (97 mg, 0.50 mmol) was reacted according to General Procedure E for a total of 4 h. The crude product was purified by chromatography (mobile phase: 0-100% gradient ethyl acetate in hexanes) to obtain an off-white solid (27 mg, 24% yield). NMR data is reported in FIG. 53. HRMS (APCI+) calculated for C₁₃H₁₀ClN₂ [M+H⁺]: 229.0527; found: 229.0526.

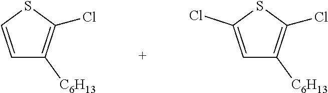

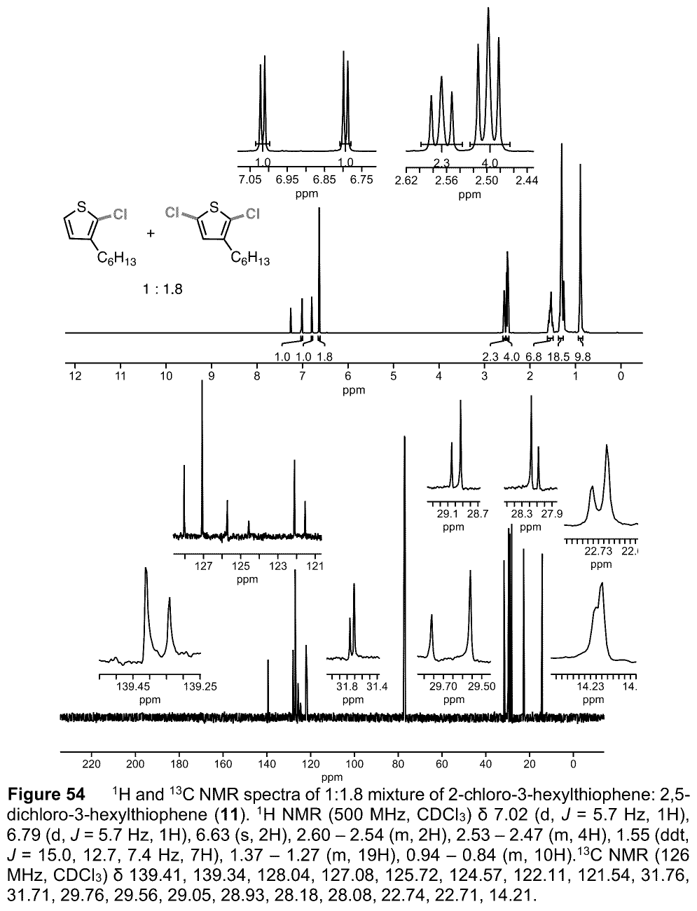

chloro-3-hexylthiophene (11). 3-Hexylthiophene (89 μL, 84 mg, 0.50 mmol) was reacted according to General Procedure E for a total of 8 h. The crude product was purified by chromatography (mobile phase: 100% hexanes) to obtain a colorless oil (37 mg, 33% yield). NMR analysis indicated that the product was formed as a 1:1.8 mixture of products (2-chloro-3-hexylthiophene: 2,5-dichloro-3-hexylthiophene). NMR data is reported in FIG. 54. HRMS (APCI+) calculated for C₁₀H₁₆ClS [M+H⁺]: 203.0656; found: 203.0655; C₁₀H₁₅Cl₂S [M+H⁺]: 237.0266; found: 237.0263.

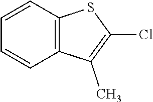

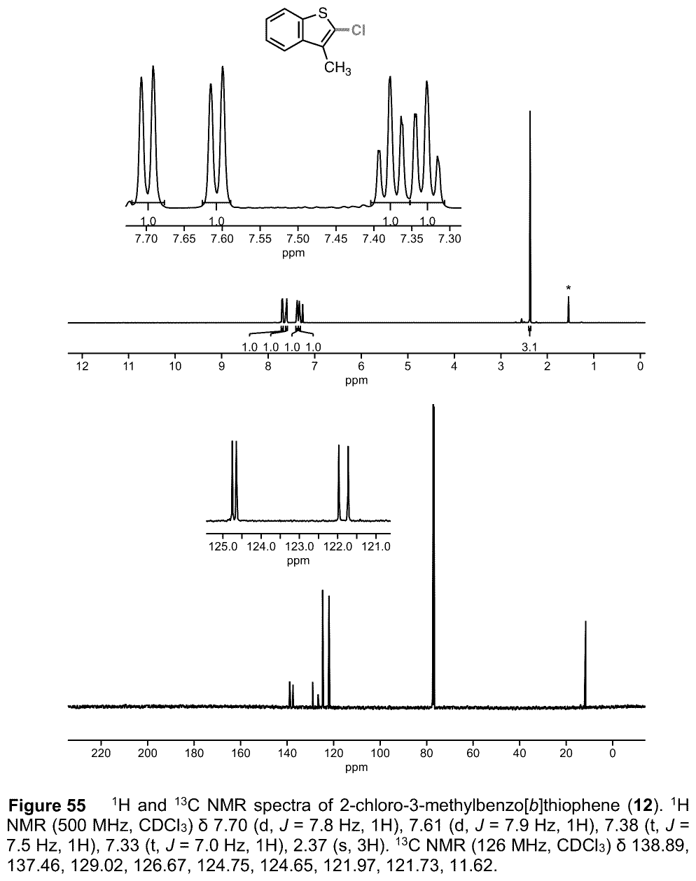

2-chloro-3-methylbenzo[b]thiophene (12). 3-Methylbenzo[b]thiophene (67 .iL, 74 mg, 0.50 mmol) was reacted according to General Procedure E for a total of 10 h. The crude product was purified by chromatography (mobile phase: 100% hexanes) to obtain a colorless oil (50 mg, 55% yield. NMR data is reported in FIG. 55. HRMS (EI+) calculated for C₉H₇ClS [M⁺]: 181.9957; found: 181.9888.

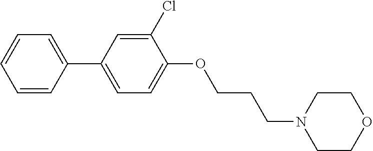 [SMILES: Clc1cc(-c2ccccc2)ccc1OCCCN1CCOCC1]

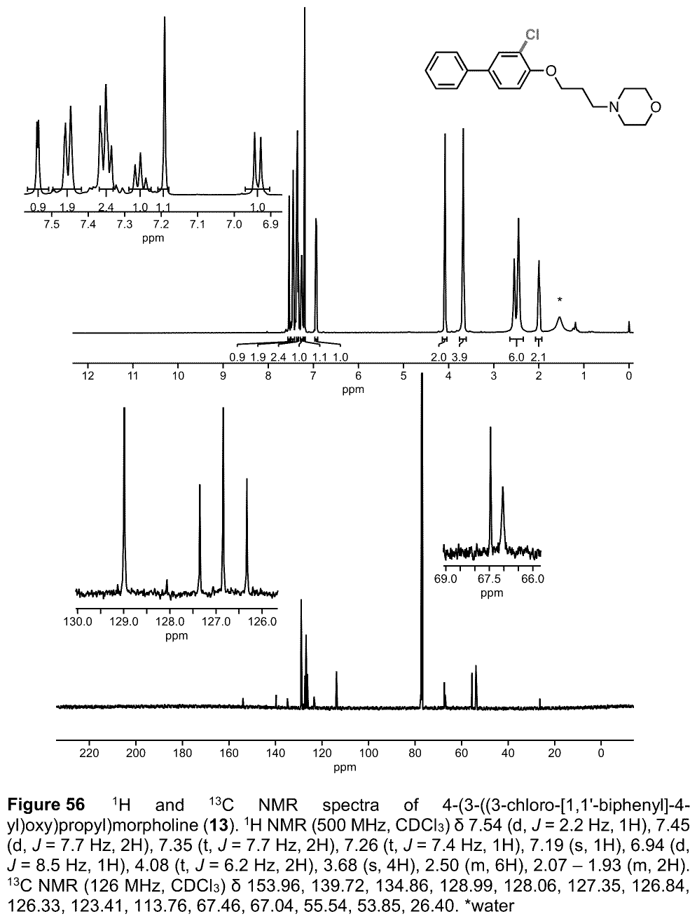

4-(3-((3-chloro-[1,1′-biphenyl]-4-yl)oxy)propyl)morpholine (13). 4-(3-([1,1′-Biphenyl]-4-yloxy)propyl)morpholine (0.149 g, 0.50 mmol) was reacted according to General Procedure E for a total of 8 h. The crude product was purified by chromatography (mobile phase: 0-80% (ethyl acetate+5% triethylamine)) to obtain and off-white solid (73 mg, 44% yield). NMR data is reported in FIG. 56. HRMS (EI+) calculated for C₁₉H₂₂ClNO₂ [M⁺]: 331.1339; found: 331.1336.

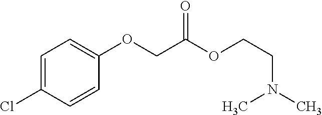 [SMILES: CN(C)CCOC(=O)COc1ccc(Cl)cc1]

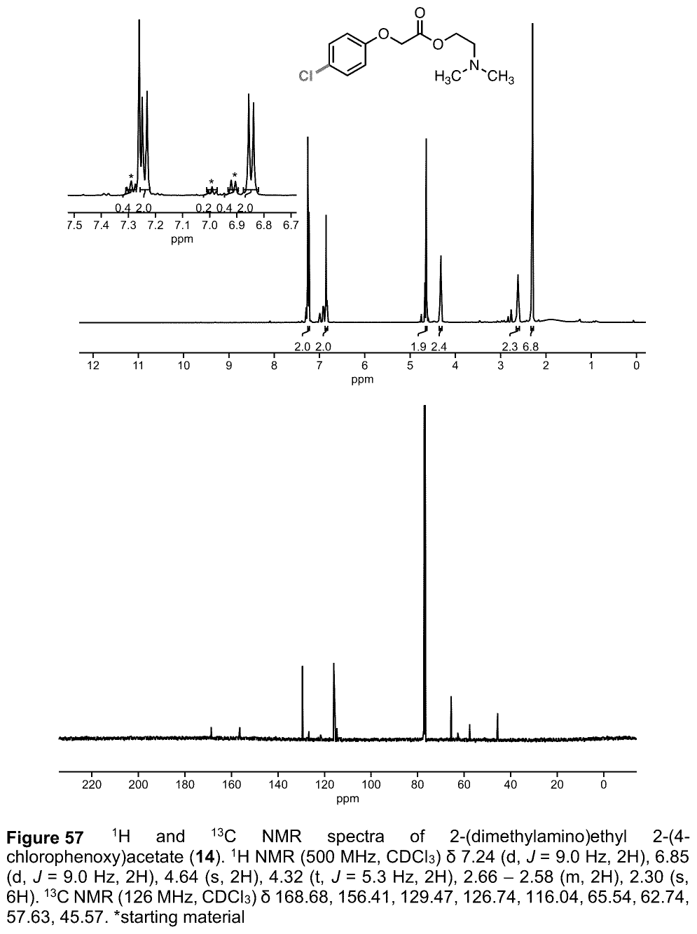

2-(dimethylamino)ethyl 2-(4-chlorophenoxy)acetate (14). 2-(Dimethylamino)ethyl 2-phenoxyacetate (107 .iL, 0.112 g, 0.5 mmol) was reacted according to General Procedure E for a total of 8 h. The crude product was purified by chromatography (mobile phase: 0-60% (ethyl acetate+5% triethylamine)) to obtain a yellow oil (44 mg, 29% yield). NMR analysis indicated that the obtained oil contained 1:0.2 mixture of product and unreacted starting material, which were not separated chromatographically. NMR data is reported in FIG. 57.

### Example 14: Cyclic Voltammetry Screening of Redox Active Organic Compounds

Cyclic voltammetry (CV) was used to screen various redox active organic compounds ranging from aromatic to hetero-aromatic scaffolds. It was observed that organic compounds having a reduction potential of −1.6 V Ag/Ag⁺ or lower could be suitable as a redox mediator for methods of the disclosure. CV was done with glassy carbon working electrode, Pt-wire counter electrode and Ag/Ag⁺ reference electrode in 0.3 M NBu₄BF₄ in DMF with 10 mM of the organic molecule at 100 mV/s. Reduction potential (E_{1/2}) was reported with reference to Ag/Ag⁺. For analyzing the mediated properties, 30 mM PVC₂₂ₖ was added to the CV reaction vial to observe increase in reduction current peaks with PVC. Reduction potential of the redox mediators are summarized below.

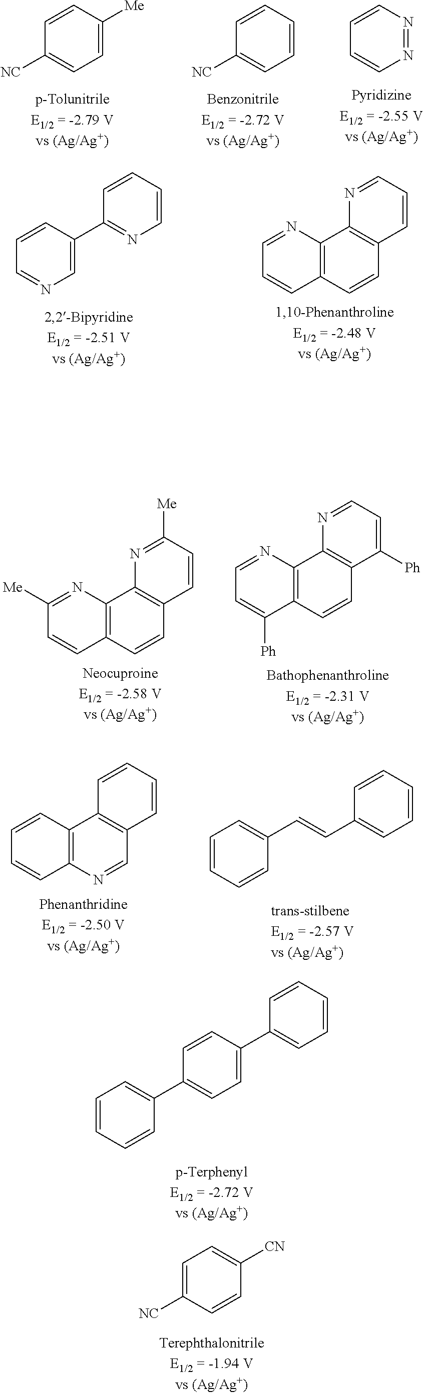

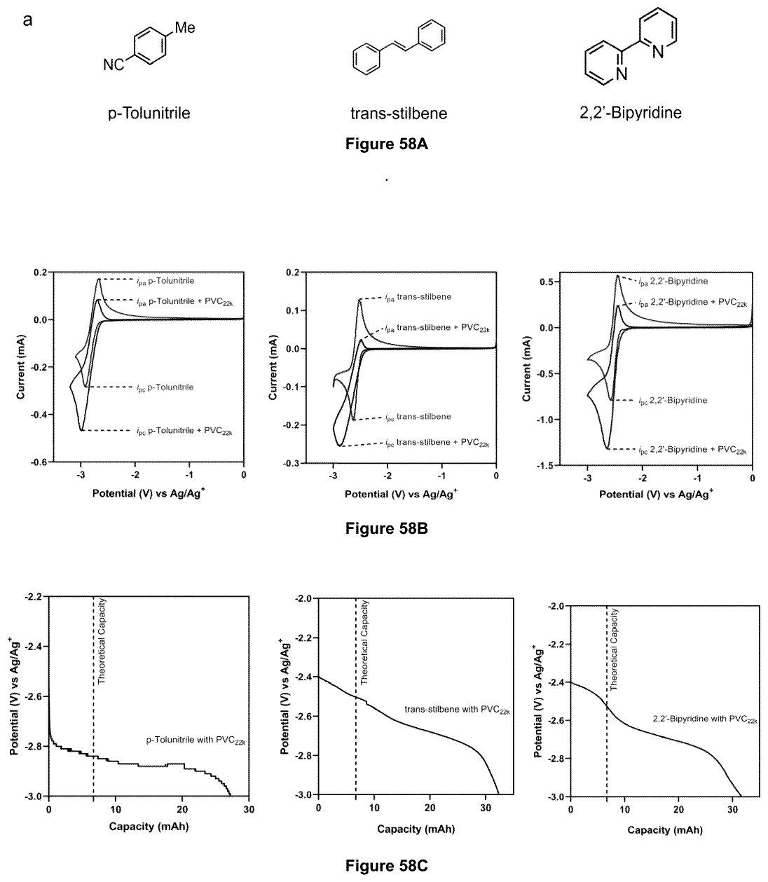

Referring to FIGS. 58A, p-Tolunitrile, 2,2′Bipyridine, and trans-stilbene showed promising results as potential redox mediators for PVC reduction. Referring to FIG. 58B, cyclic voltammograms of these compounds with PVC shows increment in cathodic current and decreased reversibility of reduction peaks, indicating electron transfer to PVC, and as a consequence lower oxidation current. Evidence of mediated behavior was analyzed by constant-current bulk electrolysis with p-Tolunitrile/2,2-Bipyridine/trans-stilbene with PVC at the cathode/working electrode and phenetole at the anode/counter electrode. All three compounds showed substantial increment (˜4-5 times higher) in capacity as compared to the theoretical capacity (6.7 mA h), indicating that the presence of PVC generated a greater number of electron than these redox active compounds (FIG. 58C).

### Example 15: Mediator-Free Process

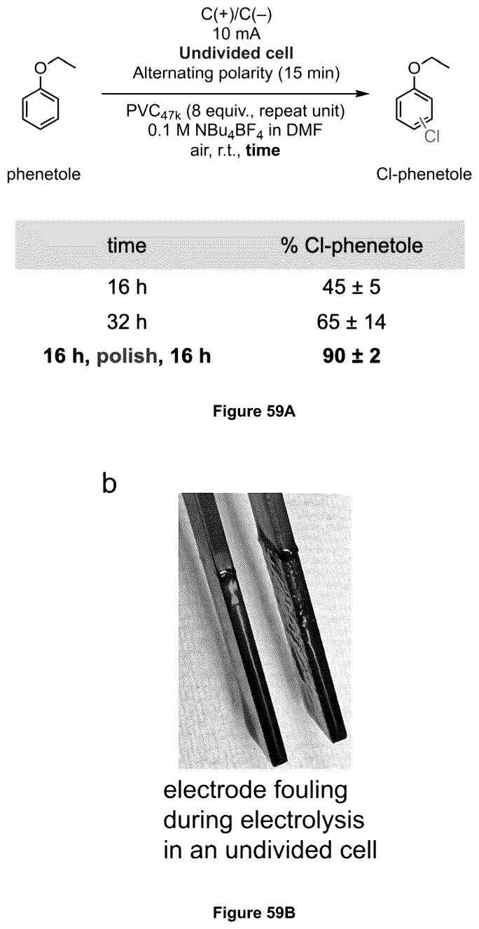

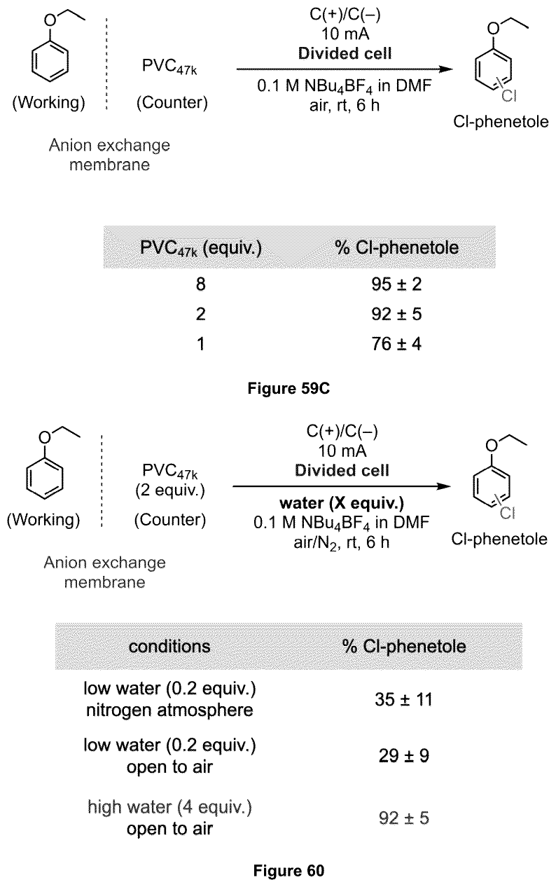

Referring to FIG. 59A, a mediator-free process was evaluated first in an undivided cell with waste PVC and phenetole as the organic compound to be chlorinated. A paired-electrolysis process was performed in an undivided cell with alternating polarity every 15 minutes to minimize polymer film formation on the cathode. The process in the undivided cell did not improve the yield of chlorinated arenes beyond 50% in 16 hours. Doubling the reaction time increased the yield up to 60%. It was observed that film formation on the anode slowed down the chlorinate of arenes. FIG. 59B is an image showing the electrode fouling. The reaction was paused after 16 h and the electrodes were polished before resuming the reaction for another 16 h with the cleaned electrodes. This resulted in a yield of up to 90%.

To restrict anode fouling and reduce reaction time, the process was performed in a divided cell in which the anode and cathode were separated by an anion exchange membrane. That reaction gave >90% Cl-phenetole. This process also allowed for lowering the PVC equivalence from 8 equiv. to 1 equiv. to improve the atom economy, while still maintaining a good yield. As shown in FIG. 59C, the process with 2 equiv PVC provided a 92% Cl-phenetole and reduction further to 1 equiv PVC still provided a 76% Cl-phenetole.

To assess the impact of water and oxygen on electrolysis, control reactions with phenetole as a model arene in a divided cell with low and high water concentration in air and nitrogen atmosphere were performed. Referring to FIG. 60, under the same conditions of low water concentration (0.2 equiv.), the yield of Cl-phenetole was similar in the air and in the nitrogen atmosphere, which indicates oxygen is not affecting the rate of arene chlorination significantly. However, at high water concentration (4 equiv.), a higher yield of Cl-phenetole (>90%) shows the pivotal role of water in the reaction. Without intending to be bound by theory, it is believed this higher yield could be attributed to hydroxide ions generated from residual water reduction that may act as a base, deprotonating PVC to form dehydrochlorinated PVC and producing chloride ions that can participate in phenetole chlorination.

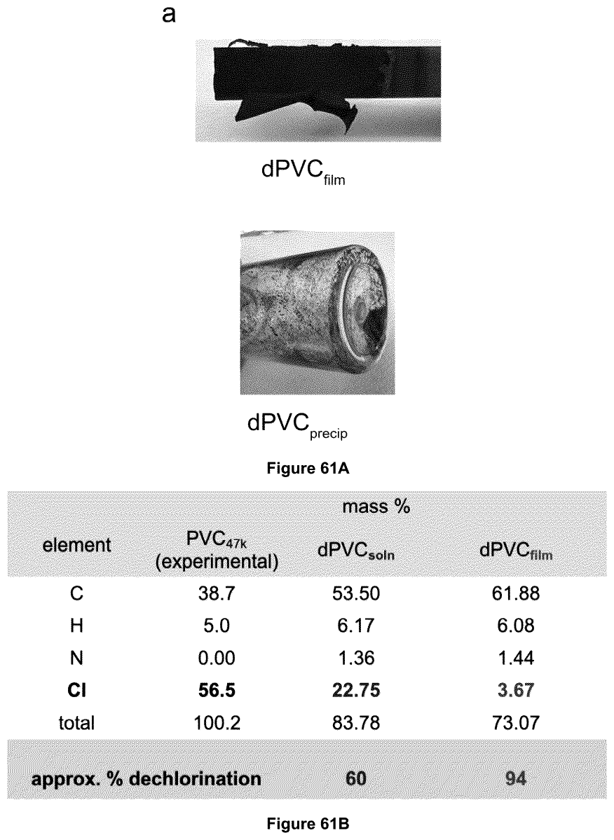

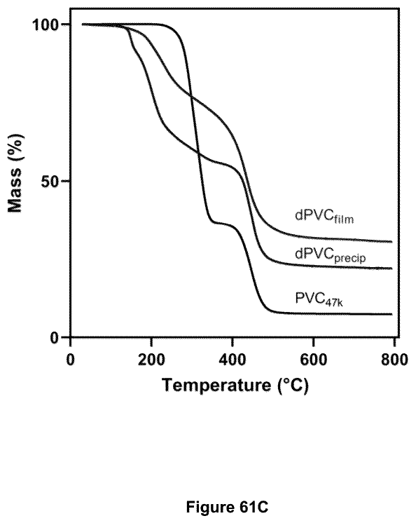

Referring to FIG. 61A, after the electrolysis, two types of dPVC were obtained—a film deposited on the cathode (dPVC_{film}) and a precipitate in solution (dPVC_{precip}). Both forms of the polymer were insoluble in most of the conventional solvents at room temperature and at high temperatures up to 135° C. Without intending to be bound by theory, it is believed that this can be indicative of formation of polyacetylene and/or crosslinks on the PVC backbone. Referring to FIG. 61B, elemental analysis showed a higher degree of dechlorination for dPVC_{film} (95%) as compared to dPVC_{precip} (60%). Without intending to be bound by theory, it is believed that the higher degree of dechlorination of the film can be due to its proximity to the cathode. The high degree of dechlorination indicates a near-chlorine free polymer backbone, which can be used safely for pyrolysis and incineration.

Referring to FIG. 61C, thermogravimetric analysis (TGA) was used to determine the relative amount of chlorine-containing repeat units in dPVC_{film} and dPVC_{precip}. The thermal loss profile of PVC shows two main steps—a first step which has main loss at about 300° C., where mostly HCl was generated along with some hydrocarbon degradation products, and second step where remaining hydrocarbon content degrades at about 450° C. In the first step, both the dPVC_{film} and dPVC_{precip} showed lower mass loss as compared to the starting material PVC₄₇ₖ, indicating lower chlorine content in the dPVCs. More than 30% mass residue left at 800° C., which is indicative of char formation.

The resulting dPVCs can be used in a variety of ways. In the case of partial dechlorination, the dPVC can be repurposed as a surrogate for elastomers. Hydrogenation can alternatively lead to a chlorinated polyethylene mimic. A complete dechlorination can results in materials that can be useful as conductive polymers. Metathesis may be used to break the resulting polymer into smaller olefin fragments for potential application of waxes or fuels.

### Aspects

Aspects 1. A method for halogenation of an organic compound with a reactive halogen species generated from a waste source comprising at least one halogen containing polymer, comprising:
admixing the waste source with the organic compound, a solvent, and an electrolyte, wherein the solvent swells and/or dissolves the halogen containing polymer in the waste source; and
exposing the admixture to a negative voltage in an electrochemical cell comprising a cathode and an oppositely disposed anode, wherein:
the halogen containing polymer is dehalogenated by the cathode to generate a halogen anion;
the halogen anion is formed into the reactive halogen species by the anode; and
the halogen species reacts with the organic compound to halogenate the organic compound.

Aspects 2. A method for halogenation of an organic compound with a reactive halogen species from a waste source comprising at least one halogen containing polymer, comprising:
admixing the waste source with a solvent, and an electrolyte, wherein the solvent swells and/or dissolves the halogen containing polymer in the waste source;
exposing the admixture to a negative voltage in an electrochemical cell comprising a cathode by which the halogen containing polymer is dehalogenated and halogen anions are generated, and an oppositely disposed anode by which the halogen anions are transformed into the reactive halogen species;
recovering the reactive halogen species from the electrochemical cell; and
admixing the reactive halogen species with the organic compound, wherein the reactive halogen species reacts with the organic compound to halogenate the organic compound.

Aspects 3. The method of claim 2, wherein the admixing of the reactive halogen species with the organic compound is performed in a container physically separated from the electrochemical cell.

Aspects 4. The method of claim 2 or 3, wherein recovering the reactive halogen species comprises removing the reactive halogen species from the electrochemical cell.

Aspects 5. A method for halogenation of an organic compound with a reactive halogen species from a waste source comprising at least one halogen containing polymer, comprising:
admixing the waste source with a solvent and an electrolyte, wherein the solvent swells and/or dissolves the halogen containing polymer in the waste source;
introducing the admixture comprising the waste source into a divided electrochemical cells comprising an anode and an oppositely disposed cathode separated from the anode by an anion exchange membrane, wherein the admixture comprising the waste source is introduced into a cathode-side of the divided electrochemical cell;
introducing an organic compound to be halogenated on an anode-side of the divided electrochemical cell;
applying a negative voltage to the admixture comprising the waste source in the electrochemical cell, whereby the halogen containing polymer is dehalogenated and halogen anions are generated and flow through the anion exchange membrane to the anode wherein the halogen anions are converted into the reactive halogen species,
wherein the reactive halogen species reacts with the organic compound present in the anode-side of the divided electrochemical cell to halogenate the organic compound.

Aspects 6. The method of any one of any one of the preceding claims, wherein the admixture comprises a redox mediator added in an amount of 0 equiv. to about 10 equiv relative to an amount of halogen containing polymer repeat unit present in the waste source.

Aspects 7. The method of claim 6, wherein the admixture further comprises the redox mediator present in an amount of greater than 0 equiv to about 10 equiv. relative to an amount of halogen containing polymer repeat unit present in the waste source.

Aspects 8. The method of claim 6 or 7, wherein the redox mediator reduces as a voltage of −1V verses Ag/Ag⁺ or at more negative potentials.

Aspects 9. The method of any one of claims 6 to 8, wherein the redox mediator is one or more phthalates.

Aspects 10. The method of any one of claims 6 to 9, wherein the redox mediator is one or more of p-tolunitrile, terephthalonitrile, benzonitrile, pyridazine, 1, 10-phenanthroline, neocuproine, bathophenanthroline, phenanthridine, 2,2′-bipyridine, trans-stilbene, and p-terphenyl.

Aspects 11. The method of any one of the preceding claims, wherein the waste source comprises a plasticizer.

Aspects 12. The method of claim 11, wherein the plasticizer comprises a phthalate.

Aspects 13. The method of any one of the preceding claims, wherein the solvent comprises one or more of dimethylformamide, acetonitrile, propylene carbonate, dimethoxyethane.

Aspects 14. The method of any one of the preceding claims, wherein the electrolyte comprises tetrabutylammonium tetrafluoroborate, tetrabutylammonium hexafluorophosphate, tetrabutylammonium bis(trifluoromethanesulfonyl)imide.

Aspects 15. The method of any one of the preceding claims, wherein the cathode and the anode are formed of a material independently selected from graphite, gold, non-graphite carbon-based electrodes, and platinum.

Aspects 16. The method of any one of the preceding claims, wherein the waste source comprises one or more chloride containing polymers.

Aspects 17. The method of claim 16, wherein the chloride containing polymer is PVC and/or PVDC.

Aspects 18. The method of any one of the preceding claims, wherein the waste source comprises one or more fluorinated polymers.

Aspects 19. The method of claim 18, wherein the fluorinated polymers comprise one or more of PVDF, PVF, and Teflon.

Aspects 20. The method of any one of the preceding claims, wherein the waste source is a mixed plastic waste source comprising the at least one halogen containing polymer.

Aspects 21. The method of any one of the preceding claims, wherein the at least one halogen containing polymer comprises F, Cl, Br, and/or I.

Aspects 22. The method of claim 1, 2 or 5, wherein the admixture is free of a redox mediator.

Aspects 23. The method of claim 1, 2, or 5, wherein the admixture is free of an added redox mediator.

Aspects 24. The method of any one of the preceding claims, wherein the admixture comprising the waste source comprises an amount of the waste source such that 1 equiv to 10 equiv of the halogen containing polymer repeat unit is present in the admixture relative to an amount of the organic compound.

Aspects 25. The method of any one of the preceding claims, wherein the voltage is about −1 V vs Ag/Ag⁺ to about −3 V vs Ag/Ag⁺.

Aspects 26. The method of any one of the preceding claims, wherein the solvent comprises water.

The foregoing description is given for clearness of understanding only, and no unnecessary limitations should be understood therefrom, as modifications within the scope of the disclosure may be apparent to those having ordinary skill in the art.

All patents, patent applications, government publications, government regulations, and literature references cited in this specification are hereby incorporated herein by reference in their entirety. In the case of conflict, the present description, including definitions, will control.

Throughout the specification, where the compounds, compositions, methods, and/or processes are described as including components, steps, or materials, it is contemplated that the compounds, compositions, methods, and/or processes can also comprise, consist essentially of, or consist of any combination of the recited components or materials, unless described otherwise. Component concentrations can be expressed in terms of weight concentrations, unless specifically indicated otherwise. Combinations of components are contemplated to include homogeneous and/or heterogeneous mixtures, as would be understood by a person of ordinary skill in the art in view of the foregoing disclosure.
Pud, A. A.; Rogalsky, S. P.; Shapoval, G. S. Reactions at the lower potential limit in aprotic medium at a platinum cathode revisited: their role in indirect electrochemical reductive degradation of polymers. J. Electroanal. Chem. 2000, 480, 1-8.
Bi, K.; Weathers, A.; Matsushita, S.; Pettes, M. T.; Goh, M.; Akagi, K.; Shi, L. Iodine doping effects on the lattice thermal conductivity of oxidized polyacetylene nanofibers. J. Appl. Phys. 2013, 114, 194302.
https://auroraplastics.com/materials/cpe-alloys/.
Basescu, N.; Liu, Z.-X.; Moses, D.; Heeger, A. J.; Naarmann, H.; Theophilou, N. High electrical conductivity in doped polyacetylene. Nature 1987, 327, 403-405.
Sytniczuk, A.; Dabrowski, M.; Banach, L.; Urban, M.; Czarnocka-sniadala, S.; Milewski, M.; Kajetanowicz, A.; Grela, K. At long last: olefin metathesis macrocyclization at high concentration. J. Am. Chem. Soc. 2018, 140, 8895-8901.

What is claimed:

1. A method for halogenation of an organic compound with a reactive halogen species generated from a waste source comprising at least one halogen containing polymer, comprising:
admixing the waste source with the organic compound, a solvent, and an electrolyte, wherein the solvent swells and/or dissolves the halogen containing polymer in the waste source; and
exposing the admixture to a negative voltage in an electrochemical cell comprising a cathode and an oppositely disposed anode, wherein:
the halogen containing polymer is dehalogenated by the cathode to generate a halogen anion;
the halogen anion is formed into the reactive halogen species by the anode; and
the halogen species reacts with the organic compound to halogenate the organic compound.

2. The method of claim 1, wherein the admixture further comprises a redox mediator.

3. The method of claim 2, wherein the redox mediator is one or more phthalates.

4. The method of claim 2, wherein the redox mediator is added in an amount of 0 equiv. to about 10 equiv.

5. The method of claim 1, wherein the waste source comprises a plasticizer.

6. The method of claim 1, wherein the waste source comprises one or more of PVC and PVDC, or wherein the waste source comprises one or more of PVDF, PVF, and Teflon.

7. The method of claim 1, wherein the waste source is a mixed plastic waste source comprising the at least one halogen containing polymer.

8. The method of claim 1, wherein the at least one halogen containing polymer comprises F, Cl, Br, and/or I.

9. The method of claim 1, wherein the admixture is free of a redox mediator and/or is free of an added redox mediator.

10. The method of claim 1, wherein the admixture comprising the waste source comprises an amount of the waste source such that 1 equiv to 10 equiv of the halogen containing polymer repeat unit is present in the admixture relative to an amount of the organic compound.

11. The method of claim 1, wherein the voltage is about −1V vs Ag/Ag⁺ to about −3 V vs Ag/Ag⁺.

12. A method for halogenation of an organic compound with a reactive halogen species from a waste source comprising at least one halogen containing polymer, comprising:
admixing the waste source with a solvent, and an electrolyte, wherein the solvent swells and/or dissolves the halogen containing polymer in the waste source;
exposing the admixture to a negative voltage in an electrochemical cell comprising a cathode by which the halogen containing polymer is dehalogenated and halogen anions are generated, and an oppositely disposed anode by which the halogen anions are transformed into the reactive halogen species;
recovering the reactive halogen species from the electrochemical cell; and
admixing the reactive halogen species with the organic compound, wherein the reactive halogen species reacts with the organic compound to halogenate the organic compound.

13. The method of claim 12, wherein the admixing of the reactive halogen species with the organic compound is performed in a container physically separated from the electrochemical cell.

14. The method of claim 12, wherein the admixture further comprises a redox mediator.

15. The method of claim 12, wherein the admixture is free of a redox mediator and/or is free of an added redox mediator.

16. The method of claim 12, wherein recovering the reactive halogen species comprises removing the reactive halogen species from the electrochemical cell.

17. The method of claim 12, wherein the at least one halogen containing polymer comprises F, Cl, Br, and/or I.

18. A method for halogenation of an organic compound with a reactive halogen species from a waste source comprising at least one halogen containing polymer, comprising:
admixing the waste source with a solvent and an electrolyte, wherein the solvent swells and/or dissolves the halogen containing polymer in the waste source;
introducing the admixture comprising the waste source into a divided electrochemical cells comprising an anode and an oppositely disposed cathode separated from the anode by an anion exchange membrane, wherein the admixture comprising the waste source is introduced into a cathode-side of the divided electrochemical cell;
introducing an organic compound to be halogenated on an anode-side of the divided electrochemical cell;
applying a negative voltage to the admixture comprising the waste source in the electrochemical cell, whereby the halogen containing polymer is dehalogenated and halogen anions are generated and flow through the anion exchange membrane to the anode wherein the halogen anions are converted into the reactive halogen species,
wherein the reactive halogen species reacts with the organic compound present in the anode-side of the divided electrochemical cell to halogenate the organic compound.

19. The method of claim 18, wherein the admixture is free of a redox mediator and/or is free of an added redox mediator.

20. The method of claim 19, wherein the at least one halogen containing polymer comprises F, Cl, Br, and/or I.
